SEQUENCE LISTING (554 sequences)

SEQ ID NO: 1 - DNA, 50 bp, synthetic construct
  source 1..50: mol_type = other DNA
aagtgactgtcggatgtgataaggggtgtgaggatggaggacatcatagt

SEQ ID NO: 2 - DNA, 50 bp, synthetic construct
  source 1..50: mol_type = other DNA
aagtgactgtcattccgcgtagcactaatgttctcaggggacatcatagt

SEQ ID NO: 3 - DNA, 50 bp, synthetic construct
  source 1..50: mol_type = other DNA
aagtgactgttgccacacacactgtcagattggacacagaacatcatagt

SEQ ID NO: 4 - DNA, 50 bp, synthetic construct
  source 1..50: mol_type = other DNA
aagtgactgtacgactgctcctagcagtacataccaccagacatcatagt

SEQ ID NO: 5 - DNA, 50 bp, synthetic construct
  source 1..50: mol_type = other DNA
aagtgactgtaaggtagcttcagtggtccgacgactgtagacatcatagt

SEQ ID NO: 6 - DNA, 50 bp, synthetic construct
  source 1..50: mol_type = other DNA
aagtgactgtcagatggcaatcttcatcgtcttcacagccacatcatagt

SEQ ID NO: 7 - DNA, 50 bp, synthetic construct
  source 1..50: mol_type = other DNA
aagtgactgttggaaacagacaccgatgagcctcatgacgacatcatagt

SEQ ID NO: 8 - DNA, 50 bp, synthetic construct
  source 1..50: mol_type = other DNA
aagtgactgtgtctccatgtttcatgaaaggtaagatgacacatcatagt

SEQ ID NO: 9 - DNA, 50 bp, synthetic construct
  source 1..50: mol_type = other DNA
aagtgactgtgtgtagacacggtcagctagactctcaatgacatcatagt

SEQ ID NO: 10 - DNA, 50 bp, synthetic construct
  source 1..50: mol_type = other DNA
aagtgactgtgacatcaaggcatacagtccatccagacagacatcatagt

SEQ ID NO: 11 - DNA, 50 bp, synthetic construct
  source 1..50: mol_type = other DNA
aagtgactgttgaggcagctacaggaatccttagggtctgacatcatagt

SEQ ID NO: 12 - DNA, 50 bp, synthetic construct
  source 1..50: mol_type = other DNA
aagtgactgtcctttgaatctagactctttaaaccttggaacatcatagt

SEQ ID NO: 13 - DNA, 50 bp, synthetic construct
  source 1..50: mol_type = other DNA
aagtgactgtaccaaagactcacagcatctttgaaacctaacatcatagt

SEQ ID NO: 14 - DNA, 50 bp, synthetic construct
  source 1..50: mol_type = other DNA
aagtgactgtgaattagagactttggaatttcaggtccttacatcatagt

SEQ ID NO: 15 - DNA, 50 bp, synthetic construct
  source 1..50: mol_type = other DNA
aagtgactgtaatgtcagagctttagaacagtaggtcttaacatcatagt

SEQ ID NO: 16 - DNA, 50 bp, synthetic construct
  source 1..50: mol_type = other DNA
aagtgactgtgaactttagaacctaaaaccttgaggctttacatcatagt

SEQ ID NO: 17 - DNA, 50 bp, synthetic construct
  source 1..50: mol_type = other DNA
aagtgactgtatctagactcatagagccttagctcctaaaacatcatagt

SEQ ID NO: 18 - DNA, 50 bp, synthetic construct
  source 1..50: mol_type = other DNA
aagtgactgttcttacattttaaggactctgaactctagaacatcatagt

SEQ ID NO: 19 - DNA, 50 bp, synthetic construct
  source 1..50: mol_type = other DNA
aagtgactgtcctccatgtctagaactatagaatctttagacatcatagt

SEQ ID NO: 20 - DNA, 50 bp, synthetic construct
  source 1..50: mol_type = other DNA
aagtgactgtaacatcacattttagaatcctaggccttagacatcatagt

SEQ ID NO: 21 - DNA, 50 bp, synthetic construct
  source 1..50: mol_type = other DNA
aagtgactgtaacattttgaagccttatgatctagagcctacatcatagt

SEQ ID NO: 22 - DNA, 50 bp, synthetic construct
  source 1..50: mol_type = other DNA
aagtgactgtgcctcagaaactataattgatcatcattagacatcatagt

SEQ ID NO: 23 - DNA, 50 bp, synthetic construct
  source 1..50: mol_type = other DNA
aagtgactgtttcgacccttaggatctaggatcttatacaacatcatagt

SEQ ID NO: 24 - DNA, 50 bp, synthetic construct
  source 1..50: mol_type = other DNA
aagtgactgtagaatatcacatcttagaacaccggaccttacatcatagt

SEQ ID NO: 25 - DNA, 50 bp, synthetic construct
  source 1..50: mol_type = other DNA
aagtgactgtcaatggaatctagagtgccttgaaacagaaacatcatagt

SEQ ID NO: 26 - DNA, 50 bp, synthetic construct
  source 1..50: mol_type = other DNA
aagtgactgtaacttagatgcttaggatccggaatcttggacatcatagt

SEQ ID NO: 27 - DNA, 50 bp, synthetic construct
  source 1..50: mol_type = other DNA
aagtgactgtaatgtggattcttggactcttaaccatggtacatcatagt

SEQ ID NO: 28 - DNA, 50 bp, synthetic construct
  source 1..50: mol_type = other DNA
aagtgactgtcatttgagtcttagaactacagtgcctagaacatcatagt

SEQ ID NO: 29 - DNA, 50 bp, synthetic construct
  source 1..50: mol_type = other DNA
aagtgactgtgtctagacctgtggacctttagaatcttagacatcatagt

SEQ ID NO: 30 - DNA, 50 bp, synthetic construct
  source 1..50: mol_type = other DNA
aagtgactgtgtatagggttagaaccttgaaattgcacctacatcatagt

SEQ ID NO: 31 - DNA, 50 bp, synthetic construct
  source 1..50: mol_type = other DNA
aagtgactgttgttactcgtgccttggcaaactttcttttacatcatagt

SEQ ID NO: 32 - DNA, 50 bp, synthetic construct
  source 1..50: mol_type = other DNA
aagtgactgtatgatcttcaaaagtgcccaactgcgtgagacatcatagt

SEQ ID NO: 33 - DNA, 50 bp, synthetic construct
  source 1..50: mol_type = other DNA
aagtgactgtcataggtaatttccaaattcccaaggaccaacatcatagt

SEQ ID NO: 34 - DNA, 50 bp, synthetic construct
  source 1..50: mol_type = other DNA
aagtgactgtcaggttttccaaaggaattcgctccactgtacatcatagt

SEQ ID NO: 35 - DNA, 50 bp, synthetic construct
  source 1..50: mol_type = other DNA
aagtgactgtggaattttcgtagtacatatttcctctgatacatcatagt

SEQ ID NO: 36 - DNA, 50 bp, synthetic construct
  source 1..50: mol_type = other DNA
aagtgactgtgcatcatagttagataagactgctaaggcaacatcatagt

SEQ ID NO: 37 - DNA, 50 bp, synthetic construct
  source 1..50: mol_type = other DNA
aagtgactgttccttgcacgtggcttcgtctcggaatttgacatcatagt

SEQ ID NO: 38 - DNA, 50 bp, synthetic construct
  source 1..50: mol_type = other DNA
aagtgactgtctggtacgtggtggggttgtagagcatgagacatcatagt

SEQ ID NO: 39 - DNA, 50 bp, synthetic construct
  source 1..50: mol_type = other DNA
aagtgactgtacataattacggggacacttcttcacgcagacatcatagt

SEQ ID NO: 40 - DNA, 50 bp, synthetic construct
  source 1..50: mol_type = other DNA
aagtgactgtattcaccaatacctattccgttacacacttacatcatagt

SEQ ID NO: 41 - DNA, 50 bp, synthetic construct
  source 1..50: mol_type = other DNA
aagtgactgtattcgtagcatttatggagagtgagtctttacatcatagt

SEQ ID NO: 42 - DNA, 50 bp, synthetic construct
  source 1..50: mol_type = other DNA
aagtgactgtcactgatggaggtgcagtttttgaagtgttacatcatagt

SEQ ID NO: 43 - DNA, 50 bp, synthetic construct
  source 1..50: mol_type = other DNA
aagtgactgtatgtgtgaaggagtcacccctaaatgccacacatcatagt

SEQ ID NO: 44 - DNA, 50 bp, synthetic construct
  source 1..50: mol_type = other DNA
aagtgactgtaaaaccctgtgatttcctttacggttttcaacatcatagt

SEQ ID NO: 45 - DNA, 50 bp, synthetic construct
  source 1..50: mol_type = other DNA
aagtgactgtcgcgtatgatttctaggttctcaaaggcatacatcatagt

SEQ ID NO: 46 - DNA, 50 bp, synthetic construct
  source 1..50: mol_type = other DNA
aagtgactgtaagagaaaactgaccatgttgcttggtcctacatcatagt

SEQ ID NO: 47 - DNA, 50 bp, synthetic construct
  source 1..50: mol_type = other DNA
aagtgactgttatcacatctccatcacttatctccttgagacatcatagt

SEQ ID NO: 48 - DNA, 50 bp, synthetic construct
  source 1..50: mol_type = other DNA
aagtgactgtcactgtatgcactcagagttctccacaaacacatcatagt

SEQ ID NO: 49 - DNA, 50 bp, synthetic construct
  source 1..50: mol_type = other DNA
aagtgactgtcgaacgatgtggcgccttcgcatgaagaggacatcatagt

SEQ ID NO: 50 - DNA, 50 bp, synthetic construct
  source 1..50: mol_type = other DNA
aagtgactgttgaattcagtttccttcaagatcctcaagaacatcatagt

SEQ ID NO: 51 - DNA, 50 bp, synthetic construct
  source 1..50: mol_type = other DNA
aagtgactgttccttgatagcgacgggaattttaactttcacatcatagt

SEQ ID NO: 52 - DNA, 50 bp, synthetic construct
  source 1..50: mol_type = other DNA
aagtgactgttgttggctttcggagatgttgcttctcttaacatcatagt

SEQ ID NO: 53 - DNA, 50 bp, synthetic construct
  source 1..50: mol_type = other DNA
aagtgactgttggccatcacgtaggcttcatcgaggatttacatcatagt

SEQ ID NO: 54 - DNA, 50 bp, synthetic construct
  source 1..50: mol_type = other DNA
aagtgactgtaatattgtctttgtgttcccggacatagtcacatcatagt

SEQ ID NO: 55 - DNA, 50 bp, synthetic construct
  source 1..50: mol_type = other DNA
aagtgactgtcttctgcatggtattctttctcttccgcacacatcatagt

SEQ ID NO: 56 - DNA, 50 bp, synthetic construct
  source 1..50: mol_type = other DNA
aagtgactgtaattgattccaatgccatccacttgataggacatcatagt

SEQ ID NO: 57 - DNA, 50 bp, synthetic construct
  source 1..50: mol_type = other DNA
aagtgactgtaactttgggcgactatctgcgtctatcatcacatcatagt

SEQ ID NO: 58 - DNA, 50 bp, synthetic construct
  source 1..50: mol_type = other DNA
aagtgactgttcgggccattttggagaattcgatgatcaaacatcatagt

SEQ ID NO: 59 - DNA, 50 bp, synthetic construct
  source 1..50: mol_type = other DNA
aagtgactgtaatgcattctttcatccccctgaatgacaaacatcatagt

SEQ ID NO: 60 - DNA, 50 bp, synthetic construct
  source 1..50: mol_type = other DNA
aagtgactgtagctttgcagcccatttctatcaatgcaagacatcatagt

SEQ ID NO: 61 - DNA, 50 bp, synthetic construct
  source 1..50: mol_type = other DNA
aagtgactgtactaccaggaatcgggtatccaggttgaagacatcatagt

SEQ ID NO: 62 - DNA, 50 bp, synthetic construct
  source 1..50: mol_type = other DNA
aagtgactgtcagggtcatttttcactgtgatgttcatccacatcatagt

SEQ ID NO: 63 - DNA, 50 bp, synthetic construct
  source 1..50: mol_type = other DNA
aagtgactgttccaagccacatgtcctcaataatgtgatgacatcatagt

SEQ ID NO: 64 - DNA, 50 bp, synthetic construct
  source 1..50: mol_type = other DNA
aagtgactgttctaggtcattgcctcgcacgtagcacttgacatcatagt

SEQ ID NO: 65 - DNA, 50 bp, synthetic construct
  source 1..50: mol_type = other DNA
aagtgactgtttgtggtaggtctgccagtcatcactggagacatcatagt

SEQ ID NO: 66 - DNA, 50 bp, synthetic construct
  source 1..50: mol_type = other DNA
aagtgactgttccaggtagtctgtgttgctattgcacatcacatcatagt

SEQ ID NO: 67 - DNA, 50 bp, synthetic construct
  source 1..50: mol_type = other DNA
aagtgactgttgaatcatgtagctgtttcctttccagttgacatcatagt

SEQ ID NO: 68 - DNA, 50 bp, synthetic construct
  source 1..50: mol_type = other DNA
aagtgactgttgtaactatactcagataagtcccactcctacatcatagt

SEQ ID NO: 69 - DNA, 50 bp, synthetic construct
  source 1..50: mol_type = other DNA
aagtgactgtaatatagaggtttccttggtcctctgggtcacatcatagt

SEQ ID NO: 70 - DNA, 50 bp, synthetic construct
  source 1..50: mol_type = other DNA
aagtgactgtaggatgaagctgcctacctgcatcgtgtacacatcatagt

SEQ ID NO: 71 - DNA, 50 bp, synthetic construct
  source 1..50: mol_type = other DNA
aagtgactgtcacctgtcacaatggtgatgtttttggggtacatcatagt

SEQ ID NO: 72 - DNA, 50 bp, synthetic construct
  source 1..50: mol_type = other DNA
aagtgactgtatgctggccacagataaaccaaaggcagagacatcatagt

SEQ ID NO: 73 - DNA, 50 bp, synthetic construct
  source 1..50: mol_type = other DNA
aagtgactgtagagctactgtgatagatgtacactttgccacatcatagt

SEQ ID NO: 74 - DNA, 50 bp, synthetic construct
  source 1..50: mol_type = other DNA
aagtgactgtcaccaaggtcttgtggacgatgttgatgacacatcatagt

SEQ ID NO: 75 - DNA, 50 bp, synthetic construct
  source 1..50: mol_type = other DNA
aagtgactgtcagctcgcaaaagatggtctcattagcttgacatcatagt

SEQ ID NO: 76 - DNA, 50 bp, synthetic construct
  source 1..50: mol_type = other DNA
aagtgactgtaaagaagctttgtagccggtgatttaccatacatcatagt

SEQ ID NO: 77 - DNA, 50 bp, synthetic construct
  source 1..50: mol_type = other DNA
aagtgactgttacatcctccacagttttcatgccagactcacatcatagt

SEQ ID NO: 78 - DNA, 50 bp, synthetic construct
  source 1..50: mol_type = other DNA
aagtgactgtatggacggtgatctccgtgggatacagcagacatcatagt

SEQ ID NO: 79 - DNA, 50 bp, synthetic construct
  source 1..50: mol_type = other DNA
aagtgactgtggtcagaaagagtgaggttgagagggttgaacatcatagt

SEQ ID NO: 80 - DNA, 50 bp, synthetic construct
  source 1..50: mol_type = other DNA
aagtgactgtgtcagcacagtctcagacttggcttttttgacatcatagt

SEQ ID NO: 81 - DNA, 50 bp, synthetic construct
  source 1..50: mol_type = other DNA
aagtgactgtcagtcacgttggtgacaacgggggcatcagacatcatagt

SEQ ID NO: 82 - DNA, 50 bp, synthetic construct
  source 1..50: mol_type = other DNA
aagtgactgttttacccggactcggtcaaagtctctgtaaacatcatagt

SEQ ID NO: 83 - DNA, 50 bp, synthetic construct
  source 1..50: mol_type = other DNA
aagtgactgttcaatgtccacagagaaccacgtggtcttgacatcatagt

SEQ ID NO: 84 - DNA, 50 bp, synthetic construct
  source 1..50: mol_type = other DNA
aagtgactgtgtggtacttgggcatgatctgataatagcgacatcatagt

SEQ ID NO: 85 - DNA, 50 bp, synthetic construct
  source 1..50: mol_type = other DNA
aagtgactgtaggacgtcagtagtattggtcccgagtctgacatcatagt

SEQ ID NO: 86 - DNA, 50 bp, synthetic construct
  source 1..50: mol_type = other DNA
aagtgactgtcatgtgcatgctcttgacgaggtgtatgtgacatcatagt

SEQ ID NO: 87 - DNA, 50 bp, synthetic construct
  source 1..50: mol_type = other DNA
aagtgactgtacaggaatgcagtgcactcggcgttgtaagacatcatagt

SEQ ID NO: 88 - DNA, 50 bp, synthetic construct
  source 1..50: mol_type = other DNA
aagtgactgtttgacagcaaagtcaatccagtgactgtggacatcatagt

SEQ ID NO: 89 - DNA, 50 bp, synthetic construct
  source 1..50: mol_type = other DNA
aagtgactgtttctttctagttcctttgctgttgggtctgacatcatagt

SEQ ID NO: 90 - DNA, 50 bp, synthetic construct
  source 1..50: mol_type = other DNA
aagtgactgttatcctcccaacatctgtctgtccagaattacatcatagt

SEQ ID NO: 91 - DNA, 50 bp, synthetic construct
  source 1..50: mol_type = other DNA
aagtgactgtctggtagtggtgagtgagaagtggcatctcacatcatagt

SEQ ID NO: 92 - DNA, 50 bp, synthetic construct
  source 1..50: mol_type = other DNA
aagtgactgtctattctacattcaagtgtcgctccctccgacatcatagt

SEQ ID NO: 93 - DNA, 50 bp, synthetic construct
  source 1..50: mol_type = other DNA
aagtgactgtgaagaacttggaagacattgcacttccagcacatcatagt

SEQ ID NO: 94 - DNA, 50 bp, synthetic construct
  source 1..50: mol_type = other DNA
aagtgactgtgaccaccaagaattggcttcaattacaaccacatcatagt

SEQ ID NO: 95 - DNA, 50 bp, synthetic construct
  source 1..50: mol_type = other DNA
aagtgactgttctgacatctgaacagggttattcatacctacatcatagt

SEQ ID NO: 96 - DNA, 50 bp, synthetic construct
  source 1..50: mol_type = other DNA
aagtgactgtgatgtactgcatgtggtcctgatacaagtgacatcatagt

SEQ ID NO: 97 - DNA, 50 bp, synthetic construct
  source 1..50: mol_type = other DNA
aagtgactgttgctgcagttccaccttcgatgtcggaattacatcatagt

SEQ ID NO: 98 - DNA, 50 bp, synthetic construct
  source 1..50: mol_type = other DNA
aagtgactgtcatcaccctgccaaaaacagaggtgttatcacatcatagt

SEQ ID NO: 99 - DNA, 50 bp, synthetic construct
  source 1..50: mol_type = other DNA
aagtgactgtttggcaaagcggtagccatagtcgatgttgacatcatagt

SEQ ID NO: 100 - DNA, 50 bp, synthetic construct
  source 1..50: mol_type = other DNA
aagtgactgtttcatgaggatgcgagcactctcgtaggagacatcatagt

SEQ ID NO: 101 - DNA, 50 bp, synthetic construct
  source 1..50: mol_type = other DNA
aagtgactgtatgtgtaccatgtccacacagctaagttagacatcatagt

SEQ ID NO: 102 - DNA, 50 bp, synthetic construct
  source 1..50: mol_type = other DNA
aagtgactgtctcaaataggttgtctgctctggtgcagtgacatcatagt

SEQ ID NO: 103 - DNA, 50 bp, synthetic construct
  source 1..50: mol_type = other DNA
aagtgactgtatgtttcatttcctgtcctacaaccaaaggacatcatagt

SEQ ID NO: 104 - DNA, 50 bp, synthetic construct
  source 1..50: mol_type = other DNA
aagtgactgttgtgaattccattagccaagttctgtcattacatcatagt

SEQ ID NO: 105 - DNA, 50 bp, synthetic construct
  source 1..50: mol_type = other DNA
aagtgactgttgggaacagttttaaggtacacctgcagtaacatcatagt

SEQ ID NO: 106 - DNA, 50 bp, synthetic construct
  source 1..50: mol_type = other DNA
aagtgactgtatgctctagacagagaaataaccccagagtacatcatagt

SEQ ID NO: 107 - DNA, 50 bp, synthetic construct
  source 1..50: mol_type = other DNA
aagtgactgttgtctataacgaacaagtaatgccctctccacatcatagt

SEQ ID NO: 108 - DNA, 50 bp, synthetic construct
  source 1..50: mol_type = other DNA
aagtgactgtcatttctaataggcatgggtttccattctgacatcatagt

SEQ ID NO: 109 - DNA, 50 bp, synthetic construct
  source 1..50: mol_type = other DNA
aagtgactgttagtgggctgaatattccttagggactcaaacatcatagt

SEQ ID NO: 110 - DNA, 50 bp, synthetic construct
  source 1..50: mol_type = other DNA
aagtgactgtcacagttggctgaggtgatatgaaagccaaacatcatagt

SEQ ID NO: 111 - DNA, 50 bp, synthetic construct
  source 1..50: mol_type = other DNA
aagtgactgtagtgctttttgctttcaagatcttttgctcacatcatagt

SEQ ID NO: 112 - DNA, 50 bp, synthetic construct
  source 1..50: mol_type = other DNA
aagtgactgtcgtcatggtttctccaaaaatctccacatgacatcatagt

SEQ ID NO: 113 - DNA, 50 bp, synthetic construct
  source 1..50: mol_type = other DNA
aagtgactgttcttttattactttgacatgtagtcatcctacatcatagt

SEQ ID NO: 114 - DNA, 50 bp, synthetic construct
  source 1..50: mol_type = other DNA
aagtgactgtaaaccttttgaagcttccaaatagcttggtacatcatagt

SEQ ID NO: 115 - DNA, 50 bp, synthetic construct
  source 1..50: mol_type = other DNA
aagtgactgtagagaattccccttttgttccattacatatacatcatagt

SEQ ID NO: 116 - DNA, 50 bp, synthetic construct
  source 1..50: mol_type = other DNA
aagtgactgtaaatctggtcacggataaaagcatgtgcctacatcatagt

SEQ ID NO: 117 - DNA, 50 bp, synthetic construct
  source 1..50: mol_type = other DNA
aagtgactgttcaaatagagaggtgcaatagttgcagtggacatcatagt

SEQ ID NO: 118 - DNA, 50 bp, synthetic construct
  source 1..50: mol_type = other DNA
aagtgactgtaacctttagtttccaaccagttttcattctacatcatagt

SEQ ID NO: 119 - DNA, 50 bp, synthetic construct
  source 1..50: mol_type = other DNA
aagtgactgtactgtccagatttccgtgtacatttttgaaacatcatagt

SEQ ID NO: 120 - DNA, 50 bp, synthetic construct
  source 1..50: mol_type = other DNA
aagtgactgtatgcattcttggcatccttaaaaggtttcaacatcatagt

SEQ ID NO: 121 - DNA, 50 bp, synthetic construct
  source 1..50: mol_type = other DNA
aagtgactgtctatatgttggtctgctgtttttttgtggaacatcatagt

SEQ ID NO: 122 - DNA, 50 bp, synthetic construct
  source 1..50: mol_type = other DNA
aagtgactgtgtcctagaaccacaagagtttgaatttgccacatcatagt

SEQ ID NO: 123 - DNA, 50 bp, synthetic construct
  source 1..50: mol_type = other DNA
aagtgactgtgcgtcatatttgataattctagtggcttgaacatcatagt

SEQ ID NO: 124 - DNA, 50 bp, synthetic construct
  source 1..50: mol_type = other DNA
aagtgactgtaccggtctcttattcagagtatttactctgacatcatagt

SEQ ID NO: 125 - DNA, 50 bp, synthetic construct
  source 1..50: mol_type = other DNA
aagtgactgtctcccactgttgatttatctgatagtgtttacatcatagt

SEQ ID NO: 126 - DNA, 50 bp, synthetic construct
  source 1..50: mol_type = other DNA
aagtgactgtacaagtacaaaccaacgcattgcctaggtaacatcatagt

SEQ ID NO: 127 - DNA, 50 bp, synthetic construct
  source 1..50: mol_type = other DNA
aagtgactgttactctcgcagttaaaacctcggcttcctcacatcatagt

SEQ ID NO: 128 - DNA, 50 bp, synthetic construct
  source 1..50: mol_type = other DNA
aagtgactgttcataagtgtcacccactcggtaagtgttcacatcatagt

SEQ ID NO: 129 - DNA, 50 bp, synthetic construct
  source 1..50: mol_type = other DNA
aagtgactgttacagtcccagatcatggagtctttaggacacatcatagt

SEQ ID NO: 130 - DNA, 50 bp, synthetic construct
  source 1..50: mol_type = other DNA
aagtgactgtcagcggtttgcgatggtacagcttattctcacatcatagt

SEQ ID NO: 131 - DNA, 50 bp, synthetic construct
  source 1..50: mol_type = other DNA
aagtgactgttccaggtgtcaccaatcttgtaggactgacacatcatagt

SEQ ID NO: 132 - DNA, 50 bp, synthetic construct
  source 1..50: mol_type = other DNA
aagtgactgttaacatgtaaccaccagtctcatgtggtctacatcatagt

SEQ ID NO: 133 - DNA, 50 bp, synthetic construct
  source 1..50: mol_type = other DNA
aagtgactgttctcccacgtttctccgaccacataggaagacatcatagt

SEQ ID NO: 134 - DNA, 50 bp, synthetic construct
  source 1..50: mol_type = other DNA
aagtgactgtcaagtacaatctaccatcatccagccttggacatcatagt

SEQ ID NO: 135 - DNA, 50 bp, synthetic construct
  source 1..50: mol_type = other DNA
aagtgactgtctatttctagaagtgcaagtgatgcgtccgacatcatagt

SEQ ID NO: 136 - DNA, 50 bp, synthetic construct
  source 1..50: mol_type = other DNA
aagtgactgtagtagaccacaccactgtctgtgacacagtacatcatagt

SEQ ID NO: 137 - DNA, 50 bp, synthetic construct
  source 1..50: mol_type = other DNA
aagtgactgttaagacacatggctctccatttgagttgccacatcatagt

SEQ ID NO: 138 - DNA, 50 bp, synthetic construct
  source 1..50: mol_type = other DNA
aagtgactgtgagtagaacgtcctgccattgtaggtgaatacatcatagt

SEQ ID NO: 139 - DNA, 50 bp, synthetic construct
  source 1..50: mol_type = other DNA
aagtgactgtaattcgaagttgtgctgcaccaaagatgtcacatcatagt

SEQ ID NO: 140 - DNA, 50 bp, synthetic construct
  source 1..50: mol_type = other DNA
aagtgactgttgtgcagaaagagtatttctggtcctgctcacatcatagt

SEQ ID NO: 141 - DNA, 50 bp, synthetic construct
  source 1..50: mol_type = other DNA
aagtgactgtaaggcaccattggaatttcctcctcgagtcacatcatagt

SEQ ID NO: 142 - DNA, 50 bp, synthetic construct
  source 1..50: mol_type = other DNA
aagtgactgttcttctgccctcagaagtgcaatcagtgtaacatcatagt

SEQ ID NO: 143 - DNA, 50 bp, synthetic construct
  source 1..50: mol_type = other DNA
aagtgactgtttctgtgtggtcccacaccacttcatgttgacatcatagt

SEQ ID NO: 144 - DNA, 50 bp, synthetic construct
  source 1..50: mol_type = other DNA
aagtgactgtggcagaacccaaacttctggtcggcatcatacatcatagt

SEQ ID NO: 145 - DNA, 50 bp, synthetic construct
  source 1..50: mol_type = other DNA
aagtgactgttacatgaccccttcattggttgtgcagattacatcatagt

SEQ ID NO: 146 - DNA, 50 bp, synthetic construct
  source 1..50: mol_type = other DNA
aagtgactgttcatgctgcttatcccactgatctccaatgacatcatagt

SEQ ID NO: 147 - DNA, 50 bp, synthetic construct
  source 1..50: mol_type = other DNA
aagtgactgtaagctgcgagtaggcaatgcatgtccattcacatcatagt

SEQ ID NO: 148 - DNA, 50 bp, synthetic construct
  source 1..50: mol_type = other DNA
aagtgactgtgtaagtgatgtcatcaacaatgcactgatcacatcatagt

SEQ ID NO: 149 - DNA, 50 bp, synthetic construct
  source 1..50: mol_type = other DNA
aagtgactgtttcatgacgcttgtggaatgtgtcgttcacacatcatagt

SEQ ID NO: 150 - DNA, 50 bp, synthetic construct
  source 1..50: mol_type = other DNA
aagtgactgtctgaccgaagcatgtacagttcagcatgtgacatcatagt

SEQ ID NO: 151 - DNA, 50 bp, synthetic construct
  source 1..50: mol_type = other DNA
aagtgactgtaaacgtcccagtctctgaatcctggcattgacatcatagt

SEQ ID NO: 152 - DNA, 50 bp, synthetic construct
  source 1..50: mol_type = other DNA
aagtgactgtacatacttctcccatgaatctccaatttgaacatcatagt

SEQ ID NO: 153 - DNA, 50 bp, synthetic construct
  source 1..50: mol_type = other DNA
aagtgactgtcatagcagtagcactggtatctgacaccatacatcatagt

SEQ ID NO: 154 - DNA, 50 bp, synthetic construct
  source 1..50: mol_type = other DNA
aagtgactgtgactcggagtctcagtgataaatacttcgaacatcatagt

SEQ ID NO: 155 - DNA, 50 bp, synthetic construct
  source 1..50: mol_type = other DNA
aagtgactgtatactcacaggtctccacctgagaatgtacacatcatagt

SEQ ID NO: 156 - DNA, 50 bp, synthetic construct
  source 1..50: mol_type = other DNA
aagtgactgttaggaatagttaatcagagttgttggctcaacatcatagt

SEQ ID NO: 157 - DNA, 50 bp, synthetic construct
  source 1..50: mol_type = other DNA
aagtgactgtaaagtccagaataacctgcatggtctagggacatcatagt

SEQ ID NO: 158 - DNA, 50 bp, synthetic construct
  source 1..50: mol_type = other DNA
aagtgactgtcagacagatcagaaatgatatcggttgggaacatcatagt

SEQ ID NO: 159 - DNA, 50 bp, synthetic construct
  source 1..50: mol_type = other DNA
aagtgactgtgcaaatctgtagaatttcttcacattcctgacatcatagt

SEQ ID NO: 160 - DNA, 50 bp, synthetic construct
  source 1..50: mol_type = other DNA
aagtgactgtagcaagtttcaaagttttgggccagttttcacatcatagt

SEQ ID NO: 161 - DNA, 50 bp, synthetic construct
  source 1..50: mol_type = other DNA
aagtgactgttgtatgtcagaagtttccatcttatcctcaacatcatagt

SEQ ID NO: 162 - DNA, 50 bp, synthetic construct
  source 1..50: mol_type = other DNA
aagtgactgtcacttctgaaggtggacatgaattctcactacatcatagt

SEQ ID NO: 163 - DNA, 50 bp, synthetic construct
  source 1..50: mol_type = other DNA
aagtgactgtacagatttctgctgttcatacactgggatcacatcatagt

SEQ ID NO: 164 - DNA, 50 bp, synthetic construct
  source 1..50: mol_type = other DNA
aagtgactgtctgtaactctatacccatgtctttcaaagtacatcatagt

SEQ ID NO: 165 - DNA, 50 bp, synthetic construct
  source 1..50: mol_type = other DNA
aagtgactgtatgatgtcattgttctcaacaatctgttccacatcatagt

SEQ ID NO: 166 - DNA, 50 bp, synthetic construct
  source 1..50: mol_type = other DNA
aagtgactgtatcgttccctttttaaggttgttcacaagaacatcatagt

SEQ ID NO: 167 - DNA, 50 bp, synthetic construct
  source 1..50: mol_type = other DNA
aagtgactgttgttgtttactagtgttgtggcattcatcaacatcatagt

SEQ ID NO: 168 - DNA, 50 bp, synthetic construct
  source 1..50: mol_type = other DNA
aagtgactgttaatccaaggcttcatctgtgtttttggcaacatcatagt

SEQ ID NO: 169 - DNA, 50 bp, synthetic construct
  source 1..50: mol_type = other DNA
aagtgactgtccgtgattccactttcctgaaaaacttctgacatcatagt

SEQ ID NO: 170 - DNA, 50 bp, synthetic construct
  source 1..50: mol_type = other DNA
aagtgactgtgctgagctatgatgtatttaaatttgtcgcacatcatagt

SEQ ID NO: 171 - DNA, 50 bp, synthetic construct
  source 1..50: mol_type = other DNA
aagtgactgtaatttgagataagttttcgaggtctttgcaacatcatagt

SEQ ID NO: 172 - DNA, 50 bp, synthetic construct
  source 1..50: mol_type = other DNA
aagtgactgttgttcatatttctgtgttccaaattccctaacatcatagt

SEQ ID NO: 173 - DNA, 50 bp, synthetic construct
  source 1..50: mol_type = other DNA
aagtgactgtctttacaaatcctgctctcttcatctttgtacatcatagt

SEQ ID NO: 174 - DNA, 50 bp, synthetic construct
  source 1..50: mol_type = other DNA
aagtgactgtgggcatcattatatttccgcaaatgtgaagacatcatagt

SEQ ID NO: 175 - DNA, 50 bp, synthetic construct
  source 1..50: mol_type = other DNA
aagtgactgtcatctttcattcgtgcatgctcactgataaacatcatagt

SEQ ID NO: 176 - DNA, 50 bp, synthetic construct
  source 1..50: mol_type = other DNA
aagtgactgtaagatcttgctcaatctcatcgaatcctgcacatcatagt

SEQ ID NO: 177 - DNA, 50 bp, synthetic construct
  source 1..50: mol_type = other DNA
aagtgactgttttctagttcctgcagcttttcttcaaatcacatcatagt

SEQ ID NO: 178 - DNA, 50 bp, synthetic construct
  source 1..50: mol_type = other DNA
aagtgactgtgaggtcttcaagtttaggattctcattgctacatcatagt

SEQ ID NO: 179 - DNA, 50 bp, synthetic construct
  source 1..50: mol_type = other DNA
aagtgactgttccacaagtgctctggttttcacaaagagaacatcatagt

SEQ ID NO: 180 - DNA, 50 bp, synthetic construct
  source 1..50: mol_type = other DNA
aagtgactgtacgtccagtcaatatgccaggttttagaaaacatcatagt

SEQ ID NO: 181 - DNA, 50 bp, synthetic construct
  source 1..50: mol_type = other DNA
aagtgactgttcattttgatgacattgcccacatactcatacatcatagt

SEQ ID NO: 182 - DNA, 50 bp, synthetic construct
  source 1..50: mol_type = other DNA
aagtgactgtttcgtcccatgtctgaaggcgtaaaatagaacatcatagt

SEQ ID NO: 183 - DNA, 50 bp, synthetic construct
  source 1..50: mol_type = other DNA
aagtgactgtatgagtctgtatatgcagaatcttttccctacatcatagt

SEQ ID NO: 184 - DNA, 50 bp, synthetic construct
  source 1..50: mol_type = other DNA
aagtgactgtggtacaggttttggtttttcttgactatctacatcatagt

SEQ ID NO: 185 - DNA, 50 bp, synthetic construct
  source 1..50: mol_type = other DNA
aagtgactgttaatggcattcctctatcactcttacgtcaacatcatagt

SEQ ID NO: 186 - DNA, 50 bp, synthetic construct
  source 1..50: mol_type = other DNA
aagtgactgttcgggcacagaatatctttgctcttttttcacatcatagt

SEQ ID NO: 187 - DNA, 50 bp, synthetic construct
  source 1..50: mol_type = other DNA
aagtgactgttcaaatgtcttgtacttcacatggattcccacatcatagt

SEQ ID NO: 188 - DNA, 50 bp, synthetic construct
  source 1..50: mol_type = other DNA
aagtgactgtgagtacagtgtctgaactccagttgcaataacatcatagt

SEQ ID NO: 189 - DNA, 50 bp, synthetic construct
  source 1..50: mol_type = other DNA
aagtgactgttcttcagcggttgacttttgagttcgtaatacatcatagt

SEQ ID NO: 190 - DNA, 50 bp, synthetic construct
  source 1..50: mol_type = other DNA
aagtgactgtatactggaggagcttatcggaggcttggagacatcatagt

SEQ ID NO: 191 - DNA, 50 bp, synthetic construct
  source 1..50: mol_type = other DNA
aagtgactgtatgagttgctggcgtagcaagatgcgtgatacatcatagt

SEQ ID NO: 192 - DNA, 50 bp, synthetic construct
  source 1..50: mol_type = other DNA
aagtgactgtactgacgtttatggctggtgtctgactcacacatcatagt

SEQ ID NO: 193 - DNA, 50 bp, synthetic construct
  source 1..50: mol_type = other DNA
aagtgactgtcgttgggcttgctgtatgtggtacttggtgacatcatagt

SEQ ID NO: 194 - DNA, 50 bp, synthetic construct
  source 1..50: mol_type = other DNA
aagtgactgtaagtggtagaaaggtactgctttacctgctacatcatagt

SEQ ID NO: 195 - DNA, 50 bp, synthetic construct
  source 1..50: mol_type = other DNA
aagtgactgtaagctcaggacttggttggcatgtttatttacatcatagt

SEQ ID NO: 196 - DNA, 50 bp, synthetic construct
  source 1..50: mol_type = other DNA
aagtgactgtatacaggacgctcgtgaatgtgtgttcatgacatcatagt

SEQ ID NO: 197 - DNA, 50 bp, synthetic construct
  source 1..50: mol_type = other DNA
aagtgactgtggctaatgatgtcatcgattacatcatccaacatcatagt

SEQ ID NO: 198 - DNA, 50 bp, synthetic construct
  source 1..50: mol_type = other DNA
aagtgactgtccatcaagcccaagatttcctcattataacacatcatagt

SEQ ID NO: 199 - DNA, 50 bp, synthetic construct
  source 1..50: mol_type = other DNA
aagtgactgtgagacaggcaacgtatttgccatttgcaaaacatcatagt

SEQ ID NO: 200 - DNA, 50 bp, synthetic construct
  source 1..50: mol_type = other DNA
aagtgactgtggcagaccttggtttccataaagatcaatcacatcatagt

SEQ ID NO: 201 - DNA, 50 bp, synthetic construct
  source 1..50: mol_type = other DNA
aagtgactgttcagactctgtgagctccctttttatgttgacatcatagt

SEQ ID NO: 202 - DNA, 50 bp, synthetic construct
  source 1..50: mol_type = other DNA
aagtgactgttcttcttcgttcaatcaggttgtgattgtcacatcatagt

SEQ ID NO: 203 - DNA, 50 bp, synthetic construct
  source 1..50: mol_type = other DNA
aagtgactgtacctagttctttaatgcggtcatttatgttacatcatagt

SEQ ID NO: 204 - DNA, 50 bp, synthetic construct
  source 1..50: mol_type = other DNA
aagtgactgtgcatgtctggatcatttgacttgggaatcaacatcatagt

SEQ ID NO: 205 - DNA, 50 bp, synthetic construct
  source 1..50: mol_type = other DNA
aagtgactgtacggatgcttttaagatggttcccttgttcacatcatagt

SEQ ID NO: 206 - DNA, 50 bp, synthetic construct
  source 1..50: mol_type = other DNA
aagtgactgttgctgttctcgttgcaactttcggatatagacatcatagt

SEQ ID NO: 207 - DNA, 50 bp, synthetic construct
  source 1..50: mol_type = other DNA
aagtgactgtcagtttcttctgtcggttttcaagttctttacatcatagt

SEQ ID NO: 208 - DNA, 50 bp, synthetic construct
  source 1..50: mol_type = other DNA
aagtgactgtatgagctcgagcctgcatttcaagttcctgacatcatagt

SEQ ID NO: 209 - DNA, 50 bp, synthetic construct
  source 1..50: mol_type = other DNA
aagtgactgtgcttgatgatccgattcaccaaatctggagacatcatagt

SEQ ID NO: 210 - DNA, 50 bp, synthetic construct
  source 1..50: mol_type = other DNA
aagtgactgtagtggatcagtgacaccgacgggagaaaggacatcatagt

SEQ ID NO: 211 - DNA, 50 bp, synthetic construct
  source 1..50: mol_type = other DNA
aagtgactgtattagactttatatttcagagccttggtctacatcatagt

SEQ ID NO: 212 - DNA, 50 bp, synthetic construct
  source 1..50: mol_type = other DNA
aagtgactgtccaattagaaatatgcccagttcggttttcacatcatagt

SEQ ID NO: 213 - DNA, 50 bp, synthetic construct
  source 1..50: mol_type = other DNA
aagtgactgtgggtgtacaaagaatgcggaaaaccaccagacatcatagt

SEQ ID NO: 214 - DNA, 50 bp, synthetic construct
  source 1..50: mol_type = other DNA
aagtgactgttcgcaaagctgtcttttaaatgcaagacctacatcatagt

SEQ ID NO: 215 - DNA, 50 bp, synthetic construct
  source 1..50: mol_type = other DNA
aagtgactgtgtgtccaaaaacagttctgagtgagactgtacatcatagt

SEQ ID NO: 216 - DNA, 50 bp, synthetic construct
  source 1..50: mol_type = other DNA
aagtgactgtatctccaagaatattgctattttcatgtccacatcatagt

SEQ ID NO: 217 - DNA, 50 bp, synthetic construct
  source 1..50: mol_type = other DNA
aagtgactgtccccaatttaaatgaccataatgggcgtatacatcatagt

SEQ ID NO: 218 - DNA, 50 bp, synthetic construct
  source 1..50: mol_type = other DNA
aagtgactgtaacattgttatcaagtaacagtaacttcccacatcatagt

SEQ ID NO: 219 - DNA, 50 bp, synthetic construct
  source 1..50: mol_type = other DNA
aagtgactgtgcactgaaactaactttataacattgcttcacatcatagt

SEQ ID NO: 220 - DNA, 50 bp, synthetic construct
  source 1..50: mol_type = other DNA
aagtgactgtactactgttattgcataggactacacaagtacatcatagt

SEQ ID NO: 221 - DNA, 50 bp, synthetic construct
  source 1..50: mol_type = other DNA
aagtgactgtcttaccagtcagagtcttcacgaagatctgacatcatagt

SEQ ID NO: 222 - DNA, 50 bp, synthetic construct
  source 1..50: mol_type = other DNA
aagtgactgtcacgaagatttgcatcccacctctgagacgacatcatagt

SEQ ID NO: 223 - DNA, 50 bp, synthetic construct
  source 1..50: mol_type = other DNA
aagtgactgttcaagggtgatggtcttgccagtgagtgtcacatcatagt

SEQ ID NO: 224 - DNA, 50 bp, synthetic construct
  source 1..50: mol_type = other DNA
aagtgactgttggatctttgctttgacgttctcgatggtgacatcatagt

SEQ ID NO: 225 - DNA, 50 bp, synthetic construct
  source 1..50: mol_type = other DNA
aagtgactgtaaagatcaacctctgctggtcaggaggaatacatcatagt

SEQ ID NO: 226 - DNA, 50 bp, synthetic construct
  source 1..50: mol_type = other DNA
aagtgactgtgtagactctttctggatgttgtagtcagacacatcatagt

SEQ ID NO: 227 - DNA, 50 bp, synthetic construct
  source 1..50: mol_type = other DNA
aagtgactgtctgatcaggaggaatgccttccttatcttgacatcatagt

SEQ ID NO: 228 - DNA, 50 bp, synthetic construct
  source 1..50: mol_type = other DNA
aagtgactgttttctggatgttgtagtcagacagggtacgacatcatagt

SEQ ID NO: 229 - DNA, 50 bp, synthetic construct
  source 1..50: mol_type = other DNA
aagtgactgtctgagacggagtaccaggtgcaaggtggacacatcatagt

SEQ ID NO: 230 - DNA, 50 bp, synthetic construct
  source 1..50: mol_type = other DNA
aagtgactgtcttgccagtgagtgtcttcacgaagatttgacatcatagt

SEQ ID NO: 231 - DNA, 50 bp, synthetic construct
  source 1..50: mol_type = other DNA
aagtgactgtcttccttgtcttggatctttgctttgacgtacatcatagt

SEQ ID NO: 232 - DNA, 50 bp, synthetic construct; identical to SEQ ID NO: 225
  source 1..50: mol_type = other DNA

SEQ ID NO: 233 - DNA, 50 bp, synthetic construct; identical to SEQ ID NO: 226
  source 1..50: mol_type = other DNA

SEQ ID NO: 234 - DNA, 50 bp, synthetic construct
  source 1..50: mol_type = other DNA
aagtgactgtttcacgaagatctgcatcccacctctgagaacatcatagt

SEQ ID NO: 235 - DNA, 50 bp, synthetic construct
  source 1..50: mol_type = other DNA
aagtgactgtcacttcgagagtgatggtcttaccagtcagacatcatagt

SEQ ID NO: 236 - DNA, 50 bp, synthetic construct
  source 1..50: mol_type = other DNA
aagtgactgttcttggatctttgccttgacattctcaatgacatcatagt

SEQ ID NO: 237 - DNA, 50 bp, synthetic construct
  source 1..50: mol_type = other DNA
aagtgactgtacagggtacgaccatcttccagctgttttcacatcatagt

SEQ ID NO: 238 - DNA, 50 bp, synthetic construct
  source 1..50: mol_type = other DNA
aagtgactgtcaaggtggactctttctggatgttgtagtcacatcatagt

SEQ ID NO: 239 - DNA, 50 bp, synthetic construct; identical to SEQ ID NO: 234
  source 1..50: mol_type = other DNA

SEQ ID NO: 240 - DNA, 50 bp, synthetic construct
  source 1..50: mol_type = other DNA
aagtgactgtcacctcgagagtgatggtcttaccagtcagacatcatagt

SEQ ID NO: 241 - DNA, 50 bp, synthetic construct; identical to SEQ ID NO: 236
  source 1..50: mol_type = other DNA

SEQ ID NO: 242 - DNA, 50 bp, synthetic construct
  source 1..50: mol_type = other DNA
aagtgactgtttcacgaagatctgcatcccacctctaagaacatcatagt

SEQ ID NO: 243 - DNA, 50 bp, synthetic construct; identical to SEQ ID NO: 235
  source 1..50: mol_type = other DNA

SEQ ID NO: 244 - DNA, 50 bp, synthetic construct; identical to SEQ ID NO: 236
  source 1..50: mol_type = other DNA

SEQ ID NO: 245 - DNA, 50 bp, synthetic construct; identical to SEQ ID NO: 242
  source 1..50: mol_type = other DNA

SEQ ID NO: 246 - DNA, 50 bp, synthetic construct; identical to SEQ ID NO: 235
  source 1..50: mol_type = other DNA

SEQ ID NO: 247 - DNA, 50 bp, synthetic construct; identical to SEQ ID NO: 236
  source 1..50: mol_type = other DNA

SEQ ID NO: 248 - DNA, 50 bp, synthetic construct
  source 1..50: mol_type = other DNA
aagtgactgtcttaccagtcagggtcttcacgaagatttgacatcatagt

SEQ ID NO: 249 - DNA, 50 bp, synthetic construct
  source 1..50: mol_type = other DNA
aagtgactgtctgatcaggagggatgccttccttatcttgacatcatagt

SEQ ID NO: 250 - DNA, 50 bp, synthetic construct
  source 1..50: mol_type = other DNA
aagtgactgtatcttccagctgtttcccagcaaagatcaaacatcatagt

SEQ ID NO: 251 - DNA, 50 bp, synthetic construct
  source 1..50: mol_type = other DNA
aagtgactgtctcaagcgcaggaccaagtgcagagtggacacatcatagt

SEQ ID NO: 252 - DNA, 50 bp, synthetic construct
  source 1..50: mol_type = other DNA
aagtgactgtatccacaaacaagaactgcgacccaaatccacatcatagt

SEQ ID NO: 253 - DNA, 50 bp, synthetic construct
  source 1..50: mol_type = other DNA
aagtgactgtggcaaatctggcgtgtaattttcaatcctgacatcatagt

SEQ ID NO: 254 - DNA, 50 bp, synthetic construct
  source 1..50: mol_type = other DNA
aagtgactgtccttggtgaatgtcagaggtgtgacattacacatcatagt

SEQ ID NO: 255 - DNA, 50 bp, synthetic construct
  source 1..50: mol_type = other DNA
aagtgactgtaaagatatcatgatgtctgcttgaccctcaacatcatagt

SEQ ID NO: 256 - DNA, 50 bp, synthetic construct
  source 1..50: mol_type = other DNA
aagtgactgtaaaggagagttgtcccgatgatctcccctgacatcatagt

SEQ ID NO: 257 - DNA, 50 bp, synthetic construct
  source 1..50: mol_type = other DNA
aagtgactgtagttgtactctctgaaattgttggtccaccacatcatagt

SEQ ID NO: 258 - DNA, 50 bp, synthetic construct
  source 1..50: mol_type = other DNA
aagtgactgtcaaagccccgatatcagtagaatgggagagacatcatagt

SEQ ID NO: 259 - DNA, 50 bp, synthetic construct
  source 1..50: mol_type = other DNA
aagtgactgtaacatcaccactgaaggtgtagctagggtaacatcatagt

SEQ ID NO: 260 - DNA, 50 bp, synthetic construct
  source 1..50: mol_type = other DNA
aagtgactgtttggatgccatcaatgtcatcctgagctagacatcatagt

SEQ ID NO: 261 - DNA, 50 bp, synthetic construct
  source 1..50: mol_type = other DNA
aagtgactgtctggacaggattttgggaacgtccatatatacatcatagt

SEQ ID NO: 262 - DNA, 50 bp, synthetic construct
  source 1..50: mol_type = other DNA
aagtgactgtaaaggttagcttactgtcacacgcttttggacatcatagt

SEQ ID NO: 263 - DNA, 50 bp, synthetic construct
  source 1..50: mol_type = other DNA
aagtgactgtaaagaacatcacttctccccgaatcgtagtacatcatagt

SEQ ID NO: 264 - DNA, 50 bp, synthetic construct
  source 1..50: mol_type = other DNA
aagtgactgtgaagggatttgtgcgcatgtagaatctgtcacatcatagt

SEQ ID NO: 265 - DNA, 50 bp, synthetic construct
  source 1..50: mol_type = other DNA
aagtgactgtggacttcatctctgtcggcaaattcgtaagacatcatagt

SEQ ID NO: 266 - DNA, 50 bp, synthetic construct
  source 1..50: mol_type = other DNA
aagtgactgtcacattctgtccctgaacagcccagtacttacatcatagt

SEQ ID NO: 267 - DNA, 50 bp, synthetic construct
  source 1..50: mol_type = other DNA
aagtgactgtcaaaggagctgtagatgtccttggggtatcacatcatagt

SEQ ID NO: 268 - DNA, 50 bp, synthetic construct
  source 1..50: mol_type = other DNA
aagtgactgtagcatcgatatgcttcacagttctagggaaacatcatagt

SEQ ID NO: 269 - DNA, 50 bp, synthetic construct
  source 1..50: mol_type = other DNA
aagtgactgtcatcatacctccagtatttgttagcaacaaacatcatagt

SEQ ID NO: 270 - DNA, 50 bp, synthetic construct
  source 1..50: mol_type = other DNA
aagtgactgtgggataacctggatccatagatcgtttataacatcatagt

SEQ ID NO: 271 - DNA, 50 bp, synthetic construct
  source 1..50: mol_type = other DNA
aagtgactgtccaattccaggaaagtcatgtgctatcattacatcatagt

SEQ ID NO: 272 - DNA, 50 bp, synthetic construct
  source 1..50: mol_type = other DNA
aagtgactgtctggagagtcaaaattctcttcgttttaggacatcatagt

SEQ ID NO: 273 - DNA, 50 bp, synthetic construct
  source 1..50: mol_type = other DNA
aagtgactgtcaatttttcctgcagttgaaccagctattaacatcatagt

SEQ ID NO: 274 - DNA, 50 bp, synthetic construct
  source 1..50: mol_type = other DNA
aagtgactgtaaatagacagttcttcaggaaaacaccttcacatcatagt

SEQ ID NO: 275 - DNA, 50 bp, synthetic construct
  source 1..50: mol_type = other DNA
aagtgactgtatattctgtgtatcagtgactctagaggttacatcatagt

SEQ ID NO: 276 - DNA, 50 bp, synthetic construct
  source 1..50: mol_type = other DNA
aagtgactgtactttttgtacccaccatttgtggaactaaacatcatagt

SEQ ID NO: 277 - DNA, 50 bp, synthetic construct
  source 1..50: mol_type = other DNA
aagtgactgtcagtcgtctcatcctggtagtagccgtaggacatcatagt

SEQ ID NO: 278 - DNA, 50 bp, synthetic construct
  source 1..50: mol_type = other DNA
aagtgactgtgttctgcttgtcctggcaggagaacacgagacatcatagt

SEQ ID NO: 279 - DNA, 50 bp, synthetic construct
  source 1..50: mol_type = other DNA
aagtgactgttccacgtggttggcctcgtcggaatacgtgacatcatagt

SEQ ID NO: 280 - DNA, 50 bp, synthetic construct
  source 1..50: mol_type = other DNA
aagtgactgtctctgggggtgtggaccgtgtaatccaacgacatcatagt

SEQ ID NO: 281 - DNA, 50 bp, synthetic construct
  source 1..50: mol_type = other DNA
aagtgactgtccgtgctggctatgaggtcttgttctggagacatcatagt

SEQ ID NO: 282 - DNA, 50 bp, synthetic construct
  source 1..50: mol_type = other DNA
aagtgactgtcaatagacagggatgaggttgtcggtggtgacatcatagt

SEQ ID NO: 283 - DNA, 50 bp, synthetic construct
  source 1..50: mol_type = other DNA
aagtgactgtttgcagctgttccacctcttgaaggctatgacatcatagt

SEQ ID NO: 284 - DNA, 50 bp, synthetic construct
  source 1..50: mol_type = other DNA
aagtgactgtctcatgggtaaaggagtctatgtgctcgggacatcatagt

SEQ ID NO: 285 - DNA, 50 bp, synthetic construct
  source 1..50: mol_type = other DNA
aagtgactgtagactctccacgaggtcggctcgctggatgacatcatagt

SEQ ID NO: 286 - DNA, 50 bp, synthetic construct
  source 1..50: mol_type = other DNA
aagtgactgtcacaccggggatgtggcagtggactcactgacatcatagt

SEQ ID NO: 287 - DNA, 50 bp, synthetic construct
  source 1..50: mol_type = other DNA
aagtgactgtggggttggctggagcatcggttgtcggaatacatcatagt

SEQ ID NO: 288 - DNA, 50 bp, synthetic construct
  source 1..50: mol_type = other DNA
aagtgactgttggacaggaagtgtggtcagaagtgaagggacatcatagt

SEQ ID NO: 289 - DNA, 50 bp, synthetic construct
  source 1..50: mol_type = other DNA
aagtgactgtcaagttcacagtgtagagtccatggctctgacatcatagt

SEQ ID NO: 290 - DNA, 50 bp, synthetic construct
  source 1..50: mol_type = other DNA
aagtgactgtcttcaagccatttcccagaaggtcacctgcacatcatagt

SEQ ID NO: 291 - DNA, 50 bp, synthetic construct
  source 1..50: mol_type = other DNA
aagtgactgttggagacagcgtggaaggcaaagctgacttacatcatagt

SEQ ID NO: 292 - DNA, 50 bp, synthetic construct
  source 1..50: mol_type = other DNA
aagtgactgtacgaaccccaaacctgactccatcatggacacatcatagt

SEQ ID NO: 293 - DNA, 50 bp, synthetic construct
  source 1..50: mol_type = other DNA
aagtgactgtagactggagccatgccactgatgtcacacaacatcatagt

SEQ ID NO: 294 - DNA, 50 bp, synthetic construct
  source 1..50: mol_type = other DNA
aagtgactgttgttagctccgggtccatgtcgggatggagacatcatagt

SEQ ID NO: 295 - DNA, 50 bp, synthetic construct
  source 1..50: mol_type = other DNA
aagtgactgtgagtgaagacggccgagcagacctcaattcacatcatagt

SEQ ID NO: 296 - DNA, 50 bp, synthetic construct
  source 1..50: mol_type = other DNA
aagtgactgtattcctccacgtgtaagcccttgaagacagacatcatagt

SEQ ID NO: 297 - DNA, 50 bp, synthetic construct
  source 1..50: mol_type = other DNA
aagtgactgttggcccacttttcttcatcacaaccttctgacatcatagt

SEQ ID NO: 298 - DNA, 50 bp, synthetic construct
  source 1..50: mol_type = other DNA
aagtgactgtagtcgaagtcaattccttcttgccgcattcacatcatagt

SEQ ID NO: 299 - DNA, 50 bp, synthetic construct
  source 1..50: mol_type = other DNA
aagtgactgtcaaagaattgccatttactacagtgctcctacatcatagt

SEQ ID NO: 300 - DNA, 50 bp, synthetic construct
  source 1..50: mol_type = other DNA
aagtgactgtgtgagtttcctcccctcatcacaggttgagacatcatagt

SEQ ID NO: 301 - DNA, 50 bp, synthetic construct
  source 1..50: mol_type = other DNA
aagtgactgtttacgtcgccgtccagctcgaccaggatggacatcatagt

SEQ ID NO: 302 - DNA, 50 bp, synthetic construct
  source 1..50: mol_type = other DNA
aagtgactgttaggtggcatcgccctcgccctcgccggacacatcatagt

SEQ ID NO: 303 - DNA, 50 bp, synthetic construct
  source 1..50: mol_type = other DNA
aagtgactgtgtggtgcagatgaacttcagggtcagcttgacatcatagt

SEQ ID NO: 304 - DNA, 50 bp, synthetic construct
  source 1..50: mol_type = other DNA
aagtgactgtatgtggtcggggtagcggctgaagcactgcacatcatagt

SEQ ID NO: 305 - DNA, 50 bp, synthetic construct
  source 1..50: mol_type = other DNA
aagtgactgtgcgctcctggacgtagccttcgggcatggcacatcatagt

SEQ ID NO: 306 - DNA, 50 bp, synthetic construct
  source 1..50: mol_type = other DNA
aagtgactgtccctcgaacttcacctcggcgcgggtcttgacatcatagt

SEQ ID NO: 307 - DNA, 50 bp, synthetic construct
  source 1..50: mol_type = other DNA
aagtgactgtagttgtactccagcttgtgccccaggatgtacatcatagt

SEQ ID NO: 308 - DNA, 50 bp, synthetic construct
  source 1..50: mol_type = other DNA
aagtgactgtgctgccgtcctcgatgttgtggcggatcttacatcatagt

SEQ ID NO: 309 - DNA, 50 bp, synthetic construct
  source 1..50: mol_type = other DNA
aagtgactgtttgtcgggcagcagcacggggccgtcgccgacatcatagt

SEQ ID NO: 310 - DNA, 50 bp, synthetic construct
  source 1..50: mol_type = other DNA
aagtgactgtactccagcaggaccatgtgatcgcgcttctacatcatagt

SEQ ID NO: 311 - DNA, 20 bp, synthetic construct
  source 1..20: mol_type = other DNA
acagtcacttcaacactcag

SEQ ID NO: 312 - DNA, 27 bp, synthetic construct
  source 1..27: mol_type = other DNA
tcttgacgagaatgtcttactatgatg

SEQ ID NO: 313 - DNA, 58 bp, synthetic construct
  source 1..58: mol_type = other DNA
agacattctcgtcaagattcacgcttattcgcatgtttagtacgccctctgagtgttg

SEQ ID NO: 314 - DNA, 58 bp, synthetic construct
  source 1..58: mol_type = other DNA
agacattctcgtcaagatacttatactcgccgataaaaacgtcgcacgctgagtgttg

SEQ ID NO: 315 - DNA, 58 bp, synthetic construct
  source 1..58: mol_type = other DNA
agacattctcgtcaagatgtgttttgcgtccgattgtaccgataccacctgagtgttg

SEQ ID NO: 316 - DNA, 58 bp, synthetic construct
  source 1..58: mol_type = other DNA
agacattctcgtcaagatatacctacaccgacatagacgtaagccagcctgagtgttg

SEQ ID NO: 317 - DNA, 58 bp, synthetic construct
  source 1..58: mol_type = other DNA
agacattctcgtcaagatagtcggtgtcttagatcggttcgctctttactgagtgttg

SEQ ID NO: 318 - DNA, 58 bp, synthetic construct
  source 1..58: mol_type = other DNA
agacattctcgtcaagatgccattggtatgctattaaccgctcgactactgagtgttg

SEQ ID NO: 319 - DNA, 58 bp, synthetic construct
  source 1..58: mol_type = other DNA
agacattctcgtcaagattagaacaacggtcgatccacttcgctcacactgagtgttg

SEQ ID NO: 320 - DNA, 58 bp, synthetic construct
  source 1..58: mol_type = other DNA
agacattctcgtcaagatctctgtacggttctatcagattgtcgagtcctgagtgttg

SEQ ID NO: 321 - DNA, 58 bp, synthetic construct
  source 1..58: mol_type = other DNA
agacattctcgtcaagattacggtggtgagaaattggtgtctgatgcgctgagtgttg

SEQ ID NO: 322 - DNA, 58 bp, synthetic construct
  source 1..58: mol_type = other DNA
agacattctcgtcaagatgtgtgtattccgcaatccgctgatacttgtctgagtgttg

SEQ ID NO: 323 - DNA, 58 bp, synthetic construct
  source 1..58: mol_type = other DNA
agacattctcgtcaagattgtggttctgcaagatgtaggtagtggtacctgagtgttg

SEQ ID NO: 324 - DNA, 58 bp, synthetic construct
  source 1..58: mol_type = other DNA
agacattctcgtcaagatggtgtaagagtccgatgcgttgtttgtcctctgagtgttg

SEQ ID NO: 325 - DNA, 58 bp, synthetic construct
  source 1..58: mol_type = other DNA
agacattctcgtcaagatgacaataaccgacgataactccgatacgctctgagtgttg

SEQ ID NO: 326 - DNA, 58 bp, synthetic construct
  source 1..58: mol_type = other DNA
agacattctcgtcaagattgaatggctcgactatggtgtgttcgtttgctgagtgttg

SEQ ID NO: 327 - DNA, 58 bp, synthetic construct
  source 1..58: mol_type = other DNA
agacattctcgtcaagatgttcggttgacactatcacgctttgaatccctgagtgttg

SEQ ID NO: 328 - DNA, 89 bp, synthetic construct
  source 1..89: mol_type = other DNA
tgcgaataagcgtgaaactaacgtcgggaattcgtcgtgactagcaactaacgtcgggaattcgtcgtgactagcagggcgtactaaac

SEQ ID NO: 329 - DNA, 89 bp, synthetic construct
  source 1..89: mol_type = other DNA
tcggcgagtataagtagacggtcgagtatatggcgaatcgttaagagacggtcgagtatatggcgaatcgttaagcgtgcgacgttttt

SEQ ID NO: 330 - DNA, 89 bp, synthetic construct
  source 1..89: mol_type = other DNA
tcggacgcaaaacacacaactaggtcggcatggacgctatatgagacaactaggtcggcatggacgctatatgaggtggtatcggtaca

SEQ ID NO: 331 - DNA, 89 bp, synthetic construct
  source 1..89: mol_type = other DNA
tgtcggtgtaggtattcgtccactcgtctatttgcggttgcgtgttcgtccactcgtctatttgcggttgcgtgtgctggcttacgtct

SEQ ID NO: 332 - DNA, 89 bp, synthetic construct
  source 1..89: mol_type = other DNA
tctaagacaccgactaaccgaacgtactgatattaccgtcgagcaaaccgaacgtactgatattaccgtcgagcataaagagcgaaccg

SEQ ID NO: 333 - DNA, 89 bp, synthetic construct
  source 1..89: mol_type = other DNA
tagcataccaatggcctagtagcttagcgatacggtaggtctagtctagtagcttagcgatacggtaggtctagttagtcgagcggtta

SEQ ID NO: 334 - DNA, 89 bp, synthetic construct
  source 1..89: mol_type = other DNA
tcgaccgttgttctaccttaactagagcgatacgagcctaccgaaccttaactagagcgatacgagcctaccgaatgtgagcgaagtgg

SEQ ID NO: 335 - DNA, 89 bp, synthetic construct
  source 1..89: mol_type = other DNA
tagaaccgtacagagtgacctcaaccgttatccagcgaaacaagatgacctcaaccgttatccagcgaaacaagagactcgacaatctg

SEQ ID NO: 336 - DNA, 89 bp, synthetic construct
  source 1..89: mol_type = other DNA
tttctcaccaccgtatgcttctatccgtgataccgacttcgttagtgcttctatccgtgataccgacttcgttagcgcatcagacacca

SEQ ID NO: 337 - DNA, 89 bp, synthetic construct
  source 1..89: mol_type = other DNA
ttgcggaatacacacctaacaagcgactcattggttccagatgcactaacaagcgactcattggttccagatgcaacaagtatcagcgg

SEQ ID NO: 338 - DNA, 89 bp, synthetic construct
  source 1..89: mol_type = other DNA
tcttgcagaaccacacaactaggagcttcatcacgaccaaagactcaactaggagcttcatcacgaccaaagactgtaccactacctac

SEQ ID NO: 339 - DNA, 89 bp, synthetic construct
  source 1..89: mol_type = other DNA
tcggactcttacacctaggttcactgctcatacgctcgaatcttctaggttcactgctcatacgctcgaatcttcaggacaaacaacgc

SEQ ID NO: 340 - DNA, 89 bp, synthetic construct
  source 1..89: mol_type = other DNA
tcgtcggttattgtctcgcaaacggagatatatgatcggcaacactcgcaaacggagatatatgatcggcaacacagcgtatcggagtt

SEQ ID NO: 341 - DNA, 89 bp, synthetic construct
  source 1..89: mol_type = other DNA
tagtcgagccattcatgtaacgtctgaggattcttgtccgcacaatgtaacgtctgaggattcttgtccgcacaacaaacgaacacacc

SEQ ID NO: 342 - DNA, 89 bp, synthetic construct
  source 1..89: mol_type = other DNA
tagtgtcaaccgaaccatagtagaaggcgatctccagtgactcatcatagtagaaggcgatctccagtgactcatggattcaaagcgtg

SEQ ID NO: 343 - DNA, 89 bp, synthetic construct
  source 1..89: mol_type = other DNA
ttcccgacgttagtttcacgcttattcgcatgtttagtacgcccttcacgcttattcgcatgtttagtacgccctgctagtcacgacga

SEQ ID NO: 344 - DNA, 89 bp, synthetic construct
  source 1..89: mol_type = other DNA
tatactcgaccgtctacttatactcgccgataaaaacgtcgcacgacttatactcgccgataaaaacgtcgcacgcttaacgattcgcc

SEQ ID NO: 345 - DNA, 89 bp, synthetic construct
  source 1..89: mol_type = other DNA
tgccgacctagttgtgtgttttgcgtccgattgtaccgataccacgtgttttgcgtccgattgtaccgataccacctcatatagcgtcc

SEQ ID NO: 346 - DNA, 89 bp, synthetic construct
  source 1..89: mol_type = other DNA
tagacgagtggacgaatacctacaccgacatagacgtaagccagcatacctacaccgacatagacgtaagccagcacacgcaaccgcaa

SEQ ID NO: 347 - DNA, 89 bp, synthetic construct
  source 1..89: mol_type = other DNA
tcagtacgttcggttagtcggtgtcttagatcggttcgctctttaagtcggtgtcttagatcggttcgctctttatgctcgacggtaat

SEQ ID NO: 348 - DNA, 89 bp, synthetic construct
  source 1..89: mol_type = other DNA
tcgctaagctactaggccattggtatgctattaaccgctcgactagccattggtatgctattaaccgctcgactaactagacctaccgt

SEQ ID NO: 349 - DNA, 89 bp, synthetic construct
  source 1..89: mol_type = other DNA
tcgctctagttaaggtagaacaacggtcgatccacttcgctcacatagaacaacggtcgatccacttcgctcacattcggtaggctcgt

SEQ ID NO: 350 - DNA, 89 bp, synthetic construct
  source 1..89: mol_type = other DNA
taacggttgaggtcactctgtacggttctatcagattgtcgagtcctctgtacggttctatcagattgtcgagtctcttgtttcgctgg

SEQ ID NO: 351 - DNA, 89 bp, synthetic construct
  source 1..89: mol_type = other DNA
tcacggatagaagcatacggtggtgagaaattggtgtctgatgcgtacggtggtgagaaattggtgtctgatgcgctaacgaagtcggt

SEQ ID NO: 352 - DNA, 89 bp, synthetic construct
  source 1..89: mol_type = other DNA
tgagtcgcttgttaggtgtgtattccgcaatccgctgatacttgtgtgtgtattccgcaatccgctgatacttgttgcatctggaacca

SEQ ID NO: 353 - DNA, 89 bp, synthetic construct
  source 1..89: mol_type = other DNA
tgaagctcctagttgtgtggttctgcaagatgtaggtagtggtactgtggttctgcaagatgtaggtagtggtacagtctttggtcgtg

SEQ ID NO: 354 - DNA, 89 bp, synthetic construct
  source 1..89: mol_type = other DNA
tgagcagtgaacctaggtgtaagagtccgatgcgttgtttgtcctggtgtaagagtccgatgcgttgtttgtcctgaagattcgagcgt

SEQ ID NO: 355 - DNA, 89 bp, synthetic construct
  source 1..89: mol_type = other DNA
tatctccgtttgcgagacaataaccgacgataactccgatacgctgacaataaccgacgataactccgatacgctgtgttgccgatcat

SEQ ID NO: 356 - DNA, 89 bp, synthetic construct
  source 1..89: mol_type = other DNA
tcctcagacgttacatgaatggctcgactatggtgtgttcgtttgtgaatggctcgactatggtgtgttcgtttgttgtgcggacaaga

SEQ ID NO: 357 - DNA, 89 bp, synthetic construct
  source 1..89: mol_type = other DNA
tcgccttctactatggttcggttgacactatcacgctttgaatccgttcggttgacactatcacgctttgaatccatgagtcactggag

SEQ ID NO: 358 - DNA, 14 bp, synthetic construct
  source 1..14: mol_type = other DNA
tcggtatcacggat

SEQ ID NO: 359 - DNA, 16 bp, synthetic construct
  source 1..16: mol_type = other DNA
agacaccaatttctca

SEQ ID NO: 360 - DNA, 15 bp, synthetic construct
  source 1..15: mol_type = other DNA
gctcgtatcgctcta

SEQ ID NO: 361 - DNA, 14 bp, synthetic construct
  source 1..14: mol_type = other DNA
agtggatcgaccgt

SEQ ID NO: 362 - DNA, 14 bp, synthetic construct
  source 1..14: mol_type = other DNA
cgacgaattcccga

SEQ ID NO: 363 - DNA, 17 bp, synthetic construct
  source 1..17: mol_type = other DNA
actaaacatgcgaataa

SEQ ID NO: 364 - DNA, 15 bp, synthetic construct
  source 1..15: mol_type = other DNA
gaaccaatgagtcgc

SEQ ID NO: 365 - DNA, 14 bp, synthetic construct
  source 1..14: mol_type = other DNA
agcggattgcggaa

SEQ ID NO: 366 - DNA, 16 bp, synthetic construct
  source 1..16: mol_type = other DNA
ctaccgtatcgctaag

SEQ ID NO: 367 - DNA, 16 bp, synthetic construct
  source 1..16: mol_type = other DNA
gcggttaatagcatac

SEQ ID NO: 368 - DNA, 16 bp, synthetic construct
  source 1..16: mol_type = other DNA
ggtaatatcagtacgt

SEQ ID NO: 369 - DNA, 15 bp, synthetic construct
  source 1..15: mol_type = other DNA
gaaccgatctaagac

SEQ ID NO: 370 - DNA, 15 bp, synthetic construct
  source 1..15: mol_type = other DNA
gacaagaatcctcag

SEQ ID NO: 371 - DNA, 15 bp, synthetic construct
  source 1..15: mol_type = other DNA
cacaccatagtcgag

SEQ ID NO: 372 - DNA, 12 bp, synthetic construct
  source 1..12: mol_type = other DNA
gcgtccatgccg

SEQ ID NO: 373 - DNA, 14 bp, synthetic construct
  source 1..14: mol_type = other DNA
cggtacaatcggac

SEQ ID NO: 374 - DNA, 15 bp, synthetic construct
  source 1..15: mol_type = other DNA
ctggagatcgccttc

SEQ ID NO: 375 - DNA, 16 bp, synthetic construct
  source 1..16: mol_type = other DNA
aaagcgtgatagtgtc

SEQ ID NO: 376 - DNA, 14 bp, synthetic construct
  source 1..14: mol_type = other DNA
agcgtatgagcagt

SEQ ID NO: 377 - DNA, 13 bp, synthetic construct
  source 1..13: mol_type = other DNA
caacgcatcggac

SEQ ID NO: 378 - DNA, 20 bp, synthetic construct
  source 1..20: mol_type = other DNA
tcacgacgaattcccgacgt

SEQ ID NO: 379 - DNA, 20 bp, synthetic construct
  source 1..20: mol_type = other DNA
cgattcgccatatactcgac

SEQ ID NO: 380 - DNA, 20 bp, synthetic construct
  source 1..20: mol_type = other DNA
atagcgtccatgccgaccta

SEQ ID NO: 381 - DNA, 20 bp, synthetic construct
  source 1..20: mol_type = other DNA
caaccgcaaatagacgagtg

SEQ ID NO: 382 - DNA, 20 bp, synthetic construct
  source 1..20: mol_type = other DNA
gacggtaatatcagtacgtt

SEQ ID NO: 383 - DNA, 20 bp, synthetic construct
  source 1..20: mol_type = other DNA
acctaccgtatcgctaagct

SEQ ID NO: 384 - DNA, 20 bp, synthetic construct
  source 1..20: mol_type = other DNA
taggctcgtatcgctctagt

SEQ ID NO: 385 - DNA, 20 bp, synthetic construct
  source 1..20: mol_type = other DNA
tttcgctggataacggttga

SEQ ID NO: 386 - DNA, 20 bp, synthetic construct
  source 1..20: mol_type = other DNA
gaagtcggtatcacggatag

SEQ ID NO: 387 - DNA, 20 bp, synthetic construct
  source 1..20: mol_type = other DNA
ctggaaccaatgagtcgctt

SEQ ID NO: 388 - DNA, 20 bp, synthetic construct
  source 1..20: mol_type = other DNA
ttggtcgtgatgaagctcct

SEQ ID NO: 389 - DNA, 20 bp, synthetic construct
  source 1..20: mol_type = other DNA
ttcgagcgtatgagcagtga

SEQ ID NO: 390 - DNA, 20 bp, synthetic construct
  source 1..20: mol_type = other DNA
gccgatcatatatctccgtt

SEQ ID NO: 391 - DNA, 20 bp, synthetic construct
  source 1..20: mol_type = other DNA
cggacaagaatcctcagacg

SEQ ID NO: 392 - DNA, 20 bp, synthetic construct
  source 1..20: mol_type = other DNA
tcactggagatcgccttcta

SEQ ID NO: 393 - DNA, 30 bp, synthetic construct
  source 1..30: mol_type = other DNA
aactaacgtcgggaattcgtcgtgactagc

SEQ ID NO: 394 - DNA, 30 bp, synthetic construct
  source 1..30: mol_type = other DNA
agacggtcgagtatatggcgaatcgttaag

SEQ ID NO: 395 - DNA, 30 bp, synthetic construct
  source 1..30: mol_type = other DNA
acaactaggtcggcatggacgctatatgag

SEQ ID NO: 396 - DNA, 30 bp, synthetic construct
  source 1..30: mol_type = other DNA
tcgtccactcgtctatttgcggttgcgtgt

SEQ ID NO: 397 - DNA, 30 bp, synthetic construct
  source 1..30: mol_type = other DNA
aaccgaacgtactgatattaccgtcgagca

SEQ ID NO: 398 - DNA, 30 bp, synthetic construct
  source 1..30: mol_type = other DNA
ctagtagcttagcgatacggtaggtctagt

SEQ ID NO: 399 - DNA, 30 bp, synthetic construct
  source 1..30: mol_type = other DNA
ccttaactagagcgatacgagcctaccgaa

SEQ ID NO: 400 - DNA, 30 bp, synthetic construct
  source 1..30: mol_type = other DNA
tgacctcaaccgttatccagcgaaacaaga

SEQ ID NO: 401 - DNA, 30 bp, synthetic construct
  source 1..30: mol_type = other DNA
tgcttctatccgtgataccgacttcgttag

SEQ ID NO: 402 - DNA, 30 bp, synthetic construct
  source 1..30: mol_type = other DNA
ctaacaagcgactcattggttccagatgca

SEQ ID NO: 403 - DNA, 30 bp, synthetic construct
  source 1..30: mol_type = other DNA
caactaggagcttcatcacgaccaaagact

SEQ ID NO: 404 - DNA, 30 bp, synthetic construct
  source 1..30: mol_type = other DNA
taggttcactgctcatacgctcgaatcttc

SEQ ID NO: 405 - DNA, 30 bp, synthetic construct
  source 1..30: mol_type = other DNA
tcgcaaacggagatatatgatcggcaacac

SEQ ID NO: 406 - DNA, 30 bp, synthetic construct
  source 1..30: mol_type = other DNA
tgtaacgtctgaggattcttgtccgcacaa

SEQ ID NO: 407 - DNA, 30 bp, synthetic construct
  source 1..30: mol_type = other DNA
catagtagaaggcgatctccagtgactcat

SEQ ID NO: 408 - DNA, 30 bp, synthetic construct
  source 1..30: mol_type = other DNA
tcacgcttattcgcatgtttagtacgccct

SEQ ID NO: 409 - DNA, 30 bp, synthetic construct
  source 1..30: mol_type = other DNA
acttatactcgccgataaaaacgtcgcacg

SEQ ID NO: 410 - DNA, 30 bp, synthetic construct
  source 1..30: mol_type = other DNA
gtgttttgcgtccgattgtaccgataccac

SEQ ID NO: 411 - DNA, 30 bp, synthetic construct
  source 1..30: mol_type = other DNA
atacctacaccgacatagacgtaagccagc

SEQ ID NO: 412 - DNA, 30 bp, synthetic construct
  source 1..30: mol_type = other DNA
agtcggtgtcttagatcggttcgctcttta

SEQ ID NO: 413 - DNA, 30 bp, synthetic construct
  source 1..30: mol_type = other DNA
gccattggtatgctattaaccgctcgacta

SEQ ID NO: 414 - DNA, 30 bp, synthetic construct
  source 1..30: mol_type = other DNA
tagaacaacggtcgatccacttcgctcaca

SEQ ID NO: 415 - DNA, 30 bp, synthetic construct
  source 1..30: mol_type = other DNA
ctctgtacggttctatcagattgtcgagtc

SEQ ID NO: 416 - DNA, 30 bp, synthetic construct
  source 1..30: mol_type = other DNA
tacggtggtgagaaattggtgtctgatgcg

SEQ ID NO: 417 - DNA, 30 bp, synthetic construct
  source 1..30: mol_type = other DNA
gtgtgtattccgcaatccgctgatacttgt

SEQ ID NO: 418 - DNA, 30 bp, synthetic construct
  source 1..30: mol_type = other DNA
tgtggttctgcaagatgtaggtagtggtac

SEQ ID NO: 419 - DNA, 30 bp, synthetic construct
  source 1..30: mol_type = other DNA
ggtgtaagagtccgatgcgttgtttgtcct

SEQ ID NO: 420 - DNA, 30 bp, synthetic construct
  source 1..30: mol_type = other DNA
gacaataaccgacgataactccgatacgct

SEQ ID NO: 421 - DNA, 30 bp, synthetic construct
  source 1..30: mol_type = other DNA
tgaatggctcgactatggtgtgttcgtttg

SEQ ID NO: 422 - DNA, 30 bp, synthetic construct
  source 1..30: mol_type = other DNA
gttcggttgacactatcacgctttgaatcc

SEQ ID NO: 423 - DNA, 90 bp, synthetic construct
  source 1..90: mol_type = other DNA
tgcgaataagcgtgaaactaacgtcgggaattcgtcgtgactagcaactaacgtcgggaattcgtcgtgactagcagggcgtactaaaca

SEQ ID NO: 424 - DNA, 90 bp, synthetic construct
  source 1..90: mol_type = other DNA
tcggcgagtataagtagacggtcgagtatatggcgaatcgttaagagacggtcgagtatatggcgaatcgttaagcgtgcgacgttttta

SEQ ID NO: 425 - DNA, 90 bp, synthetic construct
  source 1..90: mol_type = other DNA
tcggacgcaaaacacacaactaggtcggcatggacgctatatgagacaactaggtcggcatggacgctatatgaggtggtatcggtacaa

SEQ ID NO: 426 - DNA, 90 bp, synthetic construct
  source 1..90: mol_type = other DNA
tgtcggtgtaggtattcgtccactcgtctatttgcggttgcgtgttcgtccactcgtctatttgcggttgcgtgtgctggcttacgtcta

SEQ ID NO: 427 - DNA, 90 bp, synthetic construct
  source 1..90: mol_type = other DNA
tctaagacaccgactaaccgaacgtactgatattaccgtcgagcaaaccgaacgtactgatattaccgtcgagcataaagagcgaaccga

SEQ ID NO: 428 - DNA, 90 bp, synthetic construct
  source 1..90: mol_type = other DNA
tagcataccaatggcctagtagcttagcgatacggtaggtctagtctagtagcttagcgatacggtaggtctagttagtcgagcggttaa

SEQ ID NO: 429 - DNA, 90 bp, synthetic construct
  source 1..90: mol_type = other DNA
tcgaccgttgttctaccttaactagagcgatacgagcctaccgaaccttaactagagcgatacgagcctaccgaatgtgagcgaagtgga

SEQ ID NO: 430 - DNA, 90 bp, synthetic construct
  source 1..90: mol_type = other DNA
tagaaccgtacagagtgacctcaaccgttatccagcgaaacaagatgacctcaaccgttatccagcgaaacaagagactcgacaatctga

SEQ ID NO: 431 - DNA, 90 bp, synthetic construct
  source 1..90: mol_type = other DNA
tttctcaccaccgtatgcttctatccgtgataccgacttcgttagtgcttctatccgtgataccgacttcgttagcgcatcagacaccaa

SEQ ID NO: 432 - DNA, 90 bp, synthetic construct
  source 1..90: mol_type = other DNA
ttgcggaatacacacctaacaagcgactcattggttccagatgcactaacaagcgactcattggttccagatgcaacaagtatcagcgga

SEQ ID NO: 433 - DNA, 90 bp, synthetic construct
  source 1..90: mol_type = other DNA
tcttgcagaaccacacaactaggagcttcatcacgaccaaagactcaactaggagcttcatcacgaccaaagactgtaccactacctaca

SEQ ID NO: 434 - DNA, 90 bp, synthetic construct
  source 1..90: mol_type = other DNA
tcggactcttacacctaggttcactgctcatacgctcgaatcttctaggttcactgctcatacgctcgaatcttcaggacaaacaacgca

SEQ ID NO: 435 - DNA, 90 bp, synthetic construct
  source 1..90: mol_type = other DNA
tcgtcggttattgtctcgcaaacggagatatatgatcggcaacactcgcaaacggagatatatgatcggcaacacagcgtatcggagtta

SEQ ID NO: 436 - DNA, 90 bp, synthetic construct
  source 1..90: mol_type = other DNA
tagtcgagccattcatgtaacgtctgaggattcttgtccgcacaatgtaacgtctgaggattcttgtccgcacaacaaacgaacacacca

SEQ ID NO: 437 - DNA, 90 bp, synthetic construct
  source 1..90: mol_type = other DNA
tagtgtcaaccgaaccatagtagaaggcgatctccagtgactcatcatagtagaaggcgatctccagtgactcatggattcaaagcgtga

SEQ ID NO: 438 - DNA, 90 bp, synthetic construct
  source 1..90: mol_type = other DNA
ttcccgacgttagtttcacgcttattcgcatgtttagtacgcccttcacgcttattcgcatgtttagtacgccctgctagtcacgacgaa

SEQ ID NO: 439 - DNA, 90 bp, synthetic construct
  source 1..90: mol_type = other DNA
tatactcgaccgtctacttatactcgccgataaaaacgtcgcacgacttatactcgccgataaaaacgtcgcacgcttaacgattcgcca

SEQ ID NO: 440 - DNA, 90 bp, synthetic construct
  source 1..90: mol_type = other DNA
tgccgacctagttgtgtgttttgcgtccgattgtaccgataccacgtgttttgcgtccgattgtaccgataccacctcatatagcgtcca

SEQ ID NO: 441 - DNA, 90 bp, synthetic construct
  source 1..90: mol_type = other DNA
tagacgagtggacgaatacctacaccgacatagacgtaagccagcatacctacaccgacatagacgtaagccagcacacgcaaccgcaaa

SEQ ID NO: 442 - DNA, 90 bp, synthetic construct
  source 1..90: mol_type = other DNA
tcagtacgttcggttagtcggtgtcttagatcggttcgctctttaagtcggtgtcttagatcggttcgctctttatgctcgacggtaata

SEQ ID NO: 443 - DNA, 90 bp, synthetic construct
  source 1..90: mol_type = other DNA
tcgctaagctactaggccattggtatgctattaaccgctcgactagccattggtatgctattaaccgctcgactaactagacctaccgta

SEQ ID NO: 444 - DNA, 90 bp, synthetic construct
  source 1..90: mol_type = other DNA
tcgctctagttaaggtagaacaacggtcgatccacttcgctcacatagaacaacggtcgatccacttcgctcacattcggtaggctcgta

SEQ ID NO: 445 - DNA, 90 bp, synthetic construct
  source 1..90: mol_type = other DNA
taacggttgaggtcactctgtacggttctatcagattgtcgagtcctctgtacggttctatcagattgtcgagtctcttgtttcgctgga

SEQ ID NO: 446 - DNA, 90 bp, synthetic construct
  source 1..90: mol_type = other DNA
tcacggatagaagcatacggtggtgagaaattggtgtctgatgcgtacggtggtgagaaattggtgtctgatgcgctaacgaagtcggta

SEQ ID NO: 447 - DNA, 90 bp, synthetic construct
  source 1..90: mol_type = other DNA
tgagtcgcttgttaggtgtgtattccgcaatccgctgatacttgtgtgtgtattccgcaatccgctgatacttgttgcatctggaaccaa

SEQ ID NO: 448 - DNA, 90 bp, synthetic construct
  source 1..90: mol_type = other DNA
tgaagctcctagttgtgtggttctgcaagatgtaggtagtggtactgtggttctgcaagatgtaggtagtggtacagtctttggtcgtga

SEQ ID NO: 449 - DNA, 90 bp, synthetic construct
  source 1..90: mol_type = other DNA
tgagcagtgaacctaggtgtaagagtccgatgcgttgtttgtcctggtgtaagagtccgatgcgttgtttgtcctgaagattcgagcgta

SEQ ID NO: 450 - DNA, 90 bp, synthetic construct
  source 1..90: mol_type = other DNA
tatctccgtttgcgagacaataaccgacgataactccgatacgctgacaataaccgacgataactccgatacgctgtgttgccgatcata

SEQ ID NO: 451 - DNA, 90 bp, synthetic construct
  source 1..90: mol_type = other DNA
tcctcagacgttacatgaatggctcgactatggtgtgttcgtttgtgaatggctcgactatggtgtgttcgtttgttgtgcggacaagaa

SEQ ID NO: 452 - DNA, 90 bp, synthetic construct
  source 1..90: mol_type = other DNA
tcgccttctactatggttcggttgacactatcacgctttgaatccgttcggttgacactatcacgctttgaatccatgagtcactggaga

SEQ ID NO: 453 - DNA, 109 bp, synthetic construct
  source 1..109: mol_type = other DNA
agacattctcgtcaagattcacgcttattcgcatgtttagtacgccctctgagtgttgaagtgactgtnnnnnnnnnnnnnnnnnnnnnnnnnnnnnnacatcatagta

SEQ ID NO: 454 - DNA, 109 bp, synthetic construct
  source 1..109: mol_type = other DNA
agacattctcgtcaagatacttatactcgccgataaaaacgtcgcacgctgagtgttgaagtgactgtnnnnnnnnnnnnnnnnnnnnnnnnnnnnnnacatcatagta

SEQ ID NO: 455 - DNA, 109 bp, synthetic construct
  source 1..109: mol_type = other DNA
agacattctcgtcaagatgtgttttgcgtccgattgtaccgataccacctgagtgttgaagtgactgtnnnnnnnnnnnnnnnnnnnnnnnnnnnnnnacatcatagta

SEQ ID NO: 456 - DNA, 109 bp, synthetic construct
  source 1..109: mol_type = other DNA
agacattctcgtcaagatatacctacaccgacatagacgtaagccagcctgagtgttgaagtgactgtnnnnnnnnnnnnnnnnnnnnnnnnnnnnnnacatcatagta

SEQ ID NO: 457 - DNA, 109 bp, synthetic construct
  source 1..109: mol_type = other DNA
agacattctcgtcaagatagtcggtgtcttagatcggttcgctctttactgagtgttgaagtgactgtnnnnnnnnnnnnnnnnnnnnnnnnnnnnnnacatcatagta

SEQ ID NO: 458 - DNA, 109 bp, synthetic construct
  source 1..109: mol_type = other DNA
agacattctcgtcaagatgccattggtatgctattaaccgctcgactactgagtgttgaagtgactgtnnnnnnnnnnnnnnnnnnnnnnnnnnnnnnacatcatagta

SEQ ID NO: 459 - DNA, 109 bp, synthetic construct
  source 1..109: mol_type = other DNA
agacattctcgtcaagattagaacaacggtcgatccacttcgctcacactgagtgttgaagtgactgtnnnnnnnnnnnnnnnnnnnnnnnnnnnnnnacatcatagta

SEQ ID NO: 460 - DNA, 109 bp, synthetic construct
  source 1..109: mol_type = other DNA
agacattctcgtcaagatctctgtacggttctatcagattgtcgagtcctgagtgttgaagtgactgtnnnnnnnnnnnnnnnnnnnnnnnnnnnnnnacatcatagta

SEQ ID NO: 461 - DNA, 109 bp, synthetic construct
  source 1..109: mol_type = other DNA
agacattctcgtcaagattacggtggtgagaaattggtgtctgatgcgctgagtgttgaagtgactgtnnnnnnnnnnnnnnnnnnnnnnnnnnnnnnacatcatagta

SEQ ID NO: 462 - DNA, 109 bp, synthetic construct
  source 1..109: mol_type = other DNA
agacattctcgtcaagatgtgtgtattccgcaatccgctgatacttgtctgagtgttgaagtgactgtnnnnnnnnnnnnnnnnnnnnnnnnnnnnnnacatcatagta

SEQ ID NO: 463 - DNA, 109 bp, synthetic construct
  source 1..109: mol_type = other DNA
agacattctcgtcaagattgtggttctgcaagatgtaggtagtggtacctgagtgttgaagtgactgtnnnnnnnnnnnnnnnnnnnnnnnnnnnnnnacatcatagta

SEQ ID NO: 464 - DNA, 109 bp, synthetic construct
  source 1..109: mol_type = other DNA
agacattctcgtcaagatggtgtaagagtccgatgcgttgtttgtcctctgagtgttgaagtgactgtnnnnnnnnnnnnnnnnnnnnnnnnnnnnnnacatcatagta

SEQ ID NO: 465 - DNA, 109 bp, synthetic construct
  source 1..109: mol_type = other DNA
agacattctcgtcaagatgacaataaccgacgataactccgatacgctctgagtgttgaagtgactgtnnnnnnnnnnnnnnnnnnnnnnnnnnnnnnacatcatagta

SEQ ID NO: 466 - DNA, 109 bp, synthetic construct
  source 1..109: mol_type = other DNA
agacattctcgtcaagattgaatggctcgactatggtgtgttcgtttgctgagtgttgaagtgactgtnnnnnnnnnnnnnnnnnnnnnnnnnnnnnnacatcatagta

SEQ ID NO: 467 - DNA, 109 bp, synthetic construct
  source 1..109: mol_type = other DNA
agacattctcgtcaagatgttcggttgacactatcacgctttgaatccctgagtgttgaagtgactgtnnnnnnnnnnnnnnnnnnnnnnnnnnnnnnacatcatagta

SEQ ID NO: 468 - DNA, 17 bp, synthetic construct
  source 1..17: mol_type = other DNA
caccccggtgaacagct

SEQ ID NO: 469 - DNA, 17 bp, synthetic construct
  source 1..17: mol_type = other DNA
ggccagggcacgggcag

SEQ ID NO: 470 - DNA, 17 bp, synthetic construct
  source 1..17: mol_type = other DNA
tcagggtggtcacgagg

SEQ ID NO: 471 - DNA, 17 bp, synthetic construct
  source 1..17: mol_type = other DNA
tgaagaagtcgtgctgc

SEQ ID NO: 472 - DNA, 17 bp, synthetic construct
  source 1..17: mol_type = other DNA
tgccgtcgtccttgaag

SEQ ID NO: 473 - DNA, 17 bp, synthetic construct
  source 1..17: mol_type = other DNA
gctcgatgcggttcacc

SEQ ID NO: 474 - DNA, 17 bp, synthetic construct
  source 1..17: mol_type = other DNA
cttgaagtcgatgccct

SEQ ID NO: 475 - DNA, 17 bp, synthetic construct
  source 1..17: mol_type = other DNA
atagacgttgtggctgt

SEQ ID NO: 476 - DNA, 17 bp, synthetic construct
  source 1..17: mol_type = other DNA
ttctgcttgtcggccat

SEQ ID NO: 477 - DNA, 17 bp, synthetic construct
  source 1..17: mol_type = other DNA
agttcaccttgatgccg

SEQ ID NO: 478 - DNA, 17 bp, synthetic construct
  source 1..17: mol_type = other DNA
tctgctggtagtggtcg

SEQ ID NO: 479 - DNA, 17 bp, synthetic construct
  source 1..17: mol_type = other DNA
actgggtgctcaggtag

SEQ ID NO: 480 - DNA, 17 bp, synthetic construct
  source 1..17: mol_type = other DNA
ggggtctttgctcaggg

SEQ ID NO: 481 - DNA, 17 bp, synthetic construct
  source 1..17: mol_type = other DNA
gagagtgatcccggcgg

SEQ ID NO: 482 - DNA, 17 bp, synthetic construct
  source 1..17: mol_type = other DNA
ttgtacagctcgtccat

SEQ ID NO: 483 - DNA, 20 bp, synthetic construct
  source 1..20: mol_type = other DNA
ctcttgccaatgcacgaagg

SEQ ID NO: 484 - DNA, 20 bp, synthetic construct
  source 1..20: mol_type = other DNA
tatgtccattagcacctctg

SEQ ID NO: 485 - DNA, 20 bp, synthetic construct
  source 1..20: mol_type = other DNA
tgggatggtcaaattccttc

SEQ ID NO: 486 - DNA, 20 bp, synthetic construct
  source 1..20: mol_type = other DNA
tcagatactccatgccactg

SEQ ID NO: 487 - DNA, 20 bp, synthetic construct
  source 1..20: mol_type = other DNA
cggtgtatgaatctcttggt

SEQ ID NO: 488 - DNA, 20 bp, synthetic construct
  source 1..20: mol_type = other DNA
ccattgtagatcttcttgga

SEQ ID NO: 489 - DNA, 20 bp, synthetic construct
  source 1..20: mol_type = other DNA
tggcatcttggcgatacgtc

SEQ ID NO: 490 - DNA, 20 bp, synthetic construct
  source 1..20: mol_type = other DNA
aaggaccacacatcgctctt

SEQ ID NO: 491 - DNA, 20 bp, synthetic construct
  source 1..20: mol_type = other DNA
ataaatctcgctgttctcca

SEQ ID NO: 492 - DNA, 20 bp, synthetic construct
  source 1..20: mol_type = other DNA
ccgcagctctgtaaaacttg

SEQ ID NO: 493 - DNA, 20 bp, synthetic construct
  source 1..20: mol_type = other DNA
tcagtgtgttctccaaatct

SEQ ID NO: 494 - DNA, 20 bp, synthetic construct
  source 1..20: mol_type = other DNA
acatagcgtccagcaggatg

SEQ ID NO: 495 - DNA, 20 bp, synthetic construct
  source 1..20: mol_type = other DNA
taggggttgtggaagggcag

SEQ ID NO: 496 - DNA, 20 bp, synthetic construct
  source 1..20: mol_type = other DNA
gcacagagaaggggagggac

SEQ ID NO: 497 - DNA, 20 bp, synthetic construct
  source 1..20: mol_type = other DNA
ctgctttcaaggtgatgcta

SEQ ID NO: 498 - DNA, 20 bp, synthetic construct
  source 1..20: mol_type = other DNA
tacagccagagaatctagac

SEQ ID NO: 499 - DNA, 20 bp, synthetic construct
  source 1..20: mol_type = other DNA
cttggagcatctgatctaga

SEQ ID NO: 500 - DNA, 20 bp, synthetic construct
  source 1..20: mol_type = other DNA
acaactgagtgtgagagtct

SEQ ID NO: 501 - DNA, 20 bp, synthetic construct
  source 1..20: mol_type = other DNA
caaagaacctaaaggtgtcc

SEQ ID NO: 502 - DNA, 20 bp, synthetic construct
  source 1..20: mol_type = other DNA
cacagttggggaggaggaag

SEQ ID NO: 503 - DNA, 20 bp, synthetic construct
  source 1..20: mol_type = other DNA
aaaggtgggagatcgtccat

SEQ ID NO: 504 - DNA, 20 bp, synthetic construct
  source 1..20: mol_type = other DNA
aaggtgagggattggctcac

SEQ ID NO: 505 - DNA, 20 bp, synthetic construct
  source 1..20: mol_type = other DNA
agagtccacactctgtactc

SEQ ID NO: 506 - DNA, 20 bp, synthetic construct
  source 1..20: mol_type = other DNA
gtaaatttctcatgggcagc

SEQ ID NO: 507 - DNA, 20 bp, synthetic construct
  source 1..20: mol_type = other DNA
acagcttggatcacactttt

SEQ ID NO: 508 - DNA, 20 bp, synthetic construct
  source 1..20: mol_type = other DNA
gtcagtttctggcagttctc

SEQ ID NO: 509 - DNA, 20 bp, synthetic construct
  source 1..20: mol_type = other DNA
ctgctgggcacagatgattt

SEQ ID NO: 510 - DNA, 20 bp, synthetic construct
  source 1..20: mol_type = other DNA
tggcaccaaagctgtatttg

SEQ ID NO: 511 - DNA, 20 bp, synthetic construct
  source 1..20: mol_type = other DNA
cacttcttacacttgcggac

SEQ ID NO: 512 - DNA, 20 bp, synthetic construct
  source 1..20: mol_type = other DNA
gcgtaatcccaaggatgtta

SEQ ID NO: 513 - DNA, 20 bp, synthetic construct
  source 1..20: mol_type = other DNA
agctgttttcacctctgttg

SEQ ID NO: 514 - DNA, 20 bp, synthetic construct
  source 1..20: mol_type = other DNA
tgacattccggcaagagacg

SEQ ID NO: 515 - DNA, 20 bp, synthetic construct
  source 1..20: mol_type = other DNA
ctgtgcaggtgatgttcatg

SEQ ID NO: 516 - DNA, 20 bp, synthetic construct
  source 1..20: mol_type = other DNA
cacactggatacagttgtct

SEQ ID NO: 517 - DNA, 20 bp, synthetic construct
  source 1..20: mol_type = other DNA
tgttgttttctcccatgact

SEQ ID NO: 518 - DNA, 20 bp, synthetic construct
  source 1..20: mol_type = other DNA
attcgttggacagccttcaa

SEQ ID NO: 519 - DNA, 20 bp, synthetic construct
  source 1..20: mol_type = other DNA
cgatggacgggatcttaggc

SEQ ID NO: 520 - DNA, 20 bp, synthetic construct
  source 1..20: mol_type = other DNA
cttatacaccgtgccgaacg

SEQ ID NO: 521 - DNA, 20 bp, synthetic construct
  source 1..20: mol_type = other DNA
atctgcacacaccagttgag

SEQ ID NO: 522 - DNA, 20 bp, synthetic construct
  source 1..20: mol_type = other DNA
gtgttttcaccagtacgttc

SEQ ID NO: 523 - DNA, 20 bp, synthetic construct
  source 1..20: mol_type = other DNA
tcatatggcttggatccaaa

SEQ ID NO: 524 - DNA, 20 bp, synthetic construct
  source 1..20: mol_type = other DNA
tcgtccatgtcttcttcatc

SEQ ID NO: 525 - DNA, 20 bp, synthetic construct
  source 1..20: mol_type = other DNA
aattgttgctggttgcactc

SEQ ID NO: 526 - DNA, 20 bp, synthetic construct
  source 1..20: mol_type = other DNA
ggaggaaggtgtcgtctatg

SEQ ID NO: 527 - DNA, 20 bp, synthetic construct
  source 1..20: mol_type = other DNA
gtgcctcactagctttaaag

SEQ ID NO: 528 - DNA, 20 bp, synthetic construct
  source 1..20: mol_type = other DNA
tataatcctggaaggcttgc

SEQ ID NO: 529 - DNA, 20 bp, synthetic construct
  source 1..20: mol_type = other DNA
agaaaaagtgtggcagcctc

SEQ ID NO: 530 - DNA, 20 bp, synthetic construct
  source 1..20: mol_type = other DNA
atccaaaaagccacggaacc

SEQ ID NO: 531 - DNA, 20 bp, synthetic construct
  source 1..20: mol_type = other DNA
aagacaactttccccttttg

SEQ ID NO: 532 - DNA, 20 bp, synthetic construct
  source 1..20: mol_type = other DNA
gctatcactgacgcatcaag

SEQ ID NO: 533 - DNA, 20 bp, synthetic construct
  source 1..20: mol_type = other DNA
ctccagattgtgtttgactg

SEQ ID NO: 534 - DNA, 20 bp, synthetic construct
  source 1..20: mol_type = other DNA
atgcatgctttctgaactgt

SEQ ID NO: 535 - DNA, 20 bp, synthetic construct
  source 1..20: mol_type = other DNA
gggtttaagtggtactcttc

SEQ ID NO: 536 - DNA, 20 bp, synthetic construct
  source 1..20: mol_type = other DNA
tggctttgaatgcatccaat

SEQ ID NO: 537 - DNA, 20 bp, synthetic construct
  source 1..20: mol_type = other DNA
cttcatcagcaactgaggtg

SEQ ID NO: 538 - DNA, 20 bp, synthetic construct
  source 1..20: mol_type = other DNA
tacaccagcattactagtca

SEQ ID NO: 539 - DNA, 20 bp, synthetic construct
  source 1..20: mol_type = other DNA
tgtaacatgccaaggctttg

SEQ ID NO: 540 - DNA, 20 bp, synthetic construct
  source 1..20: mol_type = other DNA
gtctactcacaaagcattcc

SEQ ID NO: 541 - DNA, 20 bp, synthetic construct
  source 1..20: mol_type = other DNA
aaaactgctttggcttggga

SEQ ID NO: 542 - DNA, 20 bp, synthetic construct
  source 1..20: mol_type = other DNA
cctgtagctgaagattgagg

SEQ ID NO: 543 - DNA, 20 bp, synthetic construct
  source 1..20: mol_type = other DNA
tactcagtgctgtgattcac

SEQ ID NO: 544 - DNA, 20 bp, synthetic construct
  source 1..20: mol_type = other DNA
attactgtgtgtttgttggc

SEQ ID NO: 545 - DNA, 20 bp, synthetic construct
  source 1..20: mol_type = other DNA
ttgctacaccaggaacatga

SEQ ID NO: 546 - DNA, 20 bp, synthetic construct
  source 1..20: mol_type = other DNA
gctttatgttctcatgttct

SEQ ID NO: 547 - DNA, 20 bp, synthetic construct
  source 1..20: mol_type = other DNA
gaccactgtagagtggatac

SEQ ID NO: 548 - DNA, 20 bp, synthetic construct
  source 1..20: mol_type = other DNA
aaccttgctaagtgacacgt

SEQ ID NO: 549 - DNA, 20 bp, synthetic construct
  source 1..20: mol_type = other DNA
ctcagatacttgtttccacg

SEQ ID NO: 550 - DNA, 18 bp, synthetic construct
  source 1..18: mol_type = other DNA
agacattctcgtcaagat

SEQ ID NO: 551 - DNA, 10 bp, synthetic construct
  source 1..10: mol_type = other DNA
ctgagtgttg

SEQ ID NO: 552 - DNA, 10 bp, synthetic construct
  source 1..10: mol_type = other DNA
aagtgactgt

SEQ ID NO: 553 - DNA, 10 bp, synthetic construct
  source 1..10: mol_type = other DNA
acatcatagt

SEQ ID NO: 554 - DNA, 20 bp, synthetic construct
  source 1..20: mol_type = other DNA
acagtcacttcaacactcag

(19) United States
(12) Patent Application Publication
(10) Pub. No.: US 2025/0179564 A1
(43) Pub. Date: Jun. 5, 2025

(54) Methods for Rapid, Scalable, Amplified Nucleic Acid Detection In Situ

(71) Applicant: THE TRUSTEES OF THE UNIVERSITY OF PENNSYLVANIA, Philadelphia, PA (US)

(72) Inventors: Arjun Raj, Philadelphia, PA (US); Ian Dardani, Philadelphia, PA (US); Benjamin Emert, Philadelphia, PA (US); Sara Rouhanifard, Philadelphia, PA (US)

(21) Appl. No.: 18/834,477

(22) PCT Filed: Feb. 7, 2023
(86) PCT No.: PCT/US2023/062143

§ 371 Date: Jul. 30, 2024

Related U.S. Application Data
(60) Provisional application No. 63/307,918, filed on Feb. 8, 2022.
(60) Provisional application No. 63/309,313, filed on Feb. 11, 2022.
(60) Provisional application No. 63/319,818, filed on Mar. 15, 2022.

(51) Int. Cl. C12Q 1/6841; C12Q 1/682

(52) U.S. Cl. CPC C12Q 1/6841; C12Q 1/682; C12Q 2600/166

(57) ABSTRACT

The present invention provides novel methods for exponential amplification of nucleic acid's fluorescence in situ hybridization (FISH) signal with high sensitivity and specificity. The present method thereby allows for FISH to be used in high-throughput screening methods and diagnostics. In one aspect, the invention comprises designing a primary click-amplifying FISH (clampFISH) probe for binding to a target sequence.

### CROSS-REFERENCE TO RELATED APPLICATIONS

The present application claims priority under 35 U.S.C. § 119 (e) to U.S. Provisional Patent Application No. 63/307,918, filed Feb. 8, 2022, U.S. Provisional Patent Application No. 63/309,313, filed Feb. 11, 2022, and U.S. Provisional Patent Application No. 63/319,818 filed Mar. 15, 2022, all of which are incorporated herein by reference in their entireties.

### STATEMENT REGARDING FEDERALLY SPONSORED RESEARCH OR DEVELOPMENT

This invention was made with government support under HL129998 and HG007743 awarded by the National Institutes of Health. The government has certain rights in the invention.

### SEQUENCE LISTING

The present application contains a Sequence Listing in XML format and is herein incorporated by reference in its entirety. Said XML file, created on Feb. 6, 2023, is named 046483_7357WO1_SequenceListing.xml and is 4,096 bytes in size.

### BACKGROUND OF THE INVENTION

Single molecule RNA FISH methods localize multiple fluorescent dye molecules to a target RNA, typically using complementary DNA probes that, in early designs, were directly labeled with fluorescent dyes. This labeling approach, however, produces only weak signal intensities that hinders its use in high-background tissue sections and also requires long imaging times. To amplify the signal, there are now multiple single molecule fluorescence in situ hybridization (smFISH) methods that build molecular scaffolds on the target RNA, providing a larger addressable sequence for fluorescent labeling. Each of these amplified methods, however, requires compromises in accuracy, multiplexing capacity, or cost. Thus, there remains a need in art for methods that permit accurate and flexible multiplexing and amplification of a nucleic acid signal with high sensitivity and specificity. The present invention addresses this unmet need.

### SUMMARY OF THE INVENTION

In one aspect, the invention provides a primary click-amplifying FISH (clampFISH) probe comprising:
a first oligonucleotide having
   (a) a target-specific oligonucleotide,
   wherein the target-specific oligonucleotide is about 30 nucleotides in length and comprises a continuous target-specific binding region;
   (b) a first flanking oligonucleotide,
   wherein the first flanking oligonucleotide is about 10 nucleotides in length,
   wherein the first flanking oligonucleotide is at the 5′ end of the target-specific oligonucleotide;
   (c) a second flanking oligonucleotide at the 3′ end of the target-specific sequence,
   wherein the second flanking oligonucleotide is about 10 nucleotides in length,
   wherein the second flanking oligonucleotide is at the 3′ end of the target-specific sequence; and
   wherein the 3′ end of the first oligonucleotide comprises an azide moiety;
a second oligonucleotide having
   (d) an amplifier-specific oligonucleotide,
   wherein the amplifier-specific oligonucleotide is about 30 nucleotides in length,
   (e) a first universal oligonucleotide,
   wherein the first universal oligonucleotide is about 18 nucleotides in length,
   wherein the first universal oligonucleotide is at the 5′ end of the amplifier-specific oligonucleotide,
   (f) a second universal oligonucleotide,
   wherein the second universal oligonucleotide is about 10 nucleotides in length,
   wherein the second universal oligonucleotide is at the 3′ end of the amplifier-specific oligonucleotide; and
   wherein the 5′ end of the second oligonucleotide comprises an alkyne moiety;
   wherein the 5′ end of the first oligonucleotide is ligated to the 3′ end of the second oligonucleotide, and.
wherein the 3′ end of the first oligonucleotide can be covalently locked to the 5′ end of the second oligonucleotide using click chemistry to circularize the primary clampFISH probe.

In certain embodiments, the first universal oligonucleotide is AGACATTCTCGTCAAGAT (SEQ ID NO: 550). In certain embodiments, the second universal oligonucleotide is CTGAGTGTTG (SEQ ID NO: 551).

In another aspect, the invention provides an amplifier probe comprising:
(a) a backbone comprising about 60 nucleotides, wherein the backbone is formed by concatenating two oligonucleotides (landing pad 1 and landing pad 2), wherein the landing pad 1 and the landing pad 2 each is about 30 nucleotides in length and comprises a sequence for binding to another amplifier probe;
(b) a first binding arm at the 3′ end of the landing pad 1, wherein the first binding arm is about 15 nucleotides in length;
(c) a second binding arm at the 5′ end of the landing pad 2, wherein the second binding arm is about 15 nucleotides in length;
wherein when the amplifier probe is a secondary amplifier probe, the first and the second binding arm together comprise a sequence that is reverse complementary to the landing pad 1 and/or the landing pad 2 of a tertiary amplifier probe or to an amplifier-specific oligonucleotide of a primary clampFISH probe;
wherein when the amplifier probe is the tertiary amplifier probe, the first and the second binding arm together comprise a sequence that is reverse complementary to the landing pad 1 and/or the landing pad 2 of the secondary amplifier probe,
wherein the 5′ end of the amplifier probe comprises as alkyne moiety and the 3′ end of the amplifier probe comprises an azide moiety,
wherein the 5′ end of the amplifier probe can be covalently locked to its 3′ end to circularize the amplifier probe.

In yet another aspect, the invention provides a method of exponentially amplifying the signal of a primary click-amplifying FISH (clampFISH) probe, the method comprising:
(a) hybridizing the primary clampFISH probe, described elsewhere herein, to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescence microscopy and/or flow cytometry.

In yet another aspect, the invention provides a method of detecting a target nucleic acid in a sample, the method comprising:
(a) hybridizing the primary clampFISH probe, described elsewhere herein, to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescent microscopy and/or flow cytometry.

In yet another aspect, the invention provides a kit comprising at set of primary click-amplifying FISH (clampFISH) probes as described elsewhere herein, a set of secondary amplifier probes, a set of tertiary amplifier probes, a set of amplifier-specific oligonucleotides, a set of dye-coupled DNA readout probes, a ligase, a hybridization solution, and a click chemistry agent for signal amplification and detection of nucleic acids in a sample and instructions for use thereof.

In yet another aspect, the invention provides a method of synthesizing a primary clampFISH probe by ligating a first oligonucleotide to a second oligonucleotide, wherein
the first oligonucleotide comprises:
(a) a target-specific oligonucleotide,
wherein the target-specific oligonucleotide is about 30 nucleotides in length and comprises a contiguous target-specific binding region;
(b) a first flanking oligonucleotide at the 5′ end of the target-specific oligonucleotide,
wherein the first flanking oligonucleotide comprises about 10 nucleotides;
(c) a second flanking oligonucleotide at the 3′ end of the target-specific sequence,
wherein the second flanking oligonucleotide comprises about 10 nucleotides; and
wherein the 3′ end of the first oligonucleotide comprises an azide moiety;
the second oligonucleotide comprises:
(d) an amplifier-specific oligonucleotide,
wherein the amplifier-specific oligonucleotide is about 30 nucleotides in length,
(e) a first universal oligonucleotide,
wherein the first universal oligonucleotide is about 18 nucleotides in length, and
wherein the first universal oligonucleotide is at the 5′ end of the amplifier-specific oligonucleotide,
(f) a second universal oligonucleotide,
wherein the second universal oligonucleotide is about 10 nucleotides in length, and
wherein the second universal oligonucleotide is at the 3′ end of the amplifier-specific sequence; and
wherein the 5′ end of the second oligonucleotide comprises an alkyne moiety;
wherein the 5′ end of the first oligonucleotide is ligated to the 3′ end of the second oligonucleotide, and.
wherein the 3′ end of the first oligonucleotide can be covalently locked to the 5′ end of the second oligonucleotide using click chemistry to circularize the primary clampFISH probe.

In certain embodiments, the azide moiety is N6-(6-Azido) hexyl-dATP. In certain embodiments, the azide moiety is added to the 3′ end of the primary clampFISH probe using terminal transferase enzyme.

In certain embodiments, the alkyne moiety is hexynyl.

In certain embodiments, the primary clampFISH probe is one selected from SEQ ID NO: 453 to SEQ ID NO: 467.

In certain embodiments, the GC content of each of the binding arms is about 45% to about 55%.

In certain embodiments, the alkyne moiety is hexynyl.

In certain embodiments, the amplifier probe is one selected from the SEQ ID NO: 423 to SEQ ID NO: 452.

In certain embodiments, the step (f) is repeated 1, 2, 3, 4, 5, 6, 7, 8, 9 or 10 times.

In certain embodiments, the length of the primary clampFISH probe is about 109 nucleotides.

In certain embodiments, the length of each of the secondary and the tertiary amplifier probes is about 90 nucleotides.

In certain embodiments, each of the secondary and the tertiary amplifier probes are as described elsewhere herein.

In certain embodiments, the set of secondary and tertiary amplifier probes comprises at least 2 probes.

In certain embodiments, the length of the readout probe is about 12 to about 20 nucleotides.

In certain embodiments, the readout probe can be removed from the amplifier probe.

In certain embodiments, the click chemistry agent catalyzes an azide-alkyne cycloaddition thereby circularizing the primary clampFISH probe and covalently locking the secondary and the tertiary amplifier probes around their respective nucleic acid target.

In certain embodiments, the click chemistry is catalyzed by copper (I), copper (II) or ruthenium.

In certain embodiments, the primary clampFISH probe, the secondary amplifier probes and the tertiary amplifier probes are DNA probes.

In certain embodiments, the primary clampFISH probe, the secondary amplifier probes and the tertiary amplifier probes are one selected from the group consisting of peptide nucleic acid (PNA), locked nucleic acid (LNA), and 2′-O-Methyl RNA.

In certain embodiments, the target nucleic acid is a DNA or an RNA.

In certain embodiments, the RNA is selected from the group consisting of messenger RNA, intronic RNA, exonic RNA, and non-coding RNA.

In certain embodiments, the tertiary amplifier probe is identical to the secondary amplifier probe.

In certain embodiments, the tertiary amplifier probe is not identical to the secondary amplifier probe.

In certain embodiments, the method allows simultaneous detection of multiple target nucleic acids in the sample.

In certain embodiments, the method allows detection of the target nucleic acid using a low magnification microscopy.

In certain embodiments, the primary clampFISH probe is one selected from SEQ ID NO: 453 to SEQ ID NO: 467.

In certain embodiments, the secondary amplifier probe is one selected from SEQ ID NO: 423 to SEQ ID NO: 437.

In certain embodiments, the tertiary amplifier probe is one selected from SEQ ID NO: 438 to SEQ ID NO: 452.

In certain embodiments, the readout probe is one selected from SEQ ID NO: 358 to SEQ ID NO: 392.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1E illustrate that clampFISH 2.0 enables fast, cost-effective, exponential amplification of multiplexed RNA signal in situ. (FIG. 1A) Schematic of clampFISH 2.0. (FIG. 1B) clampFISH 2.0 primary probes feature a design, where oligonucleotides modified for use with click chemistry can be re-used for all probes in any primary probe set, rather than being designed specifically for one region on a particular gene, thus greatly reducing the overall probe cost from that of clampFISH 1.0. This new probe design also permits higher-throughput synthesis by allowing all primary probes for a given gene to be made in a pool. (FIG. 1C) UBC clampFISH 2.0 at round 10 in WM989 A6-G3 cells, imaged with a 10× objective with the sizes of the smaller 20× and 60× fields of view overlaid. (FIG. 1D) UBC clampFISH 2.0 in WM989 A6-G3 cells shown at progressively higher rounds of amplification at 60×, 20× and 10× magnifications. (FIG. 1E) Left: UBC clampFISH 2.0 spots intensity (normalized to the median intensity from round 1) over progressively higher rounds of amplification, with the median intensity from rounds 2, 4, 6, 8 and 10 fit to an exponential curve. Right: spot intensities at rounds 2 and 8 when the copper catalyst is included or not included in the click reaction.

FIGS. 2A-2B illustrate that clampFISH 2.0 accurately quantifies RNA spot counts at low-powered magnification. (FIG. 2A) Top: schematic diagram of labeling of the same RNA with clampFISH 2.0 and conventional single-molecule RNA FISH, probing non-overlapping regions of the RNA. Middle: image of DDX58 clampFISH 2.0 spots with readout probes labeled in Alexa Fluor 594 and imaged at ×20 magnification. Bottom: image of conventional single-molecule RNA FISH (labeled with Cy3) targeting non-overlapping regions of DDX58 at ×60 magnification in the same cell. Scale bar, 5 μm. (FIG. 2B), Comparison of the spot counts between clampFISH 2.0 at ×20 magnification and conventional single-molecule RNA FISH at ×60 magnification. ClampFISH 2.0 was performed for 10 genes, the 10 scaffolds were amplified in parallel to round 8, then added a single pair of readout probes to label a scaffold corresponding to AXL (left; in drug-resistant WM989 A6-G3 RC4 cells), EGFR (middle; in drug-resistant WM989 A6-G3 RC4 cells), or DDX58 (right; in drug-naive WM989 A6-G3 cells). In two biological replicates spots were counted for clampFISH 2.0 at ×20 magnification and conventional single-molecule RNA FISH at ×60 magnification, which targeted non-overlapping regions of the same RNAs, as shown in FIG. 2A.

FIGS. 3A-3E illustrate that clampFISH 2.0 rapidly identifies rare cellular subpopulations in cell lines and tissue. (FIG. 3A) In the high-throughput profiling experiment, clampFISH 2.0 was performed for 10 genes in 1.3 million drug-naive WM989 A6-G3, with (FIG. 3B) images at 20× magnification of 3 example cells with 10 genes probed throughout 3 readout cycles. (FIG. 3C) 42,802 cells (5.9% of the 722,298 cells passing quality control checks) that expressed one or more of 8 cancer marker genes (WNT5A, DDX58, AXL, NGER, FN1, EGFR, ITGA3, MMP 1) were detected and hierarchical clustering was performed on this population. (FIG. 3D) A 20× magnification scan of DAPI in a fresh frozen xenograft tumor model with human WM989-A6-G3-Cas9-5a3 cells injected into a mouse. (FIG. 3E) 20× magnification images of clampFISH 2.0 spots in the same tissue section as in (FIG. 3D), probing for the same 10 genes as in (FIG. 3B).

FIG. 4 shows that clampFISH 2.0 amplifies GFP mRNA signal. GFP clampFISH 2.0 spots in drug-naive H2B-GFP WM989 A6-G3 cells (top) and vemurafenib-resistant WM989 A6-G3 RC4 cells (bottom) with a 20 nucleotide secondary-targeting readout probe (labeled with Atto 647N) and conventional single-molecule RNA FISH probes (labeled with Alexa 555) targeting different regions of the same RNA. As expected, bright GFP clampFISH 2.0 spot counts were observed in cells with nuclear-localized GFP signal observed, but not in cells without the H2B-GFP construct.

FIG. 5 shows that clampFISH 2.0 amplifies EGFR mRNA signal. EGFR clampFISH 2.0 spots in drug-naive H2B-GFP WM989 A6-G3 cells (top) and vemurafenib-resistant WM989 A6-G3 RC4 cells (bottom) with a 20 nucleotide secondary-targeting readout probe (labeled with Atto 647N) and conventional single-molecule RNA FISH probes (labeled with Cy3) targeting different regions of the same RNA. As expected from bulk RNA-sequencing data, many more EGFR clampFISH 2.0 spots were observed in vemurafenib-resistant cells than in the drug-naive cells.

FIGS. 6A-6B show that clampFISH 2.0 amplifies signal exponentially. (FIG. 6A) In an amplification characterization experiment, clampFISH 2.0 was performed with amplification to varying rounds (round 1, 2, 4, 6, 8, and 10) and then four readout probes were hybridized to measure the spot intensities, with the median intensity from rounds 2, 4, 6, 8 and 10 fit to an exponential curve (labeled values are median intensities). It was found that every round the spot intensities grew by a factor of 1.457, 1.586, 1.406, and 1.527 for each probe set respectively. With a hypothetical 2:1 binding ratio of each amplifier probe to the previous probe, these factors suggest a per-probe binding efficiency of 73%, 79%, 70%, and 76%, respectively. (FIG. 6B) Replicate 2 of the same experiment as in (FIG. 6A), where the spot intensities grew by a factor of 1.525, 1.678, 1.496, and 1.628, suggesting per-probe binding efficiencies of 76%, 84%, 75%, and 81%, respectively. For spot counts associated with each condition in FIGS. 6A and 6B. Circles are median values and bounds of boxes are 25th and 75th percentiles.

FIGS. 7A-7B show that clampFISH 2.0 signal amplification is dependent on the click reaction. (FIG. 7A) In an amplification characterization experiment, the clampFISH 2.0 amplification steps to rounds 2 and 8, both with and without the copper sulfate catalyst included in the click reaction (labeled values are median intensities) were performed. (FIG. 7B) a biological replicate (different passage) of the same experiment as in (FIG. 7A a). No amplification was observed from round 2 to round 8 in the absence of the copper catalyst, confirming that the click reaction is an essential step for clampFISH 2.0. For spot counts associated with each condition in FIGS. 7A and 7B. Circles are median values and bounds of boxes are 25th and 75th percentiles.

FIG. 8 show results of a screen of amplifier probe sets that revealed designs with a high level of signal amplification. In a screen for amplifier probe sequences, 15 GFP (left) and 15 EGFR (right) primary probe sets ligated were hybridized to one of 15 corresponding amplifier probe set-specific oligonucleotides, then amplified each to round 8. The clampFISH 2.0 scaffolds were labeled with 20 nucleotide secondary-targeting readout probes (coupled to Atto 647N) and performed conventional single-molecule RNA FISH (GFP probes in Alexa 555, EGFR probes in Cy3) to non-overlapping regions of the same mRNA as the primary probes. The number of conventional single-molecule RNA FISH spots were counted the in each segmented cell, equivalent number of the highest-intensity clampFISH 2.0 spots were taken from that cell, and these clampFISH 2.0 spot intensities (11,252 GFP and 881 EGFR outliers, out of 294,220 and 22,861 total points respectively, are not shown) were plotted.

FIG. 9 illustrates that amplifier probe sets can modularly be used with various primary probe sets. The median spot intensity generated by each clampFISH 2.0 amplifier set from the amplifier screen experiment when used with primaries for GFP (x-axis) or EGFR (y-axis) were plotted. A strong correlation was observed between the two primary probe sets, suggesting that gene-specific effects on amplification play a minimal role in their performance. The slope of the regression suggests a nearly 2-fold increase in spot intensities when amplifier sets were used with the EGFR probe set over the GFP probe set, likely as a result of the 3-fold higher number of primary probes (30 for EGFR vs. 10 for GFP).

FIG. 10 illustrates that amplifier probe sets amplify signal similarly when used alone vs. when used in a pool of 10 amplifier probes. 10 GFP-targeting primary probe sets were hybridized, each ligated to a different amplifier-binding oligonucleotide, and amplified each in one of two ways: with its corresponding amplifier probe set alone or with a pool of all 10 amplifier sets. Plotted are the intensities of the 10,000 highest-intensity spots from 40 segmented cells per condition (379 ‘alone’ spots outliers and 418 ‘pooled’ spot outliers not shown).

FIGS. 11A-11B illustrate that clampFISH 2.0 readout probe signal can be removed with a high-stringency wash. (FIG. 11A) Boxplots of clampFISH 2.0 spots per cell detected above a chosen gene-specific threshold for 10 genes before and after the readout probe stripping protocol. Shown for each gene are spot counts from one of two melanoma lines with higher expression for that gene (for NGFR: drug-naive WM989 A6-G3 cells; for all genes: vemurafenib-resistant WM989 A6-G3 RC4 cells). Each condition contains 39-48 segmented cells where each cell is represented in both the before-stripping and the after-stripping data. The box and whiskers for the after-strip data are at 0 spots and thus are not visible, except for FN1 which has an interquartile range from 0 to 2.5 spots and a whisker extending to 6 spots. (FIG. 11B) Depicting the same data as in (FIG. 11A) for only data below 500 spots per cell.

FIGS. 12A-12B illustrate that signal from the previous readout cycle is removed after a high-formamide strip. (FIG. 12A) Example images of clampFISH 2.0 spots at 20× magnification before the readout probe hybridization (top row), after adding readout probes (middle row), and after stripping off readout probes (bottom row). The first three columns are from readout cycle 1, the next three are from readout cycle 2, and the last 4 columns are from readout cycle 3. Each column's images are from the same channel (with the corresponding readout probe dye indicated), exposure time (as indicated in milliseconds), and are contrasted identically. (FIG. 12B) Example images as in (FIG. 12A) at a different position on the plate.

FIG. 13 illustrates that clampFISH 2.0 scaffolds remain stably bound after multiple rounds of readout stripping and storage at 4° C. for 4 months. Images of clampFISH 2.0 spots from a 20× objective over readout cycles where 4 sets of readout probes were repeatedly used which label (from top to bottom) AXL, WNT5A, DDX58, and UBC clampFISH 2.0 scaffolds. Column 1: readout cycle 1. Column 2: readout cycle 1, re-imaged after removing the sample from the microscope stage and stored overnight at 4° C. Column 3: after stripping off readout probes from readout cycle 1. Column 4: readout cycle 4, where readout cycle 1 was repeated after readout cycles 2 and 3 (where different sets of genes were labeled). Column 5: readout cycle 5, performed after storing the sample at 4° C. in 2×SSC for 4 months. DAPI overlay is contrasted separately for each column. Each row of readout cycle 5 (column 5) is contrasted with 180% the intensity range of the first four columns. The cycle 5 signal presumably appeared brighter due to changes in the microscope's optics during that time frame (e.g. greater sample illumination or increased transmission to the sensor).

FIG. 14A illustrates that clampFISH 2.0 scaffolds remain stably bound after multiple rounds of readout stripping and storage at 4° C. for 4 months. clampFISH 2.0 spots per cell for (from top to bottom) WNT5A, DIX58, and AX1, from readout cycle 1 (x-axis) plotted against 3 additional rounds of imaging for the same probed scaffold: re-imaged readout cycle 1 (column 1 plots); readout cycle 4, where the same readout probes were used as cycle 1 (column 2 plots); and readout cycle 5, where again we used the same readout probes as cycle 1 after being stored for 4 months in 4° C. (column 3 plots). Each spot is one of 44,227 cells. See FIG. 13 for experiment workflow schematic.

FIG. 14B illustrates that clampFISH 2.0 scaffolds remain stably bound after multiple rounds of readout stripping (replicate 2). Technical replicate 2 of the experiment from FIG. 14A, but without readout cycle 5, clampFISH 2.0 spots per cell for (from top to bottom) WNT5A, DDX58, and AXL from readout cycle 1 (x-axis) plotted against 2 additional rounds of imaging for the same probed scaffold: re-imaged readout cycle 1 (column 1 plots); and readout cycle 4, where the same readout probes were used as cycle 1 (column 2 plots). Each spot is one of 89,545 cells. See FIG. 13 for experiment workflow schematic.

FIG. 15 shows clampFISH 2.0 in formalin-fixed paraffin embedded (FFPE) tissue. ClampFISH 2.0 was performed for ten genes in FFPE tumor tissue derived from human WM4505-1 cells injected into a mouse. The readout probes for four clampFISH 2.0 scaffolds were then hybridized, from left to right: UBC (Atto 488), NGFR (Cy3), MMP1 (Alexa Fluor 594), and AXL (Atto 647N). Shown are images that were taken at 20× magnification.

FIG. 16 shows that clampFISH 2.0 eliminates the bright, non-specific fluorescent spots that were observed in clampFISH 1.0. Top left: clampFISH 1.0 targeting GFP in WM983b-GFP melanoma cells, amplified to round 6 with amplifier probes containing an internal Cy5 dye and imaged at 20× with a 3 second exposure time using a cooled CCD camera with a 13 μm pixel size. The two arrows point to two of the non-specific spots. Top right: clampFISH 2.0 targeting GFP in a mixed population of cells (a majority of WM989 A6-G3 H2B-GFP cells and fewer WM989 A6-G3 RC4 cells), amplified to round 8 with readout probes labeled with Atto 647N and imaged at 20× with a 1 second exposure time using a sCMOS camera with a 6.5 μm pixel size. Image shown is from the present work's ‘pooled amplifier experiment’, which was performed once. For all experiments performed in this work, similar results to those depicted here were observed. Bottom: zoomed-in views of the top images. The bright non-specific spots could be eliminated by introducing a number of centrifugation steps to both the primary probe and amplifier probe synthesis protocols. To perform this step, the solution was centrifuged in 1.5 mL tubes at 17,000 g for 20 minutes and transferred the top portion of the solution to a new tube and discarded the bottom portion. This step was performed twice after the enzymatic steps are complete, and once after ethanol precipitation (see FIGS. 21A-21B). Additionally, it was found that by adding the centrifugation step to completed clampFISH 1.0 probe solutions, the non-specific spots seen in that method could be reduced.

FIGS. 17A-17B show that clampFISH 2.0 spot sizes remain similar throughout the rounds of amplification. (FIG. 17A) Cropped images of spots from UBC clampFISH 2.0 with readout probes in Atto 488 at varying levels of amplification (from left to right: round 1, 2, 4, 6, 8, and 10) imaged with a 100×/1.45NA objective (65 nm pixel sizes). A spot with a representative (median) fitted amplitude was chosen for display. The minimum intensity and maximum intensity used for contrasting are shown below the images. Contrasting is applied equally to all images (top row) or set to each image's minimum and maximum values (bottom row). (FIG. 17B) ClampFISH 2.0 was performed to varying rounds of amplification using primary probes targeting UBC mRNA, amplifier set 9, and readout probes labeled in Atto 488 (top panels) or using primary probes targeting MITF, amplifier set 12, and readout probes labeled in Atto 647N (bottom panels). Samples were imaged with a 100×/1.45NA objective (65 nm pixel sizes) and each called spot was fit at its maximal-intensity z-plane to a 2D Gaussian distribution. Shown are the standard deviation of each spot's Gaussian fit (left panels), amplitude of each spot's Gaussian fit normalized to the round 1 median amplitude (middle panels), and each segmented cell's spot count (right panels). For the left and middle panels, circles and numbers shown are median values and bounds of boxes are 25 th and 75 th percentiles. For UBC data, n=923, 1437, 1968, 1737, 2251, 846 spots and for MITF data n=1206, 1219, 994, 1634, 1450, and 930 for rounds 1,2,4,6,8, and 10, respectively. For the right panel, circles are median values, bounds of boxes are 25 th and 75th percentiles, and whiskers extend to non-outlier minima and maxima, where data falling more than 1.5 times the interquartile range beyond the box bounds are considered outliers. Theoretical standard deviations of Gaussian approximations of diffraction-limited spots (0.212/NA; with paraxial optics assumptions) with wavelengths at the midpoints of the emission filters (535 nm for Cy3; 667 nm for Atto 647N) are 77.5 nm (Cy3) and 96.6 nm (Atto 647N).

FIG. 18 shows that the clampFISH 2.0 spot sizes are similar to conventional single-molecule RNA FISH spot sizes. Conventional single-molecule RNA FISH (smFISH) spot sizes are compared to clampFISH 2.0 spots imaged on the same day and to clampFISH 2.0 spots in a previous experiment. The samples were imaged with a 100×/1.45NA objective at 1×1 camera binning (65 nm pixel size) and fit the pixel values in the neighborhood of each spot to a 2D Gaussian distribution. Left: standard deviation of Gaussian-fitted spots for UBC smFISH labeled in Atto 488 and UBC clampFISH 2.0 amplified to round 1 or round 4 with readout probes labeled in Atto 488. Right: standard deviation of Gaussian-fitted spots for TOP2A smFISH labeled in Atto 647N and MITF clampFISH 2.0 amplified to round 1 or round 4 with readout probes labeled in Atto 647N. Values shown are the median standard deviations. For the left and right panels, circles and numbers shown are median values and bounds of boxes are 25th and 75th percentiles. For Atto 488 data (left panel), n=1053, 923, and 1968 (from left to right) and for Atto 647N data (right panel) n=1875, 1230, 2254, 1206, and 994 (from left to right).

FIG. 19 shows the clampFISH 2.0 quantifies RNA spot counts at 10× magnification. Depicting the same data as in FIG. 2B, but with clampFISH 2.0 spots imaged at 10× magnification. clampFISH 2.0 was performed for 10 genes, the 10 scaffolds were amplified in parallel to round 8, then a single pair of readout probes was added to label a scaffold corresponding to AXL (left; in drug-resistant WM989 A6-G3 RC4 cells), EGFR (middle; in drug-resistant WM989 A6-G3 RC4 cells), or DDX58 (right; in drug-naïve WM989 A6-G3 cells). In two biological replicates (top: replicate 1; bottom: replicate 2), spots were counted for clampFISH 2.0 at 10× magnification (y-axis) and conventional single-molecule RNA FISH at 60× magnification (x-axis), which targeted non-overlapping regions of the same RNAs. In replicate 2, imaging at 10× of DDX58 spots before conventional single-molecule RNA FISH was not performed.

FIGS. 20A-20B show that clampFISH 2.0 detects RNAs in presumptive human cells in tissue. clampFISH 2.0 was performed in a 6 μm fresh frozen tissue section of a dissected tumor, derived from human WM989-A6-G3-Cas9-5a3 cells injected into a mouse and fed chow containing the BRAFV600E inhibitor PLX4720. Shown are stitched maximum intensity projections of 20× image stacks with 5 z-planes at 1.2 μm z-step increments. (FIG. 20A) outlines around regions containing mostly presumptive human cells, demarcated based on nuclear morphology, showing DAPI staining alone (left) and DAPI with UBC clampFISH 2.0 signal overlaid (right), where images are from readout cycle 2. (FIG. 20B) clampFISH 2.0 scaffolds for 10 genes were probed across readout cycles 1 (left), 2 (middle), and 3 (right), where the UBC scaffold was probed each round as a positive control. The dyes on each readout probe set were (top to bottom): Atto488, Cy3, Alexa Fluor 594, and Atto647N. The experiment was performed twice with similar results.

FIGS. 21A and 21B both show a schematic of clampFISH 1.0 (top) and clampFISH 2.0 (bottom) probe synthesis protocols for primary probes (FIG. 21A) and amplifier probes (FIG. 21B).

FIG. 22 illustrates that clampFISH 2.0 spots colocalize with conventional single-molecule RNA FISH (smFISH) spots when probing the same RNA. ClampFISH 2.0 (10 primary probes; readouts in Atto 647N) and conventional smFISH probes (15 probes; labeled in Alexa 555) were both designed to target non-overlapping regions of GFP mRNA. ClampFISH 2.0 primaries were used with one of 15 amplifier sets (plots 1-15). To call conventional single-molecule RNA FISH spots cell-specific manual intensity thresholds were chosen, whereas to call clampFISH 2.0 spots a single threshold which was varied (x-axis) was chosen. Shown are the fraction of clampFISH 2.0 spots co-localizing with conventional smFISH and the fraction of conventional smFISH spots co-localizing with clampFISH 2.0 spots. Asterisks (*) mark the 10 amplifier sets that were used in later multiplexing experiments.

FIG. 23 illustrates that one-pot amplification protocol did not produce amplified spots. It was tested whether a one-pot amplification protocol, where the secondary probes, tertiary probes, and click reagents were added simultaneously, could produce amplified spots. In one-pot amplification #1, 10% formamide and 10% dextran sulfate were included in the one-pot mixture, whereas one-pot amplification #2 did not have these reagents. Also, clampFISH 2.0 was performed in the standard manner to rounds 1 (i.e. only primary probes) and round 4. Left: Normalized amplitudes of the MITF clampFISH 2.0 spots, where circles and numbers shown are median values and bounds of boxes are 25th and 75th percentiles. n=1230, 2254, 1743, and 1239 spots (from left to right). Right: MITF clampFISH 2.0 spot count per cell from 12 segmented cells per condition. Circles are median values, bounds of boxes are 25th and 75th percentiles, and whiskers extend to non-outlier minima and maxima, where data falling more than 1.5 times the interquartile range beyond the box bounds are considered outliers. The experiment was performed once. The spot intensities of both one-pot conditions were not higher than the intensities produced by the primaries alone (round 1), indicating that the tested one-pot amplification conditions are not a viable alternative to the standard step-wise amplification protocol. The two other primary probes and amplifier sets tested (FN1 with amplifier set 5, NGFR with amplifier set 1) also did not amplify in the one-pot conditions (data not shown).

FIG. 24 illustrates that clampFISH 2.0 detects transcription sites. Conventional single-molecule RNA FISH (smFISH) (top row) and clampFISH 2.0 (bottom row) probing non-overlapping regions of the same RNAs (from left to right: AXL, EGFR, and DDX58). Images of smFISH, labeled in Cy3, are maximum intensity projections of 5 z-planes at 0.5 μm z-steps taken with a 60× objective with 2 second exposure times for all RNAs. Images of clampFISH 2.0 are from a single plane taken with a 20× objective with exposure times of 1 second (AXL, DDX58) or 500 milliseconds (EGFR), with readout probes labeled in Atto 647N (AXL, EGFR) and Alexa Fluor 594 (DDX58). Arrows point to putative transcription sites with multiple RNA copies, identified as such from their nuclear localization and their increased spot intensity relative to other nuclear and cytoplasmic RNA spots. The experiment was performed twice with similar results.

FIG. 25 illustrates that off-target spots seen in clampFISH 1.0 precluded the identification of transcription sites. Maximum intensity projections of 20 z-planes from 100× magnification image stacks of clampFISH 1.0 targeting GFP mRNA in cells without GFP (WM983b cells, left) and in cells expressing GFP (WM983b-GFP cells, right) . . . . Data are typical for clampFISH 1.0 results, although the number of off-target spots can vary from experiment to experiment.

FIGS. 26A-26B show that mean clampFISH 2.0 spot counts are correlated with bulk RNA sequencing data. (FIG. 26A) Mean clampFISH 2.0 spot counts from 722,298 drug-naive WM989 A6-G3 cells (left) and 2,155 vemurafenib-resistant WM989 A6-G3 RC4 cells (right) for the 10 genes from the high-throughput profiling experiment (x-axis) and bulk RNA-seq transcripts per million (y-axis) for each of the two cell lines. (FIG. 26B) A technical replicate of the same experiment as in (FIG. 26A), but with data from 234,410 drug-naive WM989 A6-G3 cells and 5,150 vemurafenib-resistant WM989 A6-G3 RC4 cells, using the same bulk RNA sequencing data. It was observed that FN1 and MMP1, both of which have a lower mean clampFISH 2.0 spot count than would be expected from the remaining genes' trend, are expressed at particularly high levels in a subset of cells (see FIG. 3B), suggesting that optical crowding at 20× magnification may contribute to their under-counting by clampFISH 2.0.

FIG. 27 shows that mean fluorescent signal is not well-correlated with spot count density [without pre-readout signal subtraction] and reveals some saturation in spot counts due to optical crowding in rare cells with very high expression levels. Scatter plots of spot count per area of cellular segmentation versus mean background-subtracted fluorescent intensity in the cellular segmentation for 10 different genes probed across readout cycle 1 (left column), readout cycle 2 (middle column), and readout cycle 3 (right column), where scaffolds targeting UBC mRNA were probed on every cycle as a positive control. Each dot represents a cell. See Methods section for details.

FIG. 28 shows that mean fluorescent signal is not well-correlated with spot count density [with pre-readout signal subtraction] and reveals some saturation in spot counts due to optical crowding in rare cells with very high expression levels. Scatter plots, as depicted in FIG. 27, but with the mean signal before the addition of a given cycle's readout probes subtracted to correct for background from autofluorescence and residual readout probes from previous readout cycles. Data for readout cycle 1 is not available. See Methods section for details.

FIG. 29 shows that clustering of cells expressing one or more drug resistance markers. Technical replicate 2 of the high-throughput profiling experiment from FIG. 3C. clampFISH 2.0 was performed for 10 genes in 253,662 drug-naive WM989 A6-G3 cells. 24,685 cells (10.5% of the 234,410 cells passing quality control checks) that had high levels of one or more of 8 cancer marker genes (WNT5A, DDX58, AXL, NGFR, FN1, EGFR, ITGA3, MMP1) were detected and hierarchical clustering was performed on this population.

FIG. 30 illustrates that spot intensities of clampFISH 2.0 in a fresh frozen tissue sections were comparable to those in a cell line, while those from a formalin-fixed paraffin embedded (FFPE) tissue sections were dimmer. Gaussian-fitted spot amplitudes of clampFISH 2.0 targeting ITGA3 to round 8 in a cell line (human WM989 A6-G3 cells; left), an FFPE tissue section (human WM4505-1 cells implanted into a mouse; middle), and a fresh frozen tissue section (human WM989-A6-G3-Cas9-5a3 cells injected into a mouse), normalized to the median amplitude of the cell line spots. The clampFISH 2.0 primary and amplification steps were performed in parallel for all sample types, which were all imaged on the same microscope at 20× magnification with equivalent (1 second) exposure times. Numbers shown are median normalized amplitudes. Circles and numbers shown are median values and bounds of boxes are 25th and 75th percentiles. n=3234, 2973, and 1669 spots (from left to right).

### DETAILED DESCRIPTION

The present invention provides novel methods for exponential amplification of nucleic acids' fluorescence in situ hybridization (FISH) signal with high sensitivity and specificity. The present method thereby allows for FISH to be used in high-throughput screening methods and diagnostics.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of +20% or +10%, +5%, +1%, or +0.1% from the specified value, as such variations are appropriate. As used herein, the terms “alkyne group”, “alkyne moiety”, “alkyne” or “alkynyl” are used herein interchangeably. These terms employed alone or in combination with other terms, mean, unless otherwise stated, a stable straight, branched, or cyclic chain hydrocarbon group with a triple carbon-carbon bond, having the stated number of carbon atoms. Non-limiting examples include ethynyl and propynyl, and the higher homologs and isomers. Exemplary alkyl groups of use in the present invention contain between about one and about twenty-five carbon atoms (e.g. methyl, ethyl and the like). Straight, branched or cyclic hydrocarbon chains having eight or fewer carbon atoms will also be referred to herein as “lower alkyl” (e.g. cyclooctyne). In addition, the term “alkyl” as used herein further includes one or more substitutions at one or more carbon atoms of the hydrocarbon chain fragment.

The term “click chemistry,” as used herein, refers to the Huisgen cycloaddition or the 2,3-dipolar cycloaddition between an azide and a terminal alkyne to form a 1,2,4-triazole. Such chemical reactions can use, but are not limited to, simple heteroatomic organic reactants and are reliable, selective, stereospecific, and exothermic. As used herein, click chemistry also refers to a strain promoted azide alkyne cycloaddition (SpAAC) where a cyclooctyne is able to undergo azide-alkyne Huisgen cycloaddition under mild, physiological conditions in the absence of a copper (I) catalyst.

The term “mutation” as used herein refers to any change of one or more nucleotides in a nucleotide sequence.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology.

As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene. A “coding region” of an mRNA molecule also consists of the nucleotide residues of the mRNA molecule which are matched with an anti-codon region of a transfer RNA molecule during translation of the mRNA molecule or which encode a stop codon.

The coding region may thus include nucleotide residues corresponding to amino acid residues which are not present in the mature protein encoded by the mRNA molecule (e.g., amino acid residues in a protein export signal sequence).

As used herein, the term “covalently locks” refers to the interaction formed between clampFISH probes and the one or more regions of the target nucleic acid or between the various clampFISH probes, in each case as shown in the figures. Covalent locking does not require a covalent bond between the molecules.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids, which have been substantially purified from other components, which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

The term “fluorophore” as used herein refers to a composition that is inherently fluorescent or demonstrates a change in fluorescence upon binding to a biological compound or metal ion, i.e., fluorogenic. Fluorophores may contain substituents that alter the solubility, spectral properties or physical properties of the fluorophore. Numerous fluorophores are known to those skilled in the art and include, but are not limited to coumarin, cyanine, benzofuran, a quinoline, a quinazolinone, an indole, a benzazole, a borapolyazaindacene and xanthenes including fluorescein, rhodamine and rhodol as well as other fluorophores known in the art.

A “portion” of a polynucleotide means at least at least about five to about fifty sequential nucleotide residues of the polynucleotide. It is understood that a portion of a polynucleotide may include every nucleotide residue of the polynucleotide.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label,” as used herein, refers to a chemical moiety or protein that is directly or indirectly detectable (e.g., due to its spectral properties, conformation or activity) when attached to a target or compound and used in the present methods, including reporter molecules and carrier molecules. The label can be directly detectable (fluorophore) or indirectly detectable (hapten or enzyme). Such labels include, but are not limited to, radiolabels that can be measured with radiation-counting devices; pigments, dyes or other chromogens that can be visually observed or measured with a spectrophotometer; spin labels that can be measured with a spin label analyzer; and fluorescent labels (fluorophores), where the output signal is generated by the excitation of a suitable molecular adduct and that can be visualized by excitation with light that is absorbed by the dye or can be measured with standard fluorometers or imaging systems, for example. The label can be a chemiluminescent substance, where the output signal is generated by chemical modification of the signal compound; a metal-containing substance; or an enzyme, where there occurs an enzyme-dependent secondary generation of signal, such as the formation of a colored product from a colorless substrate. The term label can also refer to a “tag” or hapten that can bind selectively to a conjugated molecule such that the conjugated molecule, when added subsequently along with a substrate, is used to generate a detectable signal. For example, one can use biotin as a tag and then use an avidin or streptavidin conjugate of horseradish peroxidate (HRP) to bind to the tag, and then use a calorimetric substrate (e.g., tetramethylbenzidine (TMB)) or a fluorogenic substrate such as Amplex Red reagent (Molecular Probes, Inc.) to detect the presence of HRP. Numerous labels are known by those of skill in the art and include, but are not limited to, particles, fluorophores, haptens, enzymes and their calorimetric, fluorogenic and chemiluminescent substrates and other labels known in the art.

“Naturally occurring” as used herein describes a composition that can be found in nature as distinct from being artificially produced. For example, a nucleotide sequence present in an organism, which can be isolated from a source in nature, and which has not been intentionally modified by a person in the laboratory, is naturally occurring.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

The terms “patient,” “subject,” “individual,” and the like are used interchangeably herein, and refer to any animal, or cells thereof whether in vitro or in situ, amenable to the methods described herein. Preferably, the patient, subject or individual is a mammal, and more preferable, a human.

“Variant” as the term is used herein, is a nucleic acid sequence or a peptide sequence that differs in sequence from a reference nucleic acid sequence or peptide sequence respectively, but retains essential properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

RNA labeling in situ has enormous potential to reveal transcript levels in its natural context, but it remains challenging to produce high levels of signal while also enabling multiplexed detection of multiple RNA species simultaneously. Described here is a method, clampFISH 2.0, that uses an exponential inverted padlock design to efficiently amplify and detect signal from many RNA species at once, also reducing time and cost compared to clampFISH 1.0. The increased throughput afforded by multiplexed signal amplification and sequential detection is leveraged by demonstrating the ability to detect 10 different RNA species in over 1 million cells. It is also shown that clampFISH 2.0 works in tissue sections.

### Probes

Primary clampFISH Probe

In one aspect, the invention provides a primary clampFISH probe. In certain embodiments the primary clampFISH probe comprises a first oligonucleotide.

In certain embodiments, the first oligonucleotide includes a target-specific oligonucleotide. In certain embodiments, the target-specific oligonucleotide is about 30 nucleotides in length and comprises a continuous target-specific binding region.

In certain embodiments, the target specific oligonucleotide is flanked by a first flanking oligonucleotide at its 5′ end. In certain embodiments, the first flanking oligonucleotide is about 10 nucleotides in length.

In certain embodiments, the target specific oligonucleotide is flanked by a second flanking oligonucleotide at its 3′ end. In certain embodiments, the second flanking oligonucleotide is about 10 nucleotides in length.

In certain embodiments the primary clampFISH probe comprises a second oligonucleotide.

In certain embodiments, the second oligonucleotide includes an amplifier-specific oligonucleotide. In certain embodiments, the amplifier-specific oligonucleotide is about 30 nucleotides in length.

In certain embodiments, the second oligonucleotide includes a first universal oligonucleotide. In certain embodiments, the first universal oligonucleotide flanks the 5′ end of the amplifier-specific oligonucleotide. In certain embodiments the first universal oligonucleotide is about 18 nucleotides in length. In certain embodiments, the first universal oligo nucleotide comprises a GC-content of about 35% to about 65% to avoid formation of secondary structures. In certain embodiments, the first universal oligo nucleotide comprises a GC-content of about 35%, 40%, 45%, 50%, 55%, 60%, or about 65%. In certain embodiments, the first universal oligonucleotide is AGACATTCTCGTCAAGAT (SEQ ID NO:550).

In certain embodiments, the second oligonucleotide includes a second universal oligonucleotide. In certain embodiments, the second universal oligonucleotide flanks the 3′ end of the amplifier-specific oligonucleotide. In certain embodiments, the second universal oligonucleotide is about 10 nucleotides in length. In certain embodiments, the second universal oligonucleotide comprises GC-content such that formation of secondary structure is avoided. In certain embodiments, the second nucleotide is CTGAGTGTTG (SEQ ID NO: 551).

In certain embodiments, the 5′ end of the first oligonucleotide is ligated to the 3′ end of the second oligonucleotide to form primary clampFISH probes having a total length of about 109 nucleotides.

In certain embodiments, the 3′ end of the first oligonucleotide comprises an azide moiety. In certain embodiments, the azide moiety is added to the 3′ end using terminal transferase enzyme. In certain embodiments the azide moiety is an N6-(6-Azido) hexyl-dATP.

In certain embodiments, the 5′ end of the second oligonucleotide comprises an alkyne moiety. In certain embodiments, the alkyne moiety is hexynyl.

In certain embodiments, the 3′ end of the first oligonucleotide is covalently locked to the 5′ end of the second oligonucleotide using click chemistry to form a circularized clampFISH probe.

In certain embodiments, the first oligonucleotide and the second oligonucleotide do not comprise azide and alkyne modifications. In certain embodiments, circularization of the clampFISH probe is facilitated by a ligase, such as a DNA ligase. In certain embodiments, the first oligonucleotide and the second oligonucleotide are modified to comprise biotin and streptavidin, respectively, (or vice versa), and circularization of the primary clampFISH probe is facilitated via biotin-streptavidin interactions.

In certain embodiments, the primary clampFISH probe is one selected from SEQ ID NO: 453 to SEQ ID NO: 467.

### Amplifier Probes

In certain embodiments, the invention provides an amplifier probe. In certain embodiments, the amplifier probe is about 90 nucleotides in length. In certain embodiments, the amplifier probe is a secondary amplifier probe or a tertiary amplifier probe. In certain embodiments, the tertiary amplifier probe has the same sequence as that of the secondary amplifier probe. In certain embodiments, the tertiary amplifier probe has a different sequence from that of the secondary amplifier probe.

In certain embodiments, the amplifier probe comprises a backbone that is about 60 nucleotides in length. In certain embodiments, the backbone is formed by concatenating two oligonucleotides (landing pad 1 and landing pad 2), each of which comprise about 30 nucleotides long “landing pad” sequence for binding to another amplifier probe. In certain embodiments, each of the 30-nucleotides long landing pad comprises about 50% GC-content. In certain embodiments, the 30-nucleotides long landing pad is designed to contain bases AT at its center. In certain embodiments, optionally, a spacer sequence is included between the two landing pads (landing pad 1 and landing pad 2).

In certain embodiments, the amplifier probe further comprises a first binding arm at the 3′ end of the backbone, wherein the first binding arm is about 15 nucleotides in length. In certain embodiments, the first binding arm has a GC-content of about 45% and to about 55%. In certain embodiments, the first binding arm has a GC content of about 45% 46%, 47%, 48%, 49%, 50%, 51%, 52%, 53%, 54%, or about 55%.

In certain embodiments, the amplifier probe further comprises a second binding arm at the 5′ end of the backbone, wherein the second binding arm is about 15 nucleotides in length. In certain embodiments, the second binding arm has a GC-content of about 45% to about 55%. In certain embodiments, the second binding arm has a GC content of about 45% 46%, 47%, 48%, 49%, 50%, 51%, 52%, 53%, 54%, or about 55%.

In certain embodiments, optionally, spacer sequences are included between the landing pads and the binding arms.

In certain embodiments, when the amplifier probe is the secondary amplifier probe then the first and the second binding arm together comprise a sequence that is reverse complementary to the landing pad 1 and/or the landing pad 2 of the tertiary amplifier probe. In certain embodiments, when the amplifier probe is the secondary amplifier probe then the sequence of each of the binding arms is reverse complementary to the sequence of the amplifier-specific oligonucleotide of the primary clampFISH probe.

In certain embodiments, wherein when amplifier probe is the tertiary amplifier probe then the sequence of the first and the second binding arm together comprise a sequence that is reverse complementary to the landing pad 1 and/or the landing pad 2 of the secondary amplifier probe.

In certain embodiments, the 5′ end of the amplifier probe comprises as alkyne moiety and the 3′ end of the amplifier probe comprises an azide moiety. In certain embodiments the azide moiety is an N6-(6-Azido) hexyl-dATP. In certain embodiments, the alkyne moiety is hexynyl.

In certain embodiments, the 5′ end of the amplifier probe and the 3′ end of the amplifier probe can be covalently locked to form a circular amplifier probe

In certain embodiments, the 3′ end and the 5′ end of the amplifier probe do not comprise azide and alkyne moieties. In certain embodiments, circularization of the clampFISH probe is facilitated by a DNA ligase.

In certain embodiments, the amplifier probe is labeled with a fluorophore. In certain embodiments, the amplifier probe is not labeled with a fluorophore.

In certain embodiments, the secondary amplifier probe is one selected from SEQ ID NO: 423 to SEQ ID NO: 437.

In certain embodiments, the tertiary amplifier probe is one selected from SEQ ID NO: 438 to SEQ ID NO: 452.

### Read Out Probes

In certain embodiments, a readout probe is designed to bind in the center of 30 nucleotides long “landing pad” sequence of the amplifier probe. In certain embodiments the length of the readout probe was chosen such that the Gibbs free energy of binding to their target amplifier probe was −22 kcal/mol or −24 kcal/mol. In certain embodiments, the length of the readout probe is about 12 to about 25 nucleotides. In certain embodiments, the readout probe is about 20 nucleotides in length. In certain embodiments, the readout probe is designed to be easily strippable/removable from the amplifier probe to which it is bound. In certain embodiments, the readout probe can be removed using, for example, a denaturing agent such as Formamide or an increased temperature. In certain embodiments, the readout probe is coupled to a fluorescent label such as, for example, an NHS-ester dye. In certain the fluorescent label is, for example, Atto 488, AD 488-31; Cy3, Sigma-Aldrich-GEPA23001; Alexa Fluor 594, ThermoFisher-A20004; or Atto 647N, or AD 647N-31.

In certain embodiments, the readout probe is one selected from SEQ ID NO: 358 to SEQ ID NO: 392

### Methods

The present invention generally relates to click-amplifying FISH (clampFISH) methods for labeling, amplifying the labeling and reliably detecting one or more target nucleic acids in a sample. The present invention may be utilized in any FISH application known in the art. For example, the present invention may be used in methods to detect the presence of a target sequence, the location of a target sequence etc. The methods of the invention can be generally described as follow.

In one aspect the invention provides a method of exponentially amplifying the signal of a primary click-amplifying FISH (clampFISH) probe. In another aspect, the invention provides a method of detecting a fluorescently labeled target nucleic acid in a sample.

In certain embodiments, the method comprises: (a) hybridizing the primary clampFISH probe to a target nucleic acid in a sample; (b) contacting the primary clampFISH probe with a secondary amplifier probe; (c) adding a click chemistry agent that circularizes the primary clampFISH probe and covalently locks the secondary amplifier probe to the amplifier-specific oligonucleotide of the primary clampFISH probe to form a secondary sample; (d) contacting the secondary sample with a set of tertiary amplifier probes that bind to each secondary amplifier probe and adding a click chemistry agent that covalently locks the set of tertiary amplifier probes to each secondary amplifier probe to form a tertiary sample; (e) contacting the tertiary sample with a set of secondary amplifier probes that bind to each tertiary amplifier probe and adding a click chemistry agent that covalently locks the secondary amplifier probes to each tertiary amplifier probe; and, (f) repeating steps (d) and (e) until a desired amplified scaffold is achieved; (g) hybridizing a fluorescent dye-coupled DNA readout probe to the secondary or tertiary amplifier probes of the scaffold (h) detecting the signal from the readout probes by a fluorescence microscopy and/or flow cytometry.

In certain embodiments, optionally, the readout probe is removed/stripped from the secondary and the tertiary amplifier probes of the scaffold. In certain embodiments, optionally, once the readout probe is removed, a different readout probe is hybridized to the secondary or tertiary amplifier probes of the scaffold for signal detection using fluorescence microscopy and/or flow cytometry. In certain embodiments, the steps of stripping and hybridizing different readout probes is repeated any desired number of times.

In certain embodiments, the circularization of the primary clampFISH probe via click chemistry occurs with the aid of a circularizer oligonucleotide.

Alternatively, in certain embodiments, the amplifier probes are labeled with the fluorophores and therefore, step (g) is not required and the signal is detected directly from the labeled probes.

In certain embodiments, the primary clampFISH probe, the secondary amplifier probes and the tertiary amplifier probes are as described elsewhere herein.

In certain embodiments, the step (f) is repeated 1, 2, 3, 4, 5, 6, 7, 8, 9 or 10 times. In certain embodiments, the set of secondary and tertiary amplifier probes comprises at least 2 probes.

In certain embodiments, the click chemistry agent catalyzes an azide-alkyne cycloaddition thereby circularizing the primary clampFISH probe and covalently locking the secondary and the tertiary amplifier probes around their respective nucleic acid target.

In certain embodiments, the click chemistry is catalyzed by copper (I), copper (II) or ruthenium.

In certain embodiments, the primary clampFISH probe, the secondary amplifier probes and the tertiary amplifier probes are all DNA probes. In certain other embodiments, the primary clampFISH probe, the secondary amplifier probes and the tertiary amplifier probes are one selected from the group consisting of RNA, phosphorothioate DNA, peptide nucleic acid (PNA), locked nucleic acid (LNA), and 2′-O-Methyl RNA probes.

In certain embodiments, the target nucleic acid is a DNA or a RNA. In wherein the RNA is selected from the group consisting of messenger RNA, intronic RNA, exonic RNA, and non-coding RNA.

In certain embodiments, the tertiary amplifier probe has the same sequence as that of the secondary amplifier probe. In certain embodiments, the tertiary amplifier probe has a different sequence from that of the secondary amplifier probe.

In another aspect the invention provides a method for of synthesizing a primary clampFISH probe by ligating a first oligonucleotide to a second oligonucleotide using a ligase, wherein the first oligonucleotide and the second oligonucleotide are as described elsewhere herein. In certain embodiments, the 5′ end of the first nucleotide is ligated to the 3′ end of the second nucleotide using a ligase to form a primary clampFISH probe having a total length of about 109 nucleotides. In certain embodiments, the primary clampFISH probe is circularized by covalently locking the 3′ end of the first oligonucleotide to the 5′ end of the second oligonucleotide via click chemistry.

In certain embodiments, the first oligonucleotide the second oligonucleotide do not comprise azide and alkyne modifications. In certain embodiments, circularization of the clampFISH probe is facilitated using a ligase such as a DNA-ligase. In certain embodiments, the first and the second oligonucleotides are modified to comprise biotin and streptavidin, respectively, (or vice versa), and circularization of the clampFISH probe is facilitated via biotin-streptavidin interactions.

In certain embodiments, the method allows simultaneous detection of multiple target nucleic acids present in the sample. In certain embodiments, the method allows detection of lowly-expressed genes. In certain embodiments, the method allows detection of target nucleic acids using low-power air objective lenses. In certain embodiments, the method allows high-throughput detection of nucleic acids.

### Kits

In another aspect, the invention provides a kit comprising a set of primary click-amplifying FISH (clampFISH) probes, a set secondary amplifier probes, a set of tertiary amplifier probes, a set of amplifier-specific oligonucleotides, a set dye-coupled DNA readout probes, a ligase, a hybridization solution, and a click chemistry agent for signal amplification and detection of nucleic acids in a sample and instructions for use thereof.

In certain embodiments, the primary click-amplifying FISH (clampFISH) probe is as described elsewhere herein. In certain embodiments, the secondary amplifier probes, the tertiary amplifier probes, the dye-coupled DNA readout probes and the click chemistry agents are as described elsewhere herein.

### Target Nucleic Acid Sample

As contemplated herein, the present invention may be used in the analysis of sample for which nucleic acid analysis may be applied, as would be understood by those having ordinary skill in the art. For example, in one embodiment, the sample comprises at least one target nucleic acid, whose presence, location, or amount is desired to be investigated. For example, in certain embodiments, the nucleic acid can be mRNA. However, it should be appreciated that there is no limitation to the type of nucleic acid sample, which may include without limitation, any type of RNA, cDNA, genomic DNA, fragmented RNA or DNA and the like. In certain embodiments, the nucleic acid sample comprises at least one of messenger RNA, intronic RNA, exonic DNA, and non-coding RNA. The nucleic acid may be prepared for hybridization according to any manner as would be understood by those having ordinary skill in the art. It should also be appreciated that the sample may be an isolated nucleic acid sample, or it may form part of a lysed cell, or it may be an intact living cell. Samples may further be individual cells, or a population of cells, such as a population of cells corresponding to a particular tissue. Samples may also be a tissue section. It should be appreciated that there is no limitation to the size or type of sample, provided the sample includes at least one nucleic acid therein. For example, the sample may be derived or obtained from one or more eukaryotic cells, prokaryotic cells, bacteria, virus, exosome, liposome, and the like. In certain embodiments, a sample is fixed. For example, in one embodiment, a living cell or tissue is provided and fixed prior to application of one or more probes. In one embodiment, the sample is fixed using a crosslinking fixative (such as an aldehyde-based fixative). In other embodiments, the sample is fixed using a non-crosslinking fixative (such as an alcohol-based fixative).

### Click Chemistry

The present exponential fluorescent amplification of nucleic acids, via the clampFISH probes, circumvent enzyme-based amplification schemes by relying on a series of click chemistry reactions which are key for this invention.

In one embodiment, a click chemistry agent connects the 3′ and 5′ azide/alkyne ends of the primary, secondary and tertiary clampFISH probes around their respective nucleic acid target. In one embodiment, the click chemistry is catalyzed by a copper (I), a copper (II) or a ruthenium.

Azides and terminal alkynes can undergo Copper (I)-catalyzed Azide-Alkyne Cycloaddition (CuAAC) at room temperature. In this type of cycloaddition, also known as click chemistry, organic azides and terminal alkynes react to give 1,4-regioisomers of 1,2,3-triazoles. Examples of “click” chemistry reactions are described by Sharpless et al. (U.S. patent application U.S. Ser. No. 10/516,671), which developed reagents that react with each other in high yield and with few side reactions in a heteroatom linkage (as opposed to carbon-carbon bonds) in order to create libraries of chemical compounds. As described herein, click chemistry is used in the methods for labeling nucleic acids.

In some embodiments, the copper used as a catalyst for the click chemistry reaction is in the Cu (I) reduction state. This cycloaddition can also be conducted in the presence of a metal catalyst and a reducing agent. In certain embodiments, copper can be provided in the Cu (II) reduction state (for example, as a salt, such as but not limited to Cu(NO₃)₂Cu(OAc)₂ or CuSO₄), in the presence of a reducing agent wherein Cu (I) is formed in situ by the reduction of Cu (II). Such reducing agents include, but are not limited to, ascorbate, Tris(2-Carboxyethyl) Phosphine (TCEP), 2,4,6-trichlorophenol (TCP), NADH, NADPH, thiosulfate, metallic copper, quinone, hydroquinone, vitamin K₁, glutathione, cysteine, 2-mercaptoethanol, dithiothreitol, Fe²⁺, Co²⁺, or an applied electric potential. In other embodiments, the reducing agents include metals selected from Al, Be, Co, Cr, Fe, Mg, Mn, Ni, Zn, Au, Ag, Hg, Cd, Zr, Ru, Fe, Co, Pt, Pd, Ni, Rh, and W. In other embodiments, the copper used as a catalyst for the click chemistry reaction is in the Cu (II) state and is reduced to Cu (I) with sodium ascorbate.

The present copper-catalyzed azide-alkyne cycloadditions for labeling nucleic acids can be performed in water and a variety of solvents, including mixtures of water and a variety of (partially) miscible organic solvents including alcohols, dimethyl sulfoxide (DMSO), dimethyl formamide (DMF), tert-butanol (tBuOH) and acetone.

Certain metal ions are unstable in aqueous solvents, by way of example Cu (I), therefore stabilizing ligands/chelators can be used to improve the reaction. In certain embodiments at least one copper chelator is used in the methods described herein, wherein such chelators bind copper in the Cu (I) state. In certain embodiments at least one copper chelator is used in the methods described herein. In certain embodiments, the copper (I) chelator is a 1,10 phenanthroline-containing copper (I) chelator. Non-limiting examples of such phenanthroline-containing copper (I) chelators include, but are not limited to, bathophenanthroline disulfonic acid (4,7-diphenyl-1,10-phenanthroline disulfonic acid) and bathocuproine disulfonic acid (BCS; 2,9-dimethyl-4,7-diphenyl-1,10-phenanthroline disulfonate). Other chelators used in such methods include, but are not limited to, N-(2-acetamido)iminodiacetic acid (ADA), pyridine-2,6-dicarboxylic acid (PDA), S-carboxymethyl-L-cysteine (SCMC), trientine, tetra-ethylenepolyamine (TEPA), NNNN-tetrakis (2-pyridylmethyl)ethylenediamine (TPEN), EDTA, neocuproine, N-(2-acetamido)iminodiacetic acid (ADA), pyridine-2,6-dicarboxylic acid (PDA), S-carboxymethyl-L-cysteine (SCMC), tris-(benzyl-triazolylmethyl)amine (TBTA), or a derivative thereof. Most metal chelators, a wide variety of which are known in the art, are known to chelate several metals, and thus metal chelators in general can be tested for their function in 1,3 cycloaddition reactions catalyzed by copper. In certain embodiments, histidine is used as a chelator, while in other embodiments glutathione is used as a chelator and a reducing agent.

The concentration of the reducing agents used in the “click” chemistry reaction described herein can be in the micromolar to millimolar range. In certain embodiments the concentration of the reducing agent is from about 100 micromolar to about 100 millimolar. In other embodiments the concentration of the reducing agent is from about 10 micromolar to about 10 millimolar. In other embodiments the concentration of the reducing agent is from about 1 micromolar to about 1 millimolar. In yet other embodiments, the concentration of the reducing agent is 2.5 millimolar.

The concentration of a copper chelator used in the “click” chemistry reaction described herein can be determined and optimized using methods well known in the art. In certain embodiments, the chelator concentrations used in the methods described herein is in the micromolar to millimolar range, by way of example only, from 1 micromolar to 100 millimolar.

In certain embodiments the chelator concentration is from about 10 micromolar to about 10 millimolar. In other embodiments the chelator concentration is from about 50 micromolar to about 10 millimolar. In other embodiments the chelator, can be provided in a solution that includes a water miscible solvent such as, alcohols, dimethyl sulfoxide (DMSO), dimethyl formamide (DMF), tert-butanol (tBuOH) and acetone. In other embodiments the chelator, can be provided in a solution that includes a solvent such as, for example, dimethyl sulfoxide (DMSO) or dimethylformamide (DMF).

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Materials and Methods

clampFISH 2.0 Primary Probe Design and Construction clampFISH 2.0 primary probes were constructed as follows. First, a set of 30mer RNA-targeting probe sequences were designed for each target gene with custom MATLAB software, as previously described, and added a flanking 10 mer 5′ sequence (AAGTGACTGT) (SEQ ID NO: 552) and a 10mer 3′ sequence (ACATCATAGT) (SEQ ID NO: 553) to each of those respective ends were designed, producing a 50 mer sequence. The 50mer sequences were run through a custom MATLAB script using BLAST (Camacho et al. 2009) for alignment to the human transcriptome and NUPACK (Dirks and Pierce 2004; Dirks et al. 2007; Dirks and Pierce 2003; Fornace, Porubsky, and Pierce 2020) to predict binding energies of the off-target transcriptomic hits. Only the hits with binding energy less than-14 kcal/mol were kept, and then each of these hits were assigned with the maximum fragments per kilobase of transcript per million (FPKM) from a set of 13 human RNA-seq datasets from the ENCODE portal (Davis et al. 2018; ENCODE Project Consortium 2012) (encodeproject.org). For each gene, 24-32 primary probes per gene target were selected, with a preference for probes targeting the coding region and where the sum of FPKM values from its predicted off-target hits was minimized. For probes targeting GFP, 10 probes whose 30mer primary probe sequences were taken from Rouhanifard et al. 2018 were used. The 50mer sequences were ordered from Integrated DNA Technologies (IDT) and pooled together for a given gene. For each gene-specific pool, an azido-dATP (N6-(6-Azido) hexyl-3′-dATP, Jena Bioscience, NU-1707L) was added to the probes' 3′ ends with Terminal Transferase (New England Biolabs, M0315L), which adds a single azido-dATP molecule. Then, the 5′ ends were phosphorylated with T4 Polynucleotide Kinase (New England Biolabs, M0201L). Each gene-specific pool of 51mer oligonucleotides was mixed with a 20mer ligation adapter (ACAGTCACTTCAACACTCAG) (SEQ ID NO: 554) and a 58mer oligonucleotide, which were both ordered from IDT. The 58mer oligonucleotide was ordered with a 5′ alkyne modification (5′ hexynyl) and was designed with the following sequences, in 5′ to 3′ order: a universal 18mer sequence (AGACATTCTCGTCAAGAT) (SEQ ID NO: 550), an amplifier-specific 30mer sequence (serving as a landing pad upon which a secondary probe can bind), and a universal 10mer sequence (CTGAGTGTTG) (SEQ ID NO: 551). Then, T7 DNA Ligase (New England Biolabs, M0318L) was added, ligating together a complete 109mer (50+1+58) primary probe. Then ammonium acetate was added to a 2.5M concentration, centrifuged twice at 17,000 g where each time all but the bottom 20 μL of solution was pipetted to a new tube, ethanol precipitated the probes, resuspended the probes in nuclease-free water, centrifuged the tube at 17,000 g, and pipetted all but the bottom 5 μL into a new tube.

TABLE 1A
clampFISH 2.0 RNA-targeting oligos

| | | | | number of | | |
|---|---|---|---|---|---|---|
| clampFISH 2.0 | | | | probes on | | |
| RNA-targeting | | | | each | | |
| oligos information | | | number | gene region | | |
| | RefSeq | genome | of | | 3′ | 5′ |
| targeted | Number | build | probes | CDS | UTR | UTR |
| AXL | NM_001278599.1 | hg19 | 30 | 11 | 19 | 0 |
| ITGA3 | NM_002204.4 | hg38 | 32 | 24 | 8 | 0 |
| EGFR | NM_001346897.1 | hg19 | 30 | 30 | 0 | 0 |
| WNT5A | NM_003392.4 | hg38 | 32 | 8 | 24 | 0 |
| MMP1 | NM_001145938.2 | hg38 | 24 | 21 | 3 | 0 |
| NGFR | NM_002507.4 | hg38 | 24 | 10 | 14 | 0 |
| FN1 | NM_054034.2 | hg38 | 32 | 31 | 1 | 0 |
| DDX58 | NM_014314.4 | hg38 | 32 | 32 | 0 | 0 |
| MITF | NM_198159.3 | hg38 | 32 | 22 | 10 | 0 |
| UBC | NM_021009.7 | hg38 | 32 | 31 | 0 | 1 |
| GFP | | | 10 | | | |

TABLE 1B
clampFISH 2.0 RNA-targeting oligos sequences

| Gene | | |
|---|---|---|
| targeted | Oligo name | Oligo sequence |
| AXL | AXL_CDS_1 | aagtgactgtCGGATGTGATAAGGGGTGTGAGGATGGAGG |
| | | acatcatagt(SEQ ID NO: 1) |
| AXL | AXL_CDS_2 | aagtgactgtCATTCCGCGTAGCACTAATGTTCTCAGGGGac |
| | | atcatagt(SEQ ID NO: 2) |
| AXL | AXL_CDS_3 | aagtgactgtTGCCACACACACTGTCAGATTGGACACAGAa |
| | | catcatagt(SEQ ID NO: 3) |
| AXL | AXL_CDS_4 | aagtgactgtACGACTGCTCCTAGCAGTACATACCACCAGa |
| | | catcatagt(SEQ ID NO: 4) |
| AXL | AXL_CDS_6 | aagtgactgtAAGGTAGCTTCAGTGGTCCGACGACTGTAGa |
| | | catcatagt(SEQ ID NO: 5) |
| AXL | AXL_CDS_8 | aagtgactgtCAGATGGCAATCTTCATCGTCTTCACAGCCac |
| | | atcatagt(SEQ ID NO: 6) |
| AXL | AXL_CDS_11 | aagtgactgtTGGAAACAGACACCGATGAGCCTCATGACGa |
| | | catcatagt(SEQ ID NO: 7) |
| AXL | AXL_CDS_13 | aagtgactgtGTCTCCATGTTTCATGAAAGGTAAGATGACac |
| | | atcatagt(SEQ ID NO: 8) |
| AXL | AXL_CDS_20 | aagtgactgtGTGTAGACACGGTCAGCTAGACTCTCAATGa |
| | | catcatagt(SEQ ID NO: 9) |
| AXL | AXL_CDS_22 | aagtgactgtGACATCAAGGCATACAGTCCATCCAGACAGa |
| | | catcatagt(SEQ ID NO: 10) |
| AXL | AXL_CDS_25 | aagtgactgtTGAGGCAGCTACAGGAATCCTTAGGGTCTGa |
| | | catcatagt(SEQ ID NO: 11) |
| AXL | AXL_3UTR_4 | aagtgactgtCCTTTGAATCTAGACTCTTTAAACCTTGGAac |
| | | atcatagt(SEQ ID NO: 12) |
| AXL | AXL_3UTR_5 | aagtgactgtACCAAAGACTCACAGCATCTTTGAAACCTAa |
| | | catcatagt(SEQ ID NO: 13) |
| AXL | AXL_3UTR_6 | aagtgactgtGAATTAGAGACTTTGGAATTTCAGGTCCTTac |
| | | atcatagt(SEQ ID NO: 14) |
| AXL | AXL_3UTR_7 | aagtgactgtAATGTCAGAGCTTTAGAACAGTAGGTCTTAac |
| | | atcatagt(SEQ ID NO: 15) |
| AXL | AXL_3UTR_8 | aagtgactgtGAACTTTAGAACCTAAAACCTTGAGGCTTTac |
| | | atcatagt(SEQ ID NO: 16) |
| AXL | AXL_3UTR_9 | aagtgactgtATCTAGACTCATAGAGCCTTAGCTCCTAAAac |
| | | atcatagt(SEQ ID NO: 17) |
| AXL | AXL_3UTR_10 | aagtgactgtTCTTACATTTTAAGGACTCTGAACTCTAGAac |
| | | atcatagt(SEQ ID NO: 18) |
| AXL | AXL_3UTR_11 | aagtgactgtCCTCCATGTCTAGAACTATAGAATCTTTAGac |
| | | atcatagt(SEQ ID NO: 19) |
| AXL | AXL_3UTR_12 | aagtgactgtAACATCACATTTTAGAATCCTAGGCCTTAGac |
| | | atcatagt(SEQ ID NO: 20) |
| AXL | AXL_3UTR_14 | aagtgactgtAACATTTTGAAGCCTTATGATCTAGAGCCTac |
| | | atcatagt(SEQ ID NO: 21) |
| AXL | AXL_3UTR_15 | aagtgactgtGCCTCAGAAACTATAATTGATCATCATTAGac |
| | | atcatagt(SEQ ID NO: 22) |
| AXL | AXL_3UTR_16 | aagtgactgtTTCGACCCTTAGGATCTAGGATCTTATACAac |
| | | atcatagt(SEQ ID NO: 23) |
| AXL | AXL_3UTR_18 | aagtgactgtAGAATATCACATCTTAGAACACCGGACCTTac |
| | | atcatagt(SEQ ID NO: 24) |
| AXL | AXL_3UTR_21 | aagtgactgtCAATGGAATCTAGAGTGCCTTGAAACAGAAa |
| | | catcatagt(SEQ ID NO: 25) |
| AXL | AXL_3UTR_22 | aagtgactgtAACTTAGATGCTTAGGATCCGGAATCTTGGac |
| | | atcatagt(SEQ ID NO: 26) |
| AXL | AXL_3UTR_25 | aagtgactgtAATGTGGATTCTTGGACTCTTAACCATGGTac |
| | | atcatagt(SEQ ID NO: 27) |
| AXL | AXL_3UTR_26 | aagtgactgtCATTTGAGTCTTAGAACTACAGTGCCTAGAac |
| | | atcatagt(SEQ ID NO: 28) |
| AXL | AXL_3UTR_27 | aagtgactgtGTCTAGACCTGTGGACCTTTAGAATCTTAGac |
| | | atcatagt(SEQ ID NO: 29) |
| AXL | AXL_3UTR_28 | aagtgactgtGTATAGGGTTAGAACCTTGAAATTGCACCTac |
| | | atcatagt(SEQ ID NO: 30) |
| EGFR | EGFR_CDS_1 | aagtgactgtTGTTACTCGTGCCTTGGCAAACTTTCTTTTacat |
| | | catagt(SEQ ID NO: 31) |
| EGFR | EGFR_CDS_2 | aagtgactgtATGATCTTCAAAAGTGCCCAACTGCGTGAGa |
| | | catcatagt(SEQ ID NO: 32) |
| EGFR | EGFR_CDS_3 | aagtgactgtCATAGGTAATTTCCAAATTCCCAAGGACCAac |
| | | atcatagt(SEQ ID NO: 33) |
| EGFR | EGFR_CDS_4 | aagtgactgtCAGGTTTTCCAAAGGAATTCGCTCCACTGTac |
| | | atcatagt(SEQ ID NO: 34) |
| EGFR | EGFR_CDS_5 | aagtgactgtGGAATTTTCGTAGTACATATTTCCTCTGATaca |
| | | tcatagt(SEQ ID NO: 35) |
| EGFR | EGFR_CDS_6 | aagtgactgtGCATCATAGTTAGATAAGACTGCTAAGGCAa |
| | | catcatagt(SEQ ID NO: 36) |
| EGFR | EGFR_CDS_10 | aagtgactgtTCCTTGCACGTGGCTTCGTCTCGGAATTTGaca |
| | | tcatagt(SEQ ID NO: 37) |
| EGFR | EGFR_CDS_11 | aagtgactgtCTGGTACGTGGTGGGGTTGTAGAGCATGAGa |
| | | catcatagt(SEQ ID NO: 38) |
| EGFR | EGFR_CDS_13 | aagtgactgtACATAATTACGGGGACACTTCTTCACGCAGac |
| | | atcatagt(SEQ ID NO: 39) |
| EGFR | EGFR_CDS_14 | aagtgactgtATTCACCAATACCTATTCCGTTACACACTTaca |
| | | tcatagt(SEQ ID NO: 40) |
| EGFR | EGFR_CDS_15 | aagtgactgtATTCGTAGCATTTATGGAGAGTGAGTCTTTac |
| | | atcatagt(SEQ ID NO: 41) |
| EGFR | EGFR_CDS_16 | aagtgactgtCACTGATGGAGGTGCAGTTTTTGAAGTGTTac |
| | | atcatagt(SEQ ID NO: 42) |
| EGFR | EGFR_CDS_17 | aagtgactgtATGTGTGAAGGAGTCACCCCTAAATGCCACa |
| | | catcatagt(SEQ ID NO: 43) |
| EGFR | EGFR_CDS_19 | aagtgactgtAAAACCCTGTGATTTCCTTTACGGTTTTCAaca |
| | | tcatagt(SEQ ID NO: 44) |
| EGFR | EGFR_CDS_21 | aagtgactgtCGCGTATGATTTCTAGGTTCTCAAAGGCATac |
| | | atcatagt(SEQ ID NO: 45) |
| EGFR | EGFR_CDS_22 | aagtgactgtAAGAGAAAACTGACCATGTTGCTTGGTCCTac |
| | | atcatagt(SEQ ID NO: 46) |
| EGFR | EGFR_CDS_24 | aagtgactgtTATCACATCTCCATCACTTATCTCCTTGAGaca |
| | | tcatagt(SEQ ID NO: 47) |
| EGFR | EGFR_CDS_26 | aagtgactgtCACTGTATGCACTCAGAGTTCTCCACAAACac |
| | | atcatagt(SEQ ID NO: 48) |
| EGFR | EGFR_CDS_31 | aagtgactgtCGAACGATGTGGCGCCTTCGCATGAAGAGGa |
| | | catcatagt(SEQ ID NO: 49) |
| EGFR | EGFR_CDS_33 | aagtgactgtTGAATTCAGTTTCCTTCAAGATCCTCAAGAac |
| | | atcatagt(SEQ ID NO: 50) |
| EGFR | EGFR_CDS_35 | aagtgactgtTCCTTGATAGCGACGGGAATTTTAACTTTCac |
| | | atcatagt(SEQ ID NO: 51) |
| EGFR | EGFR_CDS_36 | aagtgactgtTGTTGGCTTTCGGAGATGTTGCTTCTCTTAaca |
| | | tcatagt(SEQ ID NO: 52) |
| EGFR | EGFR_CDS_37 | aagtgactgtTGGCCATCACGTAGGCTTCATCGAGGATTTac |
| | | atcatagt(SEQ ID NO: 53) |
| EGFR | EGFR_CDS_39 | aagtgactgtAATATTGTCTTTGTGTTCCCGGACATAGTCaca |
| | | tcatagt(SEQ ID NO: 54) |
| EGFR | EGFR_CDS_41 | aagtgactgtCTTCTGCATGGTATTCTTTCTCTTCCGCACacat |
| | | catagt(SEQ ID NO: 55) |
| EGFR | EGFR_CDS_42 | aagtgactgtAATTGATTCCAATGCCATCCACTTGATAGGac |
| | | atcatagt(SEQ ID NO: 56) |
| EGFR | EGFR_CDS_48 | aagtgactgtAACTTTGGGCGACTATCTGCGTCTATCATCac |
| | | atcatagt(SEQ ID NO: 57) |
| EGFR | EGFR_CDS_49 | aagtgactgtTCGGGCCATTTTGGAGAATTCGATGATCAAac |
| | | atcatagt(SEQ ID NO: 58) |
| EGFR | EGFR_CDS_50 | aagtgactgtAATGCATTCTTTCATCCCCCTGAATGACAAac |
| | | atcatagt(SEQ ID NO: 59) |
| EGFR | EGFR_CDS_52 | aagtgactgtAGCTTTGCAGCCCATTTCTATCAATGCAAGac |
| | | atcatagt(SEQ ID NO: 60) |
| ITGA3 | ITGA3_CDS_1 | aagtgactgtACTACCAGGAATCGGGTATCCAGGTTGAAGa |
| | | catcatagt(SEQ ID NO: 61) |
| ITGA3 | ITGA3_CDS_3 | aagtgactgtCAGGGTCATTTTTCACTGTGATGTTCATCCaca |
| | | tcatagt(SEQ ID NO: 62) |
| ITGA3 | ITGA3_CDS_4 | aagtgactgtTCCAAGCCACATGTCCTCAATAATGTGATGac |
| | | atcatagt(SEQ ID NO: 63) |
| ITGA3 | ITGA3_CDS_5 | aagtgactgtTCTAGGTCATTGCCTCGCACGTAGCACTTGac |
| | | atcatagt(SEQ ID NO: 64) |
| ITGA3 | ITGA3_CDS_6 | aagtgactgtTTGTGGTAGGTCTGCCAGTCATCACTGGAGac |
| | | atcatagt(SEQ ID NO: 65) |
| ITGA3 | ITGA3_CDS_7 | aagtgactgtTCCAGGTAGTCTGTGTTGCTATTGCACATCac |
| | | atcatagt(SEQ ID NO: 66) |
| ITGA3 | ITGA3_CDS_8 | aagtgactgtTGAATCATGTAGCTGTTTCCTTTCCAGTTGaca |
| | | tcatagt(SEQ ID NO: 67) |
| ITGA3 | ITGA3_CDS_9 | aagtgactgtTGTAACTATACTCAGATAAGTCCCACTCCTac |
| | | atcatagt(SEQ ID NO: 68) |
| ITGA3 | ITGA3_CDS_10 | aagtgactgtAATATAGAGGTTTCCTTGGTCCTCTGGGTCac |
| | | atcatagt(SEQ ID NO: 69) |
| ITGA3 | ITGA3_CDS_11 | aagtgactgtAGGATGAAGCTGCCTACCTGCATCGTGTACac |
| | | atcatagt(SEQ ID NO: 70) |
| ITGA3 | ITGA3_CDS_12 | aagtgactgtCACCTGTCACAATGGTGATGTTTTTGGGGTac |
| | | atcatagt(SEQ ID NO: 71) |
| ITGA3 | ITGA3_CDS_15 | aagtgactgtATGCTGGCCACAGATAAACCAAAGGCAGAG |
| | | acatcatagt(SEQ ID NO: 72) |
| ITGA3 | ITGA3_CDS_16 | aagtgactgtAGAGCTACTGTGATAGATGTACACTTTGCCac |
| | | atcatagt(SEQ ID NO: 73) |
| ITGA3 | ITGA3_CDS_19 | aagtgactgtCACCAAGGTCTTGTGGACGATGTTGATGACac |
| | | atcatagt(SEQ ID NO: 74) |
| ITGA3 | ITGA3_CDS_21 | aagtgactgtCAGCTCGCAAAAGATGGTCTCATTAGCTTGac |
| | | atcatagt(SEQ ID NO: 75) |
| ITGA3 | ITGA3_CDS_24 | aagtgactgtAAAGAAGCTTTGTAGCCGGTGATTTACCATac |
| | | atcatagt(SEQ ID NO: 76) |
| ITGA3 | ITGA3_CDS_25 | aagtgactgtTACATCCTCCACAGTTTTCATGCCAGACTCac |
| | | atcatagt(SEQ ID NO: 77) |
| ITGA3 | ITGA3_CDS_26 | aagtgactgtATGGACGGTGATCTCCGTGGGATACAGCAGa |
| | | catcatagt(SEQ ID NO: 78) |
| ITGA3 | ITGA3_CDS_27 | aagtgactgtGGTCAGAAAGAGTGAGGTTGAGAGGGTTGA |
| | | acatcatagt(SEQ ID NO: 79) |
| ITGA3 | ITGA3_CDS_28 | aagtgactgtGTCAGCACAGTCTCAGACTTGGCTTTTTTGac |
| | | atcatagt(SEQ ID NO: 80) |
| ITGA3 | ITGA3_CDS_29 | aagtgactgtCAGTCACGTTGGTGACAACGGGGGCATCAGa |
| | | catcatagt(SEQ ID NO: 81) |
| ITGA3 | ITGA3_CDS_31 | aagtgactgtTTTACCCGGACTCGGTCAAAGTCTCTGTAAac |
| | | atcatagt(SEQ ID NO: 82) |
| ITGA3 | ITGA3_CDS_33 | aagtgactgtTCAATGTCCACAGAGAACCACGTGGTCTTGac |
| | | atcatagt(SEQ ID NO: 83) |
| ITGA3 | ITGA3_3UTR_1 | aagtgactgtGTGGTACTTGGGCATGATCTGATAATAGCGac |
| | | atcatagt(SEQ ID NO: 84) |
| ITGA3 | ITGA3_3UTR_2 | aagtgactgtAGGACGTCAGTAGTATTGGTCCCGAGTCTGac |
| | | atcatagt(SEQ ID NO: 85) |
| ITGA3 | ITGA3_3UTR_3 | aagtgactgtCATGTGCATGCTCTTGACGAGGTGTATGTGac |
| | | atcatagt(SEQ ID NO: 86) |
| ITGA3 | ITGA3_3UTR_4 | aagtgactgtACAGGAATGCAGTGCACTCGGCGTTGTAAGa |
| | | catcatagt(SEQ ID NO: 87) |
| ITGA3 | ITGA3_3UTR_7 | aagtgactgtTTGACAGCAAAGTCAATCCAGTGACTGTGGa |
| | | catcatagt(SEQ ID NO: 88) |
| ITGA3 | ITGA3_3UTR_8 | aagtgactgtTTCTTTCTAGTTCCTTTGCTGTTGGGTCTGacat |
| | | catagt(SEQ ID NO: 89) |
| ITGA3 | ITGA3_3UTR_9 | aagtgactgtTATCCTCCCAACATCTGTCTGTCCAGAATTaca |
| | | tcatagt(SEQ ID NO: 90) |
| ITGA3 | ITGA3_3UTR_10 | aagtgactgtCTGGTAGTGGTGAGTGAGAAGTGGCATCTCa |
| | | catcatagt(SEQ ID NO: 91) |
| ITGA3 | ITGA3_3UTR_11 | aagtgactgtCTATTCTACATTCAAGTGTCGCTCCCTCCGaca |
| | | tcatagt(SEQ ID NO: 92) |
| WNT5 | WNT5A_CDS_1 | aagtgactgtGAAGAACTTGGAAGACATTGCACTTCCAGCa |
| A | | catcatagt(SEQ ID NO: 93) |
| WNT5 | WNT5A_CDS_3 | aagtgactgtGACCACCAAGAATTGGCTTCAATTACAACCa |
| A | | catcatagt(SEQ ID NO: 94) |
| WNT5 | WNT5A_CDS_4 | aagtgactgtTCTGACATCTGAACAGGGTTATTCATACCTac |
| A | | atcatagt(SEQ ID NO: 95) |
| WNT5 | WNT5A_CDS_6 | aagtgactgtGATGTACTGCATGTGGTCCTGATACAAGTGac |
| A | | atcatagt(SEQ ID NO: 96) |
| WNT5 | WNT5A_CDS_8 | aagtgactgtTGCTGCAGTTCCACCTTCGATGTCGGAATTac |
| A | | atcatagt(SEQ ID NO: 97) |
| WNT5 | WNT5A_CDS_9 | aagtgactgtCATCACCCTGCCAAAAACAGAGGTGTTATCa |
| A | | catcatagt(SEQ ID NO: 98) |
| WNT5 | WNT5A_CDS_10 | aagtgactgtTTGGCAAAGCGGTAGCCATAGTCGATGTTGa |
| A | | catcatagt(SEQ ID NO: 99) |
| WNT5 | WNT5A_CDS_11 | aagtgactgtTTCATGAGGATGCGAGCACTCTCGTAGGAGa |
| A | | catcatagt(SEQ ID NO: 100) |
| WNT5 | WNT5A_3UTR_ | aagtgactgtATGTGTACCATGTCCACACAGCTAAGTTAGac |
| A | 2 | atcatagt(SEQ ID NO: 101) |
| WNT5 | WNT5A_3UTR_ | aagtgactgtCTCAAATAGGTTGTCTGCTCTGGTGCAGTGac |
| A | 10 | atcatagt(SEQ ID NO: 102) |
| WNT5 | WNT5A_3UTR_ | aagtgactgtATGTTTCATTTCCTGTCCTACAACCAAAGGac |
| A | 13 | atcatagt(SEQ ID NO: 103) |
| WNT5 | WNT5A_3UTR_ | aagtgactgtTGTGAATTCCATTAGCCAAGTTCTGTCATTaca |
| A | 15 | tcatagt(SEQ ID NO: 104) |
| WNT5 | WNT5A_3UTR_ | aagtgactgtTGGGAACAGTTTTAAGGTACACCTGCAGTAa |
| A | 17 | catcatagt(SEQ ID NO: 105) |
| WNT5 | WNT5A_3UTR_ | aagtgactgtATGCTCTAGACAGAGAAATAACCCCAGAGTa |
| A | 18 | catcatagt(SEQ ID NO: 106) |
| WNT5 | WNT5A_3UTR_ | aagtgactgtTGTCTATAACGAACAAGTAATGCCCTCTCCac |
| A | 21 | atcatagt(SEQ ID NO: 107) |
| WNT5 | WNT5A_3UTR_ | aagtgactgtCATTTCTAATAGGCATGGGTTTCCATTCTGaca |
| A | 23 | tcatagt(SEQ ID NO: 108) |
| WNT5 | WNT5A_3UTR_ | aagtgactgtTAGTGGGCTGAATATTCCTTAGGGACTCAAac |
| A | 24 | atcatagt(SEQ ID NO: 109) |
| WNT5 | WNT5A_3UTR_ | aagtgactgtCACAGTTGGCTGAGGTGATATGAAAGCCAAa |
| A | 26 | catcatagt(SEQ ID NO: 110) |
| WNT5 | WNT5A_3UTR_ | aagtgactgtAGTGCTTTTTGCTTTCAAGATCTTTTGCTCacat |
| A | 28 | catagt(SEQ ID NO: 111) |
| WNT5 | WNT5A_3UTR_ | aagtgactgtCGTCATGGTTTCTCCAAAAATCTCCACATGac |
| A | 29 | atcatagt(SEQ ID NO: 112) |
| WNT5 | WNT5A_3UTR_ | aagtgactgtTCTTTTATTACTTTGACATGTAGTCATCCTacat |
| A | 30 | catagt(SEQ ID NO: 113) |
| WNT5 | WNT5A_3UTR_ | aagtgactgtAAACCTTTTGAAGCTTCCAAATAGCTTGGTac |
| A | 31 | atcatagt(SEQ ID NO: 114) |
| WNT5 | WNT5A_3UTR_ | aagtgactgtAGAGAATTCCCCTTTTGTTCCATTACATATaca |
| A | 32 | tcatagt(SEQ ID NO: 115) |
| WNT5 | WNT5A_3UTR_ | aagtgactgtAAATCTGGTCACGGATAAAAGCATGTGCCTa |
| A | 34 | catcatagt(SEQ ID NO: 116) |
| WNT5 | WNT5A_3UTR_ | aagtgactgtTCAAATAGAGAGGTGCAATAGTTGCAGTGGa |
| A | 35 | catcatagt(SEQ ID NO: 117) |
| WNT5 | WNT5A_3UTR_ | aagtgactgtAACCTTTAGTTTCCAACCAGTTTTCATTCTaca |
| A | 37 | tcatagt(SEQ ID NO: 118) |
| WNT5 | WNT5A_3UTR_ | aagtgactgtACTGTCCAGATTTCCGTGTACATTTTTGAAaca |
| A | 38 | tcatagt(SEQ ID NO: 119) |
| WNT5 | WNT5A_3UTR_ | aagtgactgtATGCATTCTTGGCATCCTTAAAAGGTTTCAac |
| A | 39 | atcatagt(SEQ ID NO: 120) |
| WNT5 | WNT5A_3UTR_ | aagtgactgtCTATATGTTGGTCTGCTGTTTTTTTGTGGAacat |
| A | 40 | catagt(SEQ ID NO: 121) |
| WNT5 | WNT5A_3UTR_ | aagtgactgtGTCCTAGAACCACAAGAGTTTGAATTTGCCac |
| A | 41 | atcatagt(SEQ ID NO: 122) |
| WNT5 | WNT5A_3UTR_ | aagtgactgtGCGTCATATTTGATAATTCTAGTGGCTTGAac |
| A | 42 | atcatagt(SEQ ID NO: 123) |
| WNT5 | WNT5A_3UTR_ | aagtgactgtACCGGTCTCTTATTCAGAGTATTTACTCTGaca |
| A | 43 | tcatagt(SEQ ID NO: 124) |
| FN1 | FN1_CDS_2 | aagtgactgtCTCCCACTGTTGATTTATCTGATAGTGTTTaca |
| | | tcatagt(SEQ ID NO: 125) |
| FN1 | FN1_CDS_3 | aagtgactgtACAAGTACAAACCAACGCATTGCCTAGGTAa |
| | | catcatagt(SEQ ID NO: 126) |
| FN1 | FN1_CDS_4 | aagtgactgtTACTCTCGCAGTTAAAACCTCGGCTTCCTCac |
| | | atcatagt(SEQ ID NO: 127) |
| FN1 | FN1_CDS_5 | aagtgactgtTCATAAGTGTCACCCACTCGGTAAGTGTTCac |
| | | atcatagt(SEQ ID NO: 128) |
| FN1 | FN1_CDS_6 | aagtgactgtTACAGTCCCAGATCATGGAGTCTTTAGGACac |
| | | atcatagt(SEQ ID NO: 129) |
| FN1 | FN1_CDS_7 | aagtgactgtCAGCGGTTTGCGATGGTACAGCTTATTCTCac |
| | | atcatagt(SEQ ID NO: 130) |
| FN1 | FN1_CDS_8 | aagtgactgtTCCAGGTGTCACCAATCTTGTAGGACTGACac |
| | | atcatagt(SEQ ID NO: 131) |
| FN1 | FN1_CDS_9 | aagtgactgtTAACATGTAACCACCAGTCTCATGTGGTCTac |
| | | atcatagt(SEQ ID NO: 132) |
| FN1 | FN1_CDS_12 | aagtgactgtTCTCCCACGTTTCTCCGACCACATAGGAAGac |
| | | atcatagt(SEQ ID NO: 133) |
| FN1 | FN1_CDS_13 | aagtgactgtCAAGTACAATCTACCATCATCCAGCCTTGGac |
| | | atcatagt(SEQ ID NO: 134) |
| FN1 | FN1_CDS_14 | aagtgactgtCTATTTCTAGAAGTGCAAGTGATGCGTCCGac |
| | | atcatagt(SEQ ID NO: 135) |
| FN1 | FN1_CDS_18 | aagtgactgtAGTAGACCACACCACTGTCTGTGACACAGTa |
| | | catcatagt(SEQ ID NO: 136) |
| FN1 | FN1_CDS_20 | aagtgactgtTAAGACACATGGCTCTCCATTTGAGTTGCCac |
| | | atcatagt(SEQ ID NO: 137) |
| FN1 | FN1_CDS_21 | aagtgactgtGAGTAGAACGTCCTGCCATTGTAGGTGAATa |
| | | catcatagt(SEQ ID NO: 138) |
| FN1 | FN1_CDS_22 | aagtgactgtAATTCGAAGTTGTGCTGCACCAAAGATGTCac |
| | | atcatagt(SEQ ID NO: 139) |
| FN1 | FN1_CDS_23 | aagtgactgtTGTGCAGAAAGAGTATTTCTGGTCCTGCTCac |
| | | atcatagt(SEQ ID NO: 140) |
| FN1 | FN1_CDS_24 | aagtgactgtAAGGCACCATTGGAATTTCCTCCTCGAGTCac |
| | | atcatagt(SEQ ID NO: 141) |
| FN1 | FN1_CDS_26 | aagtgactgtTCTTCTGCCCTCAGAAGTGCAATCAGTGTAac |
| | | atcatagt(SEQ ID NO: 142) |
| FN1 | FN1_CDS_27 | aagtgactgtTTCTGTGTGGTCCCACACCACTTCATGTTGaca |
| | | tcatagt(SEQ ID NO: 143) |
| FN1 | FN1_CDS_28 | aagtgactgtGGCAGAACCCAAACTTCTGGTCGGCATCATa |
| | | catcatagt(SEQ ID NO: 144) |
| FN1 | FN1_CDS_29 | aagtgactgtTACATGACCCCTTCATTGGTTGTGCAGATTac |
| | | atcatagt(SEQ ID NO: 145) |
| FN1 | FN1_CDS_30 | aagtgactgtTCATGCTGCTTATCCCACTGATCTCCAATGaca |
| | | tcatagt(SEQ ID NO: 146) |
| FN1 | FN1_CDS_32 | aagtgactgtAAGCTGCGAGTAGGCAATGCATGTCCATTCa |
| | | catcatagt(SEQ ID NO: 147) |
| FN1 | FN1_CDS_33 | aagtgactgtGTAAGTGATGTCATCAACAATGCACTGATCac |
| | | atcatagt(SEQ ID NO: 148) |
| FN1 | FN1_CDS_34 | aagtgactgtTTCATGACGCTTGTGGAATGTGTCGTTCACac |
| | | atcatagt(SEQ ID NO: 149) |
| FN1 | FN1_CDS_35 | aagtgactgtCTGACCGAAGCATGTACAGTTCAGCATGTGa |
| | | catcatagt(SEQ ID NO: 150) |
| FN1 | FN1_CDS_36 | aagtgactgtAAACGTCCCAGTCTCTGAATCCTGGCATTGac |
| | | atcatagt(SEQ ID NO: 151) |
| FN1 | FN1_CDS_37 | aagtgactgtACATACTTCTCCCATGAATCTCCAATTTGAac |
| | | atcatagt(SEQ ID NO: 152) |
| FN1 | FN1_CDS_38 | aagtgactgtCATAGCAGTAGCACTGGTATCTGACACCATac |
| | | atcatagt(SEQ ID NO: 153) |
| FN1 | FN1_CDS_40 | aagtgactgtGACTCGGAGTCTCAGTGATAAATACTTCGAac |
| | | atcatagt(SEQ ID NO: 154) |
| FN1 | FN1_CDS_42 | aagtgactgtATACTCACAGGTCTCCACCTGAGAATGTACac |
| | | atcatagt(SEQ ID NO: 155) |
| FN1 | FN1_3UTR_1 | aagtgactgtTAGGAATAGTTAATCAGAGTTGTTGGCTCAac |
| | | atcatagt(SEQ ID NO: 156) |
| DDX58 | DDX58_CDS_4 | aagtgactgtAAAGTCCAGAATAACCTGCATGGTCTAGGGa |
| | | catcatagt(SEQ ID NO: 157) |
| DDX58 | DDX58_CDS_5 | aagtgactgtCAGACAGATCAGAAATGATATCGGTTGGGAa |
| | | catcatagt(SEQ ID NO: 158) |
| DDX58 | DDX58_CDS_6 | aagtgactgtGCAAATCTGTAGAATTTCTTCACATTCCTGac |
| | | atcatagt(SEQ ID NO: 159) |
| DDX58 | DDX58_CDS_7 | aagtgactgtAGCAAGTTTCAAAGTTTTGGGCCAGTTTTCac |
| | | atcatagt(SEQ ID NO: 160) |
| DDX58 | DDX58_CDS_8 | aagtgactgtTGTATGTCAGAAGTTTCCATCTTATCCTCAaca |
| | | tcatagt(SEQ ID NO: 161) |
| DDX58 | DDX58_CDS_9 | aagtgactgtCACTTCTGAAGGTGGACATGAATTCTCACTac |
| | | atcatagt(SEQ ID NO: 162) |
| DDX58 | DDX58_CDS_12 | aagtgactgtACAGATTTCTGCTGTTCATACACTGGGATCac |
| | | atcatagt(SEQ ID NO: 163) |
| DDX58 | DDX58_CDS_13 | aagtgactgtCTGTAACTCTATACCCATGTCTTTCAAAGTaca |
| | | tcatagt(SEQ ID NO: 164) |
| DDX58 | DDX58_CDS_15 | aagtgactgtATGATGTCATTGTTCTCAACAATCTGTTCCaca |
| | | tcatagt(SEQ ID NO: 165) |
| DDX58 | DDX58_CDS_16 | aagtgactgtATCGTTCCCTTTTTAAGGTTGTTCACAAGAaca |
| | | tcatagt(SEQ ID NO: 166) |
| DDX58 | DDX58_CDS_17 | aagtgactgtTGTTGTTTACTAGTGTTGTGGCATTCATCAaca |
| | | tcatagt(SEQ ID NO: 167) |
| DDX58 | DDX58_CDS_18 | aagtgactgtTAATCCAAGGCTTCATCTGTGTTTTTGGCAaca |
| | | tcatagt(SEQ ID NO: 168) |
| DDX58 | DDX58_CDS_21 | aagtgactgtCCGTGATTCCACTTTCCTGAAAAACTTCTGac |
| | | atcatagt(SEQ ID NO: 169) |
| DDX58 | DDX58_CDS_22 | aagtgactgtGCTGAGCTATGATGTATTTAAATTTGTCGCac |
| | | atcatagt(SEQ ID NO: 170) |
| DDX58 | DDX58_CDS_23 | aagtgactgtAATTTGAGATAAGTTTTCGAGGTCTTTGCAac |
| | | atcatagt(SEQ ID NO: 171) |
| DDX58 | DDX58_CDS_24 | aagtgactgtTGTTCATATTTCTGTGTTCCAAATTCCCTAacat |
| | | catagt(SEQ ID NO: 172) |
| DDX58 | DDX58_CDS_26 | aagtgactgtCTTTACAAATCCTGCTCTCTTCATCTTTGTacat |
| | | catagt(SEQ ID NO: 173) |
| DDX58 | DDX58_CDS_27 | aagtgactgtGGGCATCATTATATTTCCGCAAATGTGAAGac |
| | | atcatagt(SEQ ID NO: 174) |
| DDX58 | DDX58_CDS_28 | aagtgactgtCATCTTTCATTCGTGCATGCTCACTGATAAaca |
| | | tcatagt(SEQ ID NO: 175) |
| DDX58 | DDX58_CDS_29 | aagtgactgtAAGATCTTGCTCAATCTCATCGAATCCTGCac |
| | | atcatagt(SEQ ID NO: 176) |
| DDX58 | DDX58_CDS_30 | aagtgactgtTTTCTAGTTCCTGCAGCTTTTCTTCAAATCacat |
| | | catagt(SEQ ID NO: 177) |
| DDX58 | DDX58_CDS_31 | aagtgactgtGAGGTCTTCAAGTTTAGGATTCTCATTGCTac |
| | | atcatagt(SEQ ID NO: 178) |
| DDX58 | DDX58_CDS_33 | aagtgactgtTCCACAAGTGCTCTGGTTTTCACAAAGAGAac |
| | | atcatagt(SEQ ID NO: 179) |
| DDX58 | DDX58_CDS_34 | aagtgactgtACGTCCAGTCAATATGCCAGGTTTTAGAAAac |
| | | atcatagt(SEQ ID NO: 180) |
| DDX58 | DDX58_CDS_38 | aagtgactgtTCATTTTGATGACATTGCCCACATACTCATaca |
| | | tcatagt(SEQ ID NO: 181) |
| DDX58 | DDX58_CDS_39 | aagtgactgtTTCGTCCCATGTCTGAAGGCGTAAAATAGAac |
| | | atcatagt(SEQ ID NO: 182) |
| DDX58 | DDX58_CDS_40 | aagtgactgtATGAGTCTGTATATGCAGAATCTTTTCCCTaca |
| | | tcatagt(SEQ ID NO: 183) |
| DDX58 | DDX58_CDS_41 | aagtgactgtGGTACAGGTTTTGGTTTTTCTTGACTATCTaca |
| | | tcatagt(SEQ ID NO: 184) |
| DDX58 | DDX58_CDS_43 | aagtgactgtTAATGGCATTCCTCTATCACTCTTACGTCAaca |
| | | tcatagt(SEQ ID NO: 185) |
| DDX58 | DDX58_CDS_46 | aagtgactgtTCGGGCACAGAATATCTTTGCTCTTTTTTCaca |
| | | tcatagt(SEQ ID NO: 186) |
| DDX58 | DDX58_CDS_47 | aagtgactgtTCAAATGTCTTGTACTTCACATGGATTCCCaca |
| | | tcatagt(SEQ ID NO: 187) |
| DDX58 | DDX58_CDS_48 | aagtgactgtGAGTACAGTGTCTGAACTCCAGTTGCAATAac |
| | | atcatagt(SEQ ID NO: 188) |
| MITF | MITF_CDS_2 | aagtgactgtTCTTCAGCGGTTGACTTTTGAGTTCGTAATaca |
| | | tcatagt(SEQ ID NO: 189) |
| MITF | MITF_CDS_3 | aagtgactgtATACTGGAGGAGCTTATCGGAGGCTTGGAGa |
| | | catcatagt(SEQ ID NO: 190) |
| MITF | MITF_CDS_4 | aagtgactgtATGAGTTGCTGGCGTAGCAAGATGCGTGATa |
| | | catcatagt(SEQ ID NO: 191) |
| MITF | MITF_CDS_5 | aagtgactgtACTGACGTTTATGGCTGGTGTCTGACTCACac |
| | | atcatagt(SEQ ID NO: 192) |
| MITF | MITF_CDS_7 | aagtgactgtCGTTGGGCTTGCTGTATGTGGTACTTGGTGac |
| | | atcatagt(SEQ ID NO: 193) |
| MITF | MITF_CDS_8 | aagtgactgtAAGTGGTAGAAAGGTACTGCTTTACCTGCTac |
| | | atcatagt(SEQ ID NO: 194) |
| MITF | MITF_CDS_9 | aagtgactgtAAGCTCAGGACTTGGTTGGCATGTTTATTTac |
| | | atcatagt(SEQ ID NO: 195) |
| MITF | MITF_CDS_12 | aagtgactgtATACAGGACGCTCGTGAATGTGTGTTCATGac |
| | | atcatagt(SEQ ID NO: 196) |
| MITF | MITF_CDS_13 | aagtgactgtGGCTAATGATGTCATCGATTACATCATCCAac |
| | | atcatagt(SEQ ID NO: 197) |
| MITF | MITF_CDS_14 | aagtgactgtCCATCAAGCCCAAGATTTCCTCATTATAACac |
| | | atcatagt(SEQ ID NO: 198) |
| MITF | MITF_CDS_15 | aagtgactgtGAGACAGGCAACGTATTTGCCATTTGCAAAa |
| | | catcatagt(SEQ ID NO: 199) |
| MITF | MITF_CDS_16 | aagtgactgtGGCAGACCTTGGTTTCCATAAAGATCAATCac |
| | | atcatagt(SEQ ID NO: 200) |
| MITF | MITF_CDS_17 | aagtgactgtTCAGACTCTGTGAGCTCCCTTTTTATGTTGaca |
| | | tcatagt(SEQ ID NO: 201) |
| MITF | MITF_CDS_18 | aagtgactgtTCTTCTTCGTTCAATCAGGTTGTGATTGTCaca |
| | | tcatagt(SEQ ID NO: 202) |
| MITF | MITF_CDS_19 | aagtgactgtACCTAGTTCTTTAATGCGGTCATTTATGTTaca |
| | | tcatagt(SEQ ID NO: 203) |
| MITF | MITF_CDS_20 | aagtgactgtGCATGTCTGGATCATTTGACTTGGGAATCAac |
| | | atcatagt(SEQ ID NO: 204) |
| MITF | MITF_CDS_21 | aagtgactgtACGGATGCTTTTAAGATGGTTCCCTTGTTCaca |
| | | tcatagt(SEQ ID NO: 205) |
| MITF | MITF_CDS_22 | aagtgactgtTGCTGTTCTCGTTGCAACTTTCGGATATAGaca |
| | | tcatagt(SEQ ID NO: 206) |
| MITF | MITF_CDS_23 | aagtgactgtCAGTTTCTTCTGTCGGTTTTCAAGTTCTTTacat |
| | | catagt(SEQ ID NO: 207) |
| MITF | MITF_CDS_24 | aagtgactgtATGAGCTCGAGCCTGCATTTCAAGTTCCTGac |
| | | atcatagt(SEQ ID NO: 208) |
| MITF | MITF_CDS_26 | aagtgactgtGCTTGATGATCCGATTCACCAAATCTGGAGac |
| | | atcatagt(SEQ ID NO: 209) |
| MITF | MITF_CDS_33 | aagtgactgtAGTGGATCAGTGACACCGACGGGAGAAAGG |
| | | acatcatagt(SEQ ID NO: 210) |
| MITF | MITF_3UTR_10 | aagtgactgtATTAGACTTTATATTTCAGAGCCTTGGTCTaca |
| | | tcatagt(SEQ ID NO: 211) |
| MITF | MITF_3UTR_11 | aagtgactgtCCAATTAGAAATATGCCCAGTTCGGTTTTCac |
| | | atcatagt(SEQ ID NO: 212) |
| MITF | MITF_3UTR_12 | aagtgactgtGGGTGTACAAAGAATGCGGAAAACCACCAG |
| | | acatcatagt(SEQ ID NO: 213) |
| MITF | MITF_3UTR_13 | aagtgactgtTCGCAAAGCTGTCTTTTAAATGCAAGACCTac |
| | | atcatagt(SEQ ID NO: 214) |
| MITF | MITF_3UTR_14 | aagtgactgtGTGTCCAAAAACAGTTCTGAGTGAGACTGTa |
| | | catcatagt(SEQ ID NO: 215) |
| MITF | MITF_3UTR_16 | aagtgactgtATCTCCAAGAATATTGCTATTTTCATGTCCaca |
| | | tcatagt(SEQ ID NO: 216) |
| MITF | MITF_3UTR_17 | aagtgactgtCCCCAATTTAAATGACCATAATGGGCGTATac |
| | | atcatagt(SEQ ID NO: 217) |
| MITF | MITF_3UTR_18 | aagtgactgtAACATTGTTATCAAGTAACAGTAACTTCCCac |
| | | atcatagt(SEQ ID NO: 218) |
| MITF | MITF_3UTR_19 | aagtgactgtGCACTGAAACTAACTTTATAACATTGCTTCac |
| | | atcatagt(SEQ ID NO: 219) |
| MITF | MITF_3UTR_20 | aagtgactgtACTACTGTTATTGCATAGGACTACACAAGTac |
| | | atcatagt(SEQ ID NO: 220) |
| UBC | UBC_CDS_1 | aagtgactgtCTTACCAGTCAGAGTCTTCACGAAGATCTGac |
| | | atcatagt(SEQ ID NO: 221) |
| UBC | UBC_CDS_6 | aagtgactgtCACGAAGATTTGCATCCCACCTCTGAGACGac |
| | | atcatagt(SEQ ID NO: 222) |
| UBC | UBC_CDS_7 | aagtgactgtTCAAGGGTGATGGTCTTGCCAGTGAGTGTCac |
| | | atcatagt(SEQ ID NO: 223) |
| UBC | UBC_CDS_8 | aagtgactgtTGGATCTTTGCTTTGACGTTCTCGATGGTGaca |
| | | tcatagt(SEQ ID NO: 224) |
| UBC | UBC_CDS_9 | aagtgactgtAAAGATCAACCTCTGCTGGTCAGGAGGAATa |
| | | catcatagt(SEQ ID NO: 225) |
| UBC | UBC_CDS_10 | aagtgactgtGTAGACTCTTTCTGGATGTTGTAGTCAGACac |
| | | atcatagt(SEQ ID NO: 226) |
| UBC | UBC_CDS_13 | aagtgactgtCTGATCAGGAGGAATGCCTTCCTTATCTTGac |
| | | atcatagt(SEQ ID NO: 227) |
| UBC | UBC_CDS_15 | aagtgactgtTTTCTGGATGTTGTAGTCAGACAGGGTACGac |
| | | atcatagt(SEQ ID NO: 228) |
| UBC | UBC_CDS_16 | aagtgactgtCTGAGACGGAGTACCAGGTGCAAGGTGGACa |
| | | catcatagt(SEQ ID NO: 229) |
| UBC | UBC_CDS_17 | aagtgactgtCTTGCCAGTGAGTGTCTTCACGAAGATTTGac |
| | | atcatagt(SEQ ID NO: 230) |
| UBC | UBC_CDS_19 | aagtgactgtCTTCCTTGTCTTGGATCTTTGCTTTGACGTacat |
| | | catagt(SEQ ID NO: 231) |
| UBC | UBC_CDS_20 | aagtgactgtAAAGATCAACCTCTGCTGGTCAGGAGGAATa |
| | | catcatagt(SEQ ID NO: 232) |
| UBC | UBC_CDS_21 | aagtgactgtGTAGACTCTTTCTGGATGTTGTAGTCAGACac |
| | | atcatagt(SEQ ID NO: 233) |
| UBC | UBC_CDS_22 | aagtgactgtTTCACGAAGATCTGCATCCCACCTCTGAGAac |
| | | atcatagt(SEQ ID NO: 234) |
| UBC | UBC_CDS_23 | aagtgactgtCACTTCGAGAGTGATGGTCTTACCAGTCAGac |
| | | atcatagt(SEQ ID NO: 235) |
| UBC | UBC_CDS_24 | aagtgactgtTCTTGGATCTTTGCCTTGACATTCTCAATGaca |
| | | tcatagt(SEQ ID NO: 236) |
| UBC | UBC_CDS_26 | aagtgactgtACAGGGTACGACCATCTTCCAGCTGTTTTCac |
| | | atcatagt(SEQ ID NO: 237) |
| UBC | UBC_CDS_27 | aagtgactgtCAAGGTGGACTCTTTCTGGATGTTGTAGTCac |
| | | atcatagt(SEQ ID NO: 238) |
| UBC | UBC_CDS_28 | aagtgactgtTTCACGAAGATCTGCATCCCACCTCTGAGAac |
| | | atcatagt(SEQ ID NO: 239) |
| UBC | UBC_CDS_29 | aagtgactgtCACCTCGAGAGTGATGGTCTTACCAGTCAGac |
| | | atcatagt(SEQ ID NO: 240) |
| UBC | UBC_CDS_30 | aagtgactgtTCTTGGATCTTTGCCTTGACATTCTCAATGaca |
| | | tcatagt(SEQ ID NO: 241) |
| UBC | UBC_CDS_33 | aagtgactgtTTCACGAAGATCTGCATCCCACCTCTAAGAac |
| | | atcatagt(SEQ ID NO: 242) |
| UBC | UBC_CDS_34 | aagtgactgtCACTTCGAGAGTGATGGTCTTACCAGTCAGac |
| | | atcatagt(SEQ ID NO: 243) |
| UBC | UBC_CDS_35 | aagtgactgtTCTTGGATCTTTGCCTTGACATTCTCAATGaca |
| | | tcatagt(SEQ ID NO: 244) |
| UBC | UBC_CDS_38 | aagtgactgtTTCACGAAGATCTGCATCCCACCTCTAAGAac |
| | | atcatagt(SEQ ID NO: 245) |
| UBC | UBC_CDS_39 | aagtgactgtCACTTCGAGAGTGATGGTCTTACCAGTCAGac |
| | | atcatagt(SEQ ID NO: 246) |
| UBC | UBC_CDS_40 | aagtgactgtTCTTGGATCTTTGCCTTGACATTCTCAATGaca |
| | | tcatagt(SEQ ID NO: 247) |
| UBC | UBC_CDS_43 | aagtgactgtCTTACCAGTCAGGGTCTTCACGAAGATTTGac |
| | | atcatagt(SEQ ID NO: 248) |
| UBC | UBC_CDS_45 | aagtgactgtCTGATCAGGAGGGATGCCTTCCTTATCTTGac |
| | | atcatagt(SEQ ID NO: 249) |
| UBC | UBC_CDS_46 | aagtgactgtATCTTCCAGCTGTTTCCCAGCAAAGATCAAac |
| | | atcatagt(SEQ ID NO: 250) |
| UBC | UBC_CDS_48 | aagtgactgtCTCAAGCGCAGGACCAAGTGCAGAGTGGACa |
| | | catcatagt(SEQ ID NO: 251) |
| UBC | UBC_5UTR_1 | aagtgactgtATCCACAAACAAGAACTGCGACCCAAATCCa |
| | | catcatagt(SEQ ID NO: 252) |
| MMP1 | MMP1_CDS_1 | aagtgactgtGGCAAATCTGGCGTGTAATTTTCAATCCTGac |
| | | atcatagt(SEQ ID NO: 253) |
| MMP1 | MMP1_CDS_3 | aagtgactgtCCTTGGTGAATGTCAGAGGTGTGACATTACac |
| | | atcatagt(SEQ ID NO: 254) |
| MMP1 | MMP1_CDS_4 | aagtgactgtAAAGATATCATGATGTCTGCTTGACCCTCAac |
| | | atcatagt(SEQ ID NO: 255) |
| MMP1 | MMP1_CDS_5 | aagtgactgtAAAGGAGAGTTGTCCCGATGATCTCCCCTGac |
| | | atcatagt(SEQ ID NO: 256) |
| MMP1 | MMP1_CDS_7 | aagtgactgtAGTTGTACTCTCTGAAATTGTTGGTCCACCac |
| | | atcatagt(SEQ ID NO: 257) |
| MMP1 | MMP1_CDS_9 | aagtgactgtCAAAGCCCCGATATCAGTAGAATGGGAGAGa |
| | | catcatagt(SEQ ID NO: 258) |
| MMP1 | MMP1_CDS_10 | aagtgactgtAACATCACCACTGAAGGTGTAGCTAGGGTAa |
| | | catcatagt(SEQ ID NO: 259) |
| MMP1 | MMP1_CDS_11 | aagtgactgtTTGGATGCCATCAATGTCATCCTGAGCTAGac |
| | | atcatagt(SEQ ID NO: 260) |
| MMP1 | MMP1_CDS_12 | aagtgactgtCTGGACAGGATTTTGGGAACGTCCATATATac |
| | | atcatagt(SEQ ID NO: 261) |
| MMP 1 | MMP1_CDS_13 | aagtgactgtAAAGGTTAGCTTACTGTCACACGCTTTTGGac |
| | | atcatagt(SEQ ID NO: 262) |
| MMP1 | MMP1_CDS_14 | aagtgactgtAAAGAACATCACTTCTCCCCGAATCGTAGTac |
| | | atcatagt(SEQ ID NO: 263) |
| MMP1 | MMP1_CDS_15 | aagtgactgtGAAGGGATTTGTGCGCATGTAGAATCTGTCac |
| | | atcatagt(SEQ ID NO: 264) |
| MMP1 | MMP1_CDS_16 | aagtgactgtGGACTTCATCTCTGTCGGCAAATTCGTAAGac |
| | | atcatagt(SEQ ID NO: 265) |
| MMP1 | MMP1_CDS_17 | aagtgactgtCACATTCTGTCCCTGAACAGCCCAGTACTTac |
| | | atcatagt(SEQ ID NO: 266) |
| MMP1 | MMP1_CDS_18 | aagtgactgtCAAAGGAGCTGTAGATGTCCTTGGGGTATCa |
| | | catcatagt(SEQ ID NO: 267) |
| MMP1 | MMP1_CDS_19 | aagtgactgtAGCATCGATATGCTTCACAGTTCTAGGGAAac |
| | | atcatagt(SEQ ID NO: 268) |
| MMP1 | MMP1_CDS_20 | aagtgactgtCATCATACCTCCAGTATTTGTTAGCAACAAac |
| | | atcatagt(SEQ ID NO: 269) |
| MMP1 | MMP1_CDS_21 | aagtgactgtGGGATAACCTGGATCCATAGATCGTTTATAac |
| | | atcatagt(SEQ ID NO: 270) |
| MMP1 | MMP1_CDS_22 | aagtgactgtCCAATTCCAGGAAAGTCATGTGCTATCATTac |
| | | atcatagt(SEQ ID NO: 271) |
| MMP1 | MMP1_CDS_23 | aagtgactgtCTGGAGAGTCAAAATTCTCTTCGTTTTAGGac |
| | | atcatagt(SEQ ID NO: 272) |
| MMP1 | MMP1_CDS_24 | aagtgactgtCAATTTTTCCTGCAGTTGAACCAGCTATTAac |
| | | atcatagt(SEQ ID NO: 273) |
| MMP1 | MMP1_3UTR_1 | aagtgactgtAAATAGACAGTTCTTCAGGAAAACACCTTCa |
| | | catcatagt(SEQ ID NO: 274) |
| MMP1 | MMP1_3UTR_2 | aagtgactgtATATTCTGTGTATCAGTGACTCTAGAGGTTac |
| | | atcatagt(SEQ ID NO: 275) |
| MMP1 | MMP1_3UTR_3 | aagtgactgtACTTTTTGTACCCACCATTIGTGGAACTAAac |
| | | atcatagt(SEQ ID NO: 276) |
| NGFR | NGFR_CDS_1 | aagtgactgtCAGTCGTCTCATCCTGGTAGTAGCCGTAGGac |
| | | atcatagt(SEQ ID NO: 277) |
| NGFR | NGFR_CDS_2 | aagtgactgtGTTCTGCTTGTCCTGGCAGGAGAACACGAGa |
| | | catcatagt(SEQ ID NO: 278) |
| NGFR | NGFR_CDS_3 | aagtgactgtTCCACGTGGTTGGCCTCGTCGGAATACGTGac |
| | | atcatagt(SEQ ID NO: 279) |
| NGFR | NGFR_CDS_4 | aagtgactgtCTCTGGGGGTGTGGACCGTGTAATCCAACGa |
| | | catcatagt(SEQ ID NO: 280) |
| NGFR | NGFR_CDS_5 | aagtgactgtCCGTGCTGGCTATGAGGTCTTGTTCTGGAGac |
| | | atcatagt(SEQ ID NO: 281) |
| NGFR | NGFR_CDS_6 | aagtgactgtCAATAGACAGGGATGAGGTTGTCGGTGGTGa |
| | | catcatagt(SEQ ID NO: 282) |
| NGFR | NGFR_CDS_7 | aagtgactgtTTGCAGCTGTTCCACCTCTTGAAGGCTATGac |
| | | atcatagt(SEQ ID NO: 283) |
| NGFR | NGFR_CDS_8 | aagtgactgtCTCATGGGTAAAGGAGTCTATGTGCTCGGGa |
| | | catcatagt(SEQ ID NO: 284) |
| NGFR | NGFR_CDS_9 | aagtgactgtAGACTCTCCACGAGGTCGGCTCGCTGGATGa |
| | | catcatagt(SEQ ID NO: 285) |
| NGFR | NGFR_CDS_10 | aagtgactgtCACACCGGGGATGTGGCAGTGGACTCACTGa |
| | | catcatagt(SEQ ID NO: 286) |
| NGFR | NGFR_3UTR_1 | aagtgactgtGGGGTTGGCTGGAGCATCGGTTGTCGGAATa |
| | | catcatagt(SEQ ID NO: 287) |
| NGFR | NGFR_3UTR_2 | aagtgactgtTGGACAGGAAGTGTGGTCAGAAGTGAAGGG |
| | | acatcatagt(SEQ ID NO: 288) |
| NGFR | NGFR_3UTR_3 | aagtgactgtCAAGTTCACAGTGTAGAGTCCATGGCTCTGac |
| | | atcatagt(SEQ ID NO: 289) |
| NGFR | NGFR_3UTR_5 | aagtgactgtCTTCAAGCCATTTCCCAGAAGGTCACCTGCac |
| | | atcatagt(SEQ ID NO: 290) |
| NGFR | NGFR_3UTR_6 | aagtgactgtTGGAGACAGCGTGGAAGGCAAAGCTGACTTa |
| | | catcatagt(SEQ ID NO: 291) |
| NGFR | NGFR_3UTR_7 | aagtgactgtACGAACCCCAAACCTGACTCCATCATGGACa |
| | | catcatagt(SEQ ID NO: 292) |
| NGFR | NGFR_3UTR_9 | aagtgactgtAGACTGGAGCCATGCCACTGATGTCACACAa |
| | | catcatagt(SEQ ID NO: 293) |
| NGFR | NGFR_3UTR_10 | aagtgactgtTGTTAGCTCCGGGTCCATGTCGGGATGGAGac |
| | | atcatagt(SEQ ID NO: 294) |
| NGFR | NGFR_3UTR_11 | aagtgactgtGAGTGAAGACGGCCGAGCAGACCTCAATTCa |
| | | catcatagt(SEQ ID NO: 295) |
| NGFR | NGFR_3UTR_12 | aagtgactgtATTCCTCCACGTGTAAGCCCTTGAAGACAGac |
| | | atcatagt(SEQ ID NO: 296) |
| NGFR | NGFR_3UTR_13 | aagtgactgtTGGCCCACTTTTCTTCATCACAACCTTCTGaca |
| | | tcatagt(SEQ ID NO: 297) |
| NGFR | NGFR_3UTR_14 | aagtgactgtAGTCGAAGTCAATTCCTTCTTGCCGCATTCac |
| | | atcatagt(SEQ ID NO: 298) |
| NGFR | NGFR_3UTR_16 | aagtgactgtCAAAGAATTGCCATTTACTACAGTGCTCCTac |
| | | atcatagt(SEQ ID NO: 299) |
| NGFR | NGFR_3UTR_17 | aagtgactgtGTGAGTTTCCTCCCCTCATCACAGGTTGAGac |
| | | atcatagt(SEQ ID NO: 300) |
| GFP | Invertedbackbone_ | aagtgactgtTTACGTCGCCGTCCAGCTCGACCAGGATGGac |
| | GFP1 | atcatagt(SEQ ID NO: 301) |
| GFP | Invertedbackbone_ | aagtgactgtTAGGTGGCATCGCCCTCGCCCTCGCCGGACac |
| | GFP2 | atcatagt(SEQ ID NO: 302) |
| GFP | Invertedbackbone_ | aagtgactgtGTGGTGCAGATGAACTTCAGGGTCAGCTTGa |
| | GFP3 | catcatagt(SEQ ID NO: 303) |
| GFP | Invertedbackbone_ | aagtgactgtATGTGGTCGGGGTAGCGGCTGAAGCACTGCa |
| | GFP4 | catcatagt(SEQ ID NO: 304) |
| GFP | Invertedbackbone_ | aagtgactgtGCGCTCCTGGACGTAGCCTTCGGGCATGGCac |
| | GFP5 | atcatagt(SEQ ID NO: 305) |
| GFP | Invertedbackbone_ | aagtgactgtCCCTCGAACTTCACCTCGGCGCGGGTCTTGac |
| | GFP6 | atcatagt(SEQ ID NO: 306) |
| GFP | Invertedbackbone_ | aagtgactgtAGTTGTACTCCAGCTTGTGCCCCAGGATGTac |
| | GFP7 | atcatagt(SEQ ID NO: 307) |
| GFP | Invertedbackbone_ | aagtgactgtGCTGCCGTCCTCGATGTTGTGGCGGATCTTac |
| | GFP8 | atcatagt(SEQ ID NO: 308) |
| GFP | Invertedbackbone_ | aagtgactgtTTGTCGGGCAGCAGCACGGGGCCGTCGCCGa |
| | GFP9 | catcatagt(SEQ ID NO: 309) |
| GFP | Invertedbackbone_ | aagtgactgtACTCCAGCAGGACCATGTGATCGCGCTTCTac |
| | GFP10 | atcatagt(SEQ ID NO: 310) |

SEQ ID NOS 301-310 were ordered with 5′ phosphate (/5Phos/) modification.

clampFISH 2.0 Amplifier Probes Design and Construction

clampFISH 2.0 amplifier probes (secondary probes and tertiary probes) were constructed as follows. To design amplifier probe sets 1 and 2, two 30mer ‘landing pad’ sequences (one for the secondary, one for the tertiary) were manually generated with approximately 50% GC content and “AT” at the center, and the 30mer was then concatenated to itself to form a 60mer backbone sequence. 15mer arms were added on each end of the 60mer secondary backbones, such that arms were reverse complements to their paired tertiary backbone, and similarly added 15mer arms to each tertiary backbone to be reverse complements to their paired secondary backbone, thus completing each amplifier probe's full 90mer probe sequence. For the remaining amplifier series, 500,000 random 30mers were generated, the middle two bases were replaced with “AT”. Sequences where the percent GC content of the left 15 nucleotides and the right 15 nucleotides were both between 45% and 55% were kept, and then the remaining two 30mers were concatenated together to create a 60mer backbone sequence. Backbone sequences with stretches of 3 or more C, 3 or more G, or 5 or more G or C bases were discarded. For amplifier series 3 to 7, selected were the backbones where the free energy of each backbone's folded structure was greater than-2 kcal/mol as predicted using the DINAMelt web server (Markham and Zuker 2005), selected those without hits against the human transcriptome using BLAST (NCBI) added two 15mer arms to each backbone as before to generate a 90mer amplifier probes, and then selected the five 90mer amplifier probe pairs where the free energy of folding was the least negative as predicted using DINAMelt. For amplifier series 8 to 15, the same steps were followed to generate 60mer backbones (using a different random number generator seed), and then NUPACK was used to predict the minimum free energy of its folded structure, accepting those with a value greater than-1.5 kcal/mol. Half the 60mer sequences were designated to be secondary backbones and the other half to be tertiary backbones and then each secondary backbone was paired with a tertiary backbone. NUPACK was again used to keep only those with a minimum free energy greater than-2.0 kcal/mol. Off-target binding was checked for against the human transcriptome using BLAST, both using a spliced transcriptome database and a custom-generated transcriptome database with unspliced transcripts, NUPACK was used to keep only those with strong off-target binding to RNAs, the sum of the RNA transcripts' maximum FPKM from the ENCODE RNA-seq datasets was taken to generate an off-target FPKM for each secondary and tertiary probe. Secondary and tertiary probe pairs were chosen where each probes' FPKM sum is ≤500 when using the spliced transcript database and ≤2500 when using the unspliced transcript database. Any amplifier sets with probes hitting genomic repeats were then dropped using repeatmasker (repeatmasker.org). NUPACK was used to simulate binding against other probes of the same probe type (each secondary against other secondaries, each tertiary against other tertiaries), and 4 amplifier sets where the predicted binding energy to another probe was <−23 kcal/mol were discarded.

Amplifier probes were ordered from IDT as 89mers with a 5′ hexynyl modification for 15 amplifier sets in total (15 secondaries and 15 tertiaries). In separate reactions for each amplifier probe, an azido-dATP (N6-(6-Azido) hexyl-3′-dATP, Jena Bioscience, NU-1707L) was added to the probes' 3′ ends with Terminal Transferase, thus completing the 90mer amplifier sequence. Ammonium acetate was then added to 2.5M and magnesium chloride to 10 mM, and then centrifugation was performed twice at 17,000 g where each time all but the bottom 10 μL of solution was pipetted to a new tube. The probes were ethanol precipitated, resuspended in 200 μL nuclease-free water, centrifuged in the tube at 17,000 g and all but the bottom 20 μL was pipetted into a new tube.

The following oligonucleotides shown in Table 2 were ordered from IDT dry and then resuspended to 400 μM in nuclease-free water. Standard purification (not HPLC) was used for all of the below oligos, including those with 5′ hexynyl modifications

TABLE 2

| Relevant | | |
|---|---|---|
| amplifier | Oligo name/ Oligo name, | |
| set | as ordered | Oligo sequence |
| all | Primary_adapter | ACAGTCACTTCAACACTCAG (SEQ ID NO: 311) |
| all | circularizer_oligo/ | TCTTGACGAGAATGTCTtACTATGATG (SEQ ID |
| | Bridge5_R5_27 mer | NO: 312) |
| 1 | Pr1_RightArm_5Hex | agacattctcgtcaagatTCACGCTTATTCGCATGTTTAG |
| | | TACGCCCTctgagtgttg (SEQ ID NO: 313) |
| 2 | Pr2_RightArm_5Hex | agacattctcgtcaagatACTTATACTCGCCGATAAAAA |
| | | CGTCGCACGctgagtgttg (SEQ ID NO: 314) |
| 3 | Pr3_RightArm_5Hex/ | agacattctcgtcaagatGTGTTTTGCGTCCGATTGTACC |
| | Pr3_RightArm | GATACCACctgagtgttg (SEQ ID NO: 315) |
| 4 | Pr4_RightArm_5Hex/ | agacattctcgtcaagatATACCTACACCGACATAGACG |
| | Pr4_RightArm | TAAGCCAGCctgagtgttg (SEQ ID NO: 316) |
| 5 | Pr5_RightArm_5Hex/ | agacattctcgtcaagatAGTCGGTGTCTTAGATCGGTT |
| | Pr5_RightArm | CGCTCTTTActgagtgttg (SEQ ID NO: 317) |
| 6 | Pr6_RightArm_5Hex/ | agacattctcgtcaagatGCCATTGGTATGCTATTAACC |
| | Pr6_RightArm | GCTCGACTActgagtgttg (SEQ ID NO: 318) |
| 7 | Pr7_RightArm_5Hex/ | agacattctcgtcaagatTAGAACAACGGTCGATCCACT |
| | Pr7_RightArm | TCGCTCACActgagtgttg (SEQ ID NO: 319) |
| 8 | Pr8_RightArm_5Hex | agacattctcgtcaagatCTCTGTACGGTTCTATCAGATT |
| | | GTCGAGTCctgagtgttg (SEQ ID NO: 320) |
| 9 | Pr9_RightArm_5Hex | agacattctcgtcaagatTACGGTGGTGAGAAATTGGTG |
| | | TCTGATGCGctgagtgttg (SEQ ID NO: 321) |
| 10 | Pr10_RightArm_5Hex | agacattctcgtcaagatGTGTGTATTCCGCAATCCGCT |
| | | GATACTTGTctgagtgttg (SEQ ID NO: 322) |
| 11 | Pr11_RightArm_5Hex | agacattctcgtcaagatTGTGGTTCTGCAAGATGTAGG |
| | | TAGTGGTACctgagtgttg (SEQ ID NO: 323) |
| 12 | Pr12_RightArm_5Hex | agacattctcgtcaagatGGTGTAAGAGTCCGATGCGTT |
| | | GTTTGTCCTctgagtgttg (SEQ ID NO: 324) |
| 13 | Pr13_RightArm_5Hex | agacattctcgtcaagatGACAATAACCGACGATAACTC |
| | | CGATACGCTctgagtgttg (SEQ ID NO: 325) |
| 14 | Pr14_RightArm_5Hex | agacattctcgtcaagatTGAATGGCTCGACTATGGTGT |
| | | GTTCGTTTGctgagtgttg (SEQ ID NO: 326) |
| 15 | Pr15_RightArm_5Hex | agacattctcgtcaagatGTTCGGTTGACACTATCACGC |
| | | TTTGAATCCctgagtgttg (SEQ ID NO: 327) |
| 1 | S1_89nt_5Hex | tgcgaataagcgtgaAACTAACGTCGGGAATTCGTCG |
| | | TGACTAGCAACTAACGTCGGGAATTCGTCGTG |
| | | ACTAGCagggcgtactaaac (SEQ ID NO: 328) |
| 2 | S2_89nt_5Hex | tcggcgagtataagtAGACGGTCGAGTATATGGCGAA |
| | | TCGTTAAGAGACGGTCGAGTATATGGCGAATC |
| | | GTTAAGcgtgcgacgttttt (SEQ ID NO: 329) |
| 3 | S3_89nt_5Hex | tcggacgcaaaacacACAACTAGGTCGGCATGGACGC |
| | | TATATGAGACAACTAGGTCGGCATGGACGCT |
| | | ATATGAGgtggtatcggtaca (SEQ ID NO: 330) |
| 4 | S4_89nt_5Hex | tgtcggtgtaggtatTCGTCCACTCGTCTATTTGCGGTT |
| | | GCGTGTTCGTCCACTCGTCTATTTGCGGTTGC |
| | | GTGTgctggcttacgtct (SEQ ID NO: 331) |
| 5 | S5_89nt_5Hex | tctaagacaccgactAACCGAACGTACTGATATTACC |
| | | GTCGAGCAAACCGAACGTACTGATATTACCGT |
| | | CGAGCAtaaagagcgaaccg (SEQ ID NO: 332) |
| 6 | S6_89nt_5Hex | tagcataccaatggcCTAGTAGCTTAGCGATACGGTA |
| | | GGTCTAGTCTAGTAGCTTAGCGATACGGTAGG |
| | | TCTAGTtagtcgagcggtta (SEQ ID NO: 333) |
| 7 | S7_89nt_5Hex | tcgaccgttgttctaCCTTAACTAGAGCGATACGAGCC |
| | | TACCGAACCTTAACTAGAGCGATACGAGCCTA |
| | | CCGAAtgtgagcgaagtgg (SEQ ID NO: 334) |
| 8 | S8_89nt_5Hex | tagaaccgtacagagTGACCTCAACCGTTATCCAGCG |
| | | AAACAAGATGACCTCAACCGTTATCCAGCGA |
| | | AACAAGAgactcgacaatctg (SEQ ID NO: 335) |
| 9 | S9_89nt_5Hex | tttctcaccaccgtaTGCTTCTATCCGTGATACCGACTT |
| | | CGTTAGTGCTTCTATCCGTGATACCGACTTCG |
| | | TTAGcgcatcagacacca (SEQ ID NO: 336) |
| 10 | S10_89nt_5Hex | ttgcggaatacacacCTAACAAGCGACTCATTGGTTCC |
| | | AGATGCACTAACAAGCGACTCATTGGTTCCAG |
| | | ATGCAacaagtatcagcgg (SEQ ID NO: 337) |
| 11 | S11_89nt_5Hex | tcttgcagaaccacaCAACTAGGAGCTTCATCACGAC |
| | | CAAAGACTCAACTAGGAGCTTCATCACGACC |
| | | AAAGACTgtaccactacctac (SEQ ID NO: 338) |
| 12 | S12_89nt_5Hex | tcggactcttacaccTAGGTTCACTGCTCATACGCTCG |
| | | AATCTTCTAGGTTCACTGCTCATACGCTCGAA |
| | | TCTTCaggacaaacaacgc (SEQ ID NO: 339) |
| 13 | S13_89nt_5Hex | tcgtcggttattgtcTCGCAAACGGAGATATATGATCG |
| | | GCAACACTCGCAAACGGAGATATATGATCGG |
| | | CAACACagcgtatcggagtt (SEQ ID NO: 340) |
| 14 | S14_89nt_5Hex | tagtogagccattcaTGTAACGTCTGAGGATTCTTGTC |
| | | CGCACAATGTAACGTCTGAGGATTCTTGTCCG |
| | | CACAAcaaacgaacacacc (SEQ ID NO: 341) |
| 15 | S15_89nt_5Hex | tagtgtcaaccgaacCATAGTAGAAGGCGATCTCCAG |
| | | TGACTCATCATAGTAGAAGGCGATCTCCAGTG |
| | | ACTCATggattcaaagcgtg (SEQ ID NO: 342) |
| 1 | T1_89nt_5Hex | ttcccgacgttagttTCACGCTTATTCGCATGTTTAGTA |
| | | CGCCCTTCACGCTTATTCGCATGTTTAGTACG |
| | | CCCTgctagtcacgacga (SEQ ID NO: 343) |
| 2 | T2_89nt_5Hex | tatactcgaccgtctACTTATACTCGCCGATAAAAACG |
| | | TCGCACGACTTATACTCGCCGATAAAAACGTC |
| | | GCACGcttaacgattcgcc (SEQ ID NO: 344) |
| 3 | T3_89nt_5Hex | tgccgacctagttgtGTGTTTTGCGTCCGATTGTACCG |
| | | ATACCACGTGTTTTGCGTCCGATTGTACCGAT |
| | | ACCACctcatatagcgtcc (SEQ ID NO: 345) |
| 4 | T4_89nt_5Hex | tagacgagtggacgaATACCTACACCGACATAGACGT |
| | | AAGCCAGCATACCTACACCGACATAGACGTA |
| | | AGCCAGCacacgcaaccgcaa (SEQ ID NO: 346) |
| 5 | T5_89nt_5Hex | tcagtacgttcggttAGTCGGTGTCTTAGATCGGTTCG |
| | | CTCTTTAAGTCGGTGTCTTAGATCGGTTCGCTC |
| | | TTTAtgctcgacggtaat (SEQ ID NO: 347) |
| 6 | T6_89nt_5Hex | tcgctaagctactagGCCATTGGTATGCTATTAACCGC |
| | | TCGACTAGCCATTGGTATGCTATTAACCGCTC |
| | | GACTAactagacctaccgt (SEQ ID NO: 348) |
| 7 | T7_89nt_5Hex | tcgctctagttaaggTAGAACAACGGTCGATCCACTTC |
| | | GCTCACATAGAACAACGGTCGATCCACTTCGC |
| | | TCACAttcggtaggctcgt (SEQ ID NO: 349) |
| 8 | T8_89nt_5Hex | taacggttgaggtcaCTCTGTACGGTTCTATCAGATTG |
| | | TCGAGTCCTCTGTACGGTTCTATCAGATTGTC |
| | | GAGTCtcttgtttcgctgg (SEQ ID NO: 350) |
| 9 | T9_89nt_5Hex | tcacggatagaagcaTACGGTGGTGAGAAATTGGTGT |
| | | CTGATGCGTACGGTGGTGAGAAATTGGTGTCT |
| | | GATGCGctaacgaagtcggt (SEQ ID NO: 351) |
| 10 | T10_89nt_5Hex | tgagtcgcttgttagGTGTGTATTCCGCAATCCGCTGA |
| | | TACTTGTGTGTGTATTCCGCAATCCGCTGATA |
| | | CTTGTtgcatctggaacca (SEQ ID NO: 352) |
| 11 | T11_89nt_5Hex | tgaagctcctagttgTGTGGTTCTGCAAGATGTAGGTA |
| | | GTGGTACTGTGGTTCTGCAAGATGTAGGTAGT |
| | | GGTACagtctttggtcgtg (SEQ ID NO: 353) |
| 12 | T12_89nt_5Hex | tgagcagtgaacctaGGTGTAAGAGTCCGATGCGTTG |
| | | TTTGTCCTGGTGTAAGAGTCCGATGCGTTGTT |
| | | TGTCCTgaagattcgagcgt (SEQ ID NO: 354) |
| 13 | T13_89nt_5Hex | tatctccgtttgcgaGACAATAACCGACGATAACTCCG |
| | | ATACGCTGACAATAACCGACGATAACTCCGAT |
| | | ACGCTgtgttgccgatcat (SEQ ID NO: 355) |
| 14 | T14_89nt_5Hex | tcctcagacgttacaTGAATGGCTCGACTATGGTGTGT |
| | | TCGTTTGTGAATGGCTCGACTATGGTGTGTTC |
| | | GTTTGttgtgcggacaaga (SEQ ID NO: 356) |
| 15 | T15_89nt_5Hex | tcgccttctactatgGTTCGGTTGACACTATCACGCTTT |
| | | GAATCCGTTCGGTTGACACTATCACGCTTTGA |
| | | ATCCatgagtcactggag (SEQ ID NO: 357) |

SEQ ID NOs: 313-357 are modified with Hexynyl at 5′ end; Synthesis scale: SEQ ID NO: 311-1 umol; SEQ ID NO: 312-25 nmol; and SEQ ID NOS: 313-357-100 nm
SEQ ID NO: 311 is a 20mer ligation adapter; 250 nmol scale is recommended since water barely fits in the tube for 400 μM resuspension concentration. This oligo is identical to the Padlock9_rightadapter from clampFISH 1.0.
SEQ ID NO: 312 is a circularizer oligonucleotide; Lowercase ‘t’ pairs with the azido-dATP that is added to the 3′ end of the RNA-targeting oligonucleotide.
SEQ ID NO: 313-SEQ IS NO: 327 are sequences comprising: Amplifier-specific oligo (58mer) with 5′ Hexynyl, for ligation to RNA-binding oligo.
SEQ ID NO: 328-SEQ ID NO: 342 are sequences comprising: Secondary probe (89mer, before 3′ amino-dATP).
SEQ ID NO: 343-SEQ ID NO: 357 are sequences comprising: Tertiary probe (89mer, before 3′ amino-dATP).
clampFISH 2.0 Readout Probe Design and Construction

For the amplifier screen experiment, a 20 nucleotide readout probe was designed to bind to the center of the 30mer landing pad sequences of each secondary probe, which were ordered from IDT with a 3′ Amino modifier (/3AmMO/), coupled to Atto 647N NHS-ester (ATTO-TEC, AD 647N-31), ethanol precipitated, purified by high-performance liquid chromatography (HPLC) (Raj et al. 2008), and resuspended in TE pH 8.0 buffer (Invitrogen, AM9849).

For all other experiments two readout probes were designed for each amplifier set: one to bind to the secondary probe, and one to bind to the tertiary probe, where each was designed to bind to the center of the probe's 30mer landing pad sequences. Readout probe lengths chosen such that the Gibbs free energy of binding to their target amplifier backbone (DNA: DNA binding) was −22 kcal/mol or −24 kcal/mol, as calculated by MATLAB's oligoprop function (based on the parameters from (Sugimoto et al. 1996)), and then ordered from IDT with a 3′ Amino modifier. The two readout probes targeting a given amplifier set were pooled together and then coupled to one of four NHS-ester dyes (Atto 488, ATTO-TEC, AD 488-31; Cy3, Sigma-Aldrich, GEPA23001; Alexa Fluor 594, ThermoFisher, A20004; or Atto 647N, ATTO-TEC, AD 647N-31), ethanol precipitated, purified by HPLC, and resuspended in TE pH 8.0 buffer, except for readout probes coupled to Atto 488 which were not pooled until after the HPLC steps.

TABLE 3
Readout probes

| Readout | Name of | | Target | | | | | | | |
|---|---|---|---|---|---|---|---|---|---|---|
| probe | readout | | Gibbs | | | | | | | |
| name | set it | Amplifier | Free | | Primary | Probe | Probe Sequence | | Predicted | Gibbs Free |
| once | belongs | probe | Energy | Dye | probsets | length | (without 3′ | % | Gibbs | Energy Target |
| coupled | to | targeted | (kcal/mol) | coupled to | used with | (bases) | amine) | GC | Free Energy | (kcal/mol) |
| S9_22gfe_ | ser9_ | S9 | −22 | Atto 488 | UBC | 14 | TCGGTATCAC | 50 | −22.052935 | −22 |
| Atto488 | 22gfe_ | | | | | | GGAT (SEQ ID | | | |
| | Atto488 | | | | | | NO: 358) | | | |
| T9_22gfe_ | ser9_ | T9 | −22 | Atto 488 | UBC | 16 | AGACACCAA | 37.5 | −22.84086 | −22 |
| Atto488 | 22gfe_ | | | | | | TTTCTCA (SEQ | | | |
| | Atto488 | | | | | | ID NO: 359) | | | |
| S7_24gfe_ | ser7_ | S7 | −24 | Cy3 | WNT5A | 15 | GCTCGTATCG | 53.33 | −23.88645 | −24 |
| Cy3 | 24gfe_ | | | | | | CTCTA (SEQ ID | | | |
| | Cy3 | | | | | | NO: 360) | | | |
| T7_24gfe_ | ser7_ | T7 | −24 | Cy3 | WNT5A | 14 | AGTGGATCG | 57.14 | −23.37829 | −24 |
| Cy3 | 24gfe_ | | | | | | ACCGT (SEQ | | | |
| | Cy3 | | | | | | ID NO: 361) | | | |
| S1_24gfe_ | ser1_ | S1 | −24 | Cy3 | NGFR | 14 | CGACGAATTC | 57.14 | −24.022905 | −24 |
| Cy3 | 24gfe_ | | | | | | CCGA (SEQ ID | | | |
| | Cy3 | | | | | | NO: 362) | | | |
| T1_24gfe_ | ser1_ | T1 | −24 | Cy3 | NGFR | 17 | ACTAAACATG | 29.41 | −23.721385 | −24 |
| Cy3 | 24gfe_ | | | | | | CGAATAA (SE | | | |
| | Cy3 | | | | | | Q ID NO: 363) | | | |
| S10_ | ser10_ | S10 | −24 | Cy3 | ITGA3 | 15 | GAACCAATG | 53.33 | −23.503645 | −24 |
| 24gfe_ | 24gfe_ | | | | | | AGTCGC (SEQ | | | |
| Cy3 | Cy3 | | | | | | ID NO: 364) | | | |
| T10_ | ser10_ | T10 | −24 | Cy3 | ITGA3 | 14 | AGCGGATTGC | 57.14 | −24.392 | −24 |
| 24gfe_ | 24gfe_ | | | | | | GGAA (SEQ ID | | | |
| Cy3 | Cy3 | | | | | | NO: 365) | | | |
| S6_24gfe_ | ser6_ | S6 | −24 | Alexa | DDX58 | 16 | CTACCGTATC | 50 | −24.48493 | −24 |
| Alexa | 24gfe_ | | | Fluor 594 | | | GCTAAG (SEQ | | | |
| 594 | Alex | | | | | | ID NO: 366) | | | |
| | a594 | | | | | | | | | |
| T6_24gfe_ | ser6_ | T6 | −24 | Alexa | DDX58 | 16 | GCGGTTAATA | 43.75 | −23.22345 | −24 |
| Alexa | 24gfe_ | | | Fluor 594 | | | GCATAC (SEQ | | | |
| 594 | Alex | | | | | | ID NO: 367) | | | |
| | a594 | | | | | | | | | |
| S5_22gfe_ | ser5_ | S5 | −22 | Alexa | FN1 | 16 | GGTAATATCA | 37.5 | −21.88308 | −22 |
| Alexa | 22gfe_ | | | Fluor 594 | | | GTACGT (SEQ | | | |
| 594 | Alex | | | | | | ID NO: 368) | | | |
| | a594 | | | | | | | | | |
| T5_22gfe_ | ser5_ | T5 | −22 | Alexa | FN1 | 15 | GAACCGATCT | 46.67 | −21.61496 | −22 |
| Alexa | 22gfe_ | | | Fluor 594 | | | AAGAC (SEQ | | | |
| 594 | Alex | | | | | | ID NO: 369) | | | |
| | a594 | | | | | | | | | |
| S14_ | ser14_ | S14 | −22 | Alexa | MMP1 | 15 | GACAAGAAT | 46.67 | −21.138135 | −22 |
| 22gfe_ | 22gfe_ | | | Fluor 594 | | | CCTCAG (SEQ | | | |
| Alex | Alexa | | | | | | ID NO: 370) | | | |
| a594 | 594 | | | | | | | | | |
| T14_ | ser14_ | T14 | −22 | Alexa | MMP1 | 15 | CACACCATAG | 53.33 | −22.700705 | −22 |
| 22gfe_ | 22gf_e_ | | | Fluor 594 | | | TCGAG (SEQ | | | |
| Alex | Alexa | | | | | | ID NO: 371) | | | |
| a594 | 594 | | | | | | | | | |
| S3_2 | ser3_ | S3 | −22 | Atto647N | AXL | 12 | GCGTCCATGC | 75 | −21.68318 | −22 |
| 2gfe_ | 22gfe_ | | | | | | CG (SEQ ID NO: | | | |
| Atto647N | Atto647N | | | | | | 372) | | | |
| T3_ | ser3_ | T3 | −22 | Atto647N | AXL | 14 | CGGTACAATC | 57.14 | −22.45903 | −22 |
| 22gfe_ | 22gfe_ | | | | | | GGAC (SEQ ID | | | |
| Atto647N | Atto647N | | | | | | NO: 373) | | | |
| S15_ | ser15_ | S15 | −24 | Atto647N | EGFR | 15 | CTGGAGATCG | 60 | −24.146625 | −24 |
| 24gfe_ | 24gfe_ | | | | | | CCTTC (SEQ ID | | | |
| Atto647N | Att0647N | | | | | | NO: 374) | | | |
| T15_ | ser15_ | T15 | −24 | Atto647N | EGFR | 16 | AAAGCGTGA | 43.75 | −23.899185 | −24 |
| 24gfe_ | 24gfe_ | | | | | | TAGTGTC (SEQ | | | |
| Atto647N | Att0647N | | | | | | ID NO: 375) | | | |
| S12_ | ser12_ | S12 | −22 | Atto647N | MITF | 14 | AGCGTATGA | 50 | −21.706685 | −22 |
| 22gfe_ | 22gfe_ | | | | | | GCAGT (SEQ | | | |
| Atto647N | Att0647N | | | | | | ID NO: 376) | | | |
| T12_ | ble | T12 | −22 | Atto647N | MITF | 13 | CAACGCATCG | 61.54 | −21.682965 | −22 |
| 22gfe_ | | | | | | | GAC (SEQ ID | | | |
| Atto647N | | | | | | | NO: 377) | | | |
| S1_ | n/a | S1 | n/a | Atto647N | GFP, | 20 | TCACGACGA | 55 | | |
| read_20 | | | | | EGFR | | ATTCCCGACG | | | |
| nt | | | | | | | T (SEQ ID NO: | | | |
| | | | | | | | 378) | | | |
| S2_ | n/a | S2 | n/a | Atto647N | GFP, | 20 | CGATTCGCCA | 50 | | |
| read_20 | | | | | EGFR | | TATACTCGAC | | | |
| nt | | | | | | | (SEQ ID NO: | | | |
| | | | | | | | 379) | | | |
| S3_ | n/a | S3 | n/a | Atto647N | GFP, | 20 | ATAGCGTCCA | 55 | | |
| read_20 | | | | | EGF | | TGCCGACCTA | | | |
| nt | | | | | R | | (SEQ ID NO: | | | |
| | | | | | | | 380) | | | |
| S4_ | n/a | S4 | n/a | Atto647N | GFP, | 20 | CAACCGCAA | 50 | | |
| read_20 | | | | | EGF | | ATAGACGAG | | | |
| nt | | | | | R | | TG (SEQ ID NO: | | | |
| | | | | | | | 381) | | | |
| S5_ | n/a | S5 | n/a | Atto647N | GFP, | 20 | GACGGTAAT | 40 | | |
| read_20 | | | | | EGFR | | ATCAGTACGT | | | |
| nt | | | | | | | T (SEQ ID NO: | | | |
| | | | | | | | 382) | | | |
| S6_ | n/a | S6 | n/a | Atto647N | GFP, | 20 | ACCTACCGTA | 50 | | |
| read_20 | | | | | EGFR | | TCGCTAAGCT | | | |
| nt | | | | | | | (SEQ ID NO: | | | |
| | | | | | | | 383) | | | |
| S7_ | n/a | S7 | n/a | Atto647N | GFP, | 20 | TAGGCTCGTA | 50 | | |
| read_20 | | | | | EGFR | | TCGCTCTAGT | | | |
| nt | | | | | | | (SEQ ID NO: | | | |
| | | | | | | | 384) | | | |
| S8_ | n/a | S8 | n/a | Atto647N | GFP, | 20 | TTTCGCTGGA | 45 | | |
| read_20 | | | | | EGFR | | TAACGGTTGA | | | |
| nt | | | | | | | (SEQ ID NO: | | | |
| | | | | | | | 385) | | | |
| S9_ | n/a | S9 | n/a | Atto647N | GFP, | 20 | GAAGTCGGT | 50 | | |
| read_20 | | | | | EGFR | | ATCACGGATA | | | |
| nt | | | | | | | G (SEQ ID NO: | | | |
| | | | | | | | 386) | | | |
| S10_ | n/a | S10 | n/a | Atto647N | GFP, | 20 | CTGGAACCA | 50 | | |
| read_20 | | | | | EGFR | | ATGAGTCGCT | | | |
| nt | | | | | | | T (SEQ ID NO: | | | |
| | | | | | | | 387) | | | |
| S11_ | n/a | S11 | n/a | Atto647N | GFP, | 20 | TTGGTCGTGA | 50 | | |
| read_20 | | | | | EGFR | | TGAAGCTCCT | | | |
| nt | | | | | | | (SEQ ID NO: | | | |
| | | | | | | | 388) | | | |
| S12_ | n/a | S12 | n/a | Atto647N | GFP, | 20 | TTCGAGCGTA | 50 | | |
| read_20 | | | | | EGFR | | TGAGCAGTG | | | |
| nt | | | | | | | A (SEQ ID NO: | | | |
| | | | | | | | 389) | | | |
| S13_ | n/a | S13 | n/a | Atto647N | GFP, | 20 | GCCGATCATA | 45 | | |
| read_20 | | | | | EGFR | | TATCTCCGTT ( | | | |
| nt | | | | | | | SEQ ID NO: | | | |
| | | | | | | | 390) | | | |
| S14_ | n/a | S14 | n/a | Atto647N | GFP, | 20 | CGGACAAGA | 55 | | |
| read_20 | | | | | EGFR | | ATCCTCAGAC | | | |
| nt | | | | | | | G (SEQ ID NO: | | | |
| | | | | | | | 391) | | | |
| S15_ | n/a | S15 | n/a | Atto647N | GFP, | 20 | TCACTGGAGA | 50 | | |
| read_20 | | | | | EGFR | | TCGCCTTCTA ( | | | |
| nt | | | | | | | SEQ ID NO: | | | |
| | | | | | | | 392) | | | |

SEQ ID NOS 358-377 are sequences for strippable probes and are about 13 to about 17 nucleotides in length. SEQ ID NOS: 378-392 are all about 20 nucleotides in length. In the column labeled as Amplifier probe targeted, the letters “S” and “T” represent secondary and tertiary, respectively while the numbers next to “S” or “T” represent the numbers corresponding to amplifier set probed by the readout probe. For example, S9 stands for secondary amplifier belonging to amplifier set number 9. For readout probe sequence with 3′ amine modification SEQ ID NO: 358-392 were modified with Amino modifier (/3AmMO/).

### Conventional Single-Molecule RNA FISH Probes

The conventional 20mer single-molecule RNA FISH probes for GFP, AXL, EGFR, and DDX58 were designed as previously described (Raj et al. 2008), but selected a subset of probes not overlapping with the clampFISH 2.0 primary probes for these genes. The probes were coupled to NHS-ester dyes Cy3 (for the AXL, EGFR and DDX58 probe sets) and Alexa Fluor 555 (Invitrogen, A-20009; for the GFP probe set).

Scripts used to generate probe sequences are available at (dropbox link and/or Github link).

TABLE 4A
Sequences showing secondary and tertiary landing pad sequences.

| amplifier | | | |
|---|---|---|---|
| design & | | | |
| ordering | amplifier | secondary landing pad | tertiary landing pad |
| batch | set | (30 mer) | (30 mer) |
| 1 | 1 | AACTAACGTCGGGAATTCGTCG | TCACGCTTATTCGCATGTTTAGT |
| | | TGACTAGC (SEQ ID NO: 393) | ACGCCCT (SEQ ID NO: 408) |
| 1 | 2 | AGACGGTCGAGTATATGGCGAA | ACTTATACTCGCCGATAAAAAC |
| | | TCGTTAAG (SEQ ID NO: 394) | GTCGCACG (SEQ ID NO: 409) |
| 2 | 3 | ACAACTAGGTCGGCATGGACGC | GTGTTTTGCGTCCGATTGTACCG |
| | | TATATGAG (SEQ ID NO: 395) | ATACCAC (SEQ ID NO: 410) |
| 2 | 4 | TCGTCCACTCGTCTATTTGCGGT | ATACCTACACCGACATAGACGT |
| | | TGCGTGT (SEQ ID NO: 396) | AAGCCAGC (SEQ ID NO: 411) |
| 2 | 5 | AACCGAACGTACTGATATTACC | AGTCGGTGTCTTAGATCGGTTC |
| | | GTCGAGCA (SEQ ID NO: 397) | GCTCTTTA (SEQ ID NO: 412) |
| 2 | 6 | CTAGTAGCTTAGCGATACGGTA | GCCATTGGTATGCTATTAACCG |
| | | GGTCTAGT (SEQ ID NO: 398) | CTCGACTA (SEQ ID NO: 413) |
| 2 | 7 | CCTTAACTAGAGCGATACGAGC | TAGAACAACGGTCGATCCACTT |
| | | CTACCGAA (SEQ ID NO: 399) | CGCTCACA (SEQ ID NO: 414) |
| 3 | 8 | TGACCTCAACCGTTATCCAGCG | CTCTGTACGGTTCTATCAGATTG |
| | | AAACAAGA (SEQ ID NO: 400) | TCGAGTC (SEQ ID NO: 415) |
| 3 | 9 | TGCTTCTATCCGTGATACCGACT | TACGGTGGTGAGAAATTGGTGT |
| | | TCGTTAG (SEQ ID NO: 401) | CTGATGCG (SEQ ID NO: 416) |
| 3 | 10 | CTAACAAGCGACTCATTGGTTC | GTGTGTATTCCGCAATCCGCTG |
| | | CAGATGCA (SEQ ID NO: 402) | ATACTTGT (SEQ ID NO: 417) |
| 3 | 11 | CAACTAGGAGCTTCATCACGAC | TGTGGTTCTGCAAGATGTAGGT |
| | | CAAAGACT (SEQ ID NO: 403) | AGTGGTAC (SEQ ID NO: 418) |
| 3 | 12 | TAGGTTCACTGCTCATACGCTCG | GGTGTAAGAGTCCGATGCGTTG |
| | | AATCTTC (SEQ ID NO: 404) | TTTGTCCT (SEQ ID NO: 419) |
| 3 | 13 | TCGCAAACGGAGATATATGATC | GACAATAACCGACGATAACTCC |
| | | GGCAACAC (SEQ ID NO: 405) | GATACGCT (SEQ ID NO: 420) |
| 3 | 14 | TGTAACGTCTGAGGATTCTTGTC | TGAATGGCTCGACTATGGTGTG |
| | | CGCACAA (SEQ ID NO: 406) | TTCGTTTG (SEQ ID NO: 421) |
| 3 | 15 | CATAGTAGAAGGCGATCTCCAG | GTTCGGTTGACACTATCACGCTT |
| | | TGACTCAT (SEQ ID NO: 407) | TGAATCC (SEQ ID NO: 422) |

Table 4B show amplifier (secondary and tertiary) probe sequences and their associated primary probe sequences once fully synthesized. Amplifier probes are synthesized in the form: 5′ [5′ Hexynyl-modified 89mer]+[azido-dATP] 3′; the full 90mer sequence of amplifier probes is in the form: 5′ Hexynyl-[15mer arm][30mer landing pad][30mer landing pad]][15mer arm]-Azide 3′; primary probes are synthesized in the form: 5′ [5′ Hexynyl-modified 18mer universal sequence (first universal oligonucleotide)][30mer Amplifier-specific sequence][10mer universal adapter sequence (second universal oligonucleotide)]+[10mer universal adapter sequence (first flanking oligonucleotide)][30mer RNA-binding sequence][10mer universal sequence (second flanking oligonucleotide)]+[azido-dATP] 3′; −x denotes bases hybridizing to a target RNA.

TABLE 4B

| amplifier | | | | |
|---|---|---|---|---|
| design & | | secondary full 90 mer | tertiary full 90 mer | primary probe full- |
| ordering | amplifier | sequence (with 3′ azido- | sequence (with 3′ azido- | length sequence |
| batch | set | dATP added) | dATP added) | (109 mer) |
| 1 | 1 | tgcgaataagcgtgaAAC | ttcccgacgttagttTCAC | agacattctcgtcaagatT |
| | | TAACGTCGGGAA | GCTTATTCGCAT | CACGCTTATTCG |
| | | TTCGTCGTGACT | GTTTAGTACGCC | CATGTTTAGTAC |
| | | AGCAACTAACGT | CTTCACGCTTATT | GCCCTctgagtgttgA |
| | | CGGGAATTCGTC | CGCATGTTTAGT | AGTGACTGTnnnn |
| | | GTGACTAGCagggc | ACGCCCTgctagtcac | nnnnnnnnnnnnnnnnn |
| | | gtactaaaca (SEQ ID | gacgaa (SEQ ID | nnnnnnnnnACATC |
| | | NO: 423) | NO: 438) | ATAGTa (SEQ ID |
| | | | | NO: 453) |
| 1 | 2 | tcggcgagtataagtAGA | tatactcgaccgtctACTT | agacattctcgtcaagatA |
| | | CGGTCGAGTATA | ATACTCGCCGAT | CTTATACTCGCC |
| | | TGGCGAATCGTT | AAAAACGTCGCA | GATAAAAACGTC |
| | | AAGAGACGGTCG | CGACTTATACTC | GCACGctgagtgttgA |
| | | AGTATATGGCGA | GCCGATAAAAAC | AGTGACTGTnnnn |
| | | ATCGTTAAGcgtgc | GTCGCACGcttaacg | nnnnnnnnnnnnnnnnn |
| | | gacgttttta (SEQ ID | attcgcca (SEQ ID | nnnnnnnnnACATC |
| | | NO: 424) | NO: 439) | ATAGTa (SEQ ID |
| | | | | NO: 454) |
| 2 | 3 | toggacgcaaaacacAC | tgccgacctagttgtGTG | agacattctcgtcaagatG |
| | | AACTAGGTCGGC | TTTTGCGTCCGAT | TGTTTTGCGTCCG |
| | | ATGGACGCTATA | TGTACCGATACC | ATTGTACCGATA |
| | | TGAGACAACTAG | ACGTGTTTTGCG | CCACctgagtgttgAA |
| | | GTCGGCATGGAC | TCCGATTGTACC | GTGACTGTnnnnnn |
| | | GCTATATGAGgtg | GATACCACctcatata | nnnnnnnnnnnnnnnnn |
| | | gtatcggtacaa (SEQ ID | gcgtcca (SEQ ID | nnnnnnnACATCAT |
| | | NO: 425) | NO: 440) | AGTa (SEQ ID |
| | | | | NO: 455) |
| 2 | 4 | tgtcggtgtaggtatTCG | tagacgagtggacgaAT | agacattctcgtcaagatA |
| | | TCCACTCGTCTAT | ACCTACACCGAC | TACCTACACCGA |
| | | TTGCGGTTGCGT | ATAGACGTAAGC | CATAGACGTAAG |
| | | GTTCGTCCACTC | CAGCATACCTAC | CCAGCctgagtgttgA |
| | | GTCTATTTGCGG | ACCGACATAGAC | AGTGACTGTnnnn |
| | | TTGCGTGTgctggctt | GTAAGCCAGCaca | nnnnnnnnnnnnnnnnn |
| | | acgtcta (SEQ ID | cgcaaccgcaaa (SEQ | nnnnnnnnnACATC |
| | | NO: 426) | ID NO: 441) | ATAGTa (SEQ ID |
| | | | | NO: 456) |
| 2 | 5 | tctaagacaccgactAAC | tcagtacgttcggttAGT | agacattctcgtcaagatA |
| | | CGAACGTACTGA | CGGTGTCTTAGA | GTCGGTGTCTTA |
| | | TATTACCGTCGA | TCGGTTCGCTCTT | GATCGGTTCGCT |
| | | GCAAACCGAACG | TAAGTCGGTGTC | CTTTActgagtgttgA |
| | | TACTGATATTAC | TTAGATCGGTTC | AGTGACTGTnnnn |
| | | CGTCGAGCAtaaag | GCTCTTTAtgctcgac | nnnnnnnnnnnnnnnnn |
| | | agcgaaccga (SEQ ID | ggtaata (SEQ ID | nnnnnnnnnACATC |
| | | NO: 427) | NO: 442) | ATAGTa (SEQ ID |
| | | | | NO: 457) |
| 2 | 6 | tagcataccaatggcCTA | tcgctaagctactagGCC | agacattctcgtcaagatG |
| | | GTAGCTTAGCGA | ATTGGTATGCTA | CCATTGGTATGC |
| | | TACGGTAGGTCT | TTAACCGCTCGA | TATTAACCGCTC |
| | | AGTCTAGTAGCT | CTAGCCATTGGT | GACTActgagtgttgA |
| | | TAGCGATACGGT | ATGCTATTAACC | AGTGACTGTnnnn |
| | | AGGTCTAGTtagtcg | GCTCGACTAactag | nnnnnnnnnnnnnnnnn |
| | | agcggttaa (SEQ ID | acctaccgta (SEQ ID | nnnnnnnnnACATC |
| | | NO: 428) | NO: 443) | ATAGTa (SEQ ID |
| | | | | NO: 458) |
| 2 | 7 | tcgaccgttgttctaCCTT | tcgctctagttaaggTAG | agacattctcgtcaagatT |
| | | AACTAGAGCGAT | AACAACGGTCGA | AGAACAACGGTC |
| | | ACGAGCCTACCG | TCCACTTCGCTC | GATCCACTTCGC |
| | | AACCTTAACTAG | ACATAGAACAAC | TCACActgagtgttgA |
| | | AGCGATACGAGC | GGTCGATCCACT | AGTGACTGTnnnn |
| | | CTACCGAAtgtgagc | TCGCTCACAttcggt | nnnnnnnnnnnnnnnnn |
| | | gaagtgga (SEQ ID | aggctcgta (SEQ ID | nnnnnnnnnACATC |
| | | NO: 429) | NO: 444) | ATAGTa (SEQ ID |
| | | | | NO: 459) |
| 3 | 8 | tagaaccgtacagagTG | taacggttgaggtcaCTC | agacattctcgtcaagatC |
| | | ACCTCAACCGTT | TGTACGGTTCTA | TCTGTACGGTTCT |
| | | ATCCAGCGAAAC | TCAGATTGTCGA | ATCAGATTGTCG |
| | | AAGATGACCTCA | GTCCTCTGTACG | AGTCctgagtgttgAA |
| | | ACCGTTATCCAG | GTTCTATCAGAT | GTGACTGTnnnnnn |
| | | CGAAACAAGAgac | TGTCGAGTCtcttgtt | nnnnnnnnnnnnnnnnn |
| | | tcgacaatctga (SEQ ID | tcgctgga (SEQ ID | nnnnnnnACATCAT |
| | | NO: 430) | NO: 445) | AGTa (SEQ ID |
| | | | | NO: 460) |
| 3 | 9 | tttctcaccaccgtaTGCT | tcacggatagaagcaTAC | agacattctcgtcaagatT |
| | | TCTATCCGTGAT | GGTGGTGAGAAA | ACGGTGGTGAGA |
| | | ACCGACTTCGTT | TTGGTGTCTGAT | AATTGGTGTCTG |
| | | AGTGCTTCTATC | GCGTACGGTGGT | ATGCGctgagtgttgA |
| | | CGTGATACCGAC | GAGAAATTGGTG | AGTGACTGTnnnn |
| | | TTCGTTAGcgcatca | TCTGATGCGctaac | nnnnnnnnnnnnnnnnn |
| | | gacaccaa (SEQ ID | gaagtcggta (SEQ ID | nnnnnnnnnACATC |
| | | NO: 431) | NO: 446) | ATAGTa (SEQ ID |
| | | | | NO: 461) |
| 3 | 10 | ttgcggaatacacacCTA | tgagtcgcttgttagGTG | agacattctcgtcaagatG |
| | | ACAAGCGACTCA | TGTATTCCGCAA | TGTGTATTCCGC |
| | | TTGGTTCCAGAT | TCCGCTGATACT | AATCCGCTGATA |
| | | GCACTAACAAGC | TGTGTGTGTATTC | CTTGTctgagtgttgA |
| | | GACTCATTGGTT | CGCAATCCGCTG | AGTGACTGTnnnn |
| | | CCAGATGCAacaag | ATACTTGTtgcatctg | nnnnnnnnnnnnnnnnn |
| | | tatcagcgga (SEQ ID | gaaccaa (SEQ ID | nnnnnnnnnACATC |
| | | NO: 432) | NO: 447) | ATAGTa (SEQ ID |
| | | | | NO: 462) |
| 3 | 11 | tcttgcagaaccacaCAA | tgaagctcctagttgTGT | agacattctcgtcaagatT |
| | | CTAGGAGCTTCA | GGTTCTGCAAGA | GTGGTTCTGCAA |
| | | TCACGACCAAAG | TGTAGGTAGTGG | GATGTAGGTAGT |
| | | ACTCAACTAGGA | TACTGTGGTTCT | GGTACctgagtgttgA |
| | | GCTTCATCACGA | GCAAGATGTAGG | AGTGACTGTnnnn |
| | | CCAAAGACTgtacc | TAGTGGTACagtctt | nnnnnnnnnnnnnnnnn |
| | | actacctaca (SEQ ID | tggtcgtga (SEQ ID | nnnnnnnnnACATC |
| | | NO: 433) | NO: 448) | ATAGTa (SEQ ID |
| | | | | NO: 463) |
| 3 | 12 | toggactcttacaccTAG | tgagcagtgaacctaGGT | agacattctcgtcaagatG |
| | | GTTCACTGCTCA | GTAAGAGTCCGA | GTGTAAGAGTCC |
| | | TACGCTCGAATC | TGCGTTGTTTGTC | GATGCGTTGTTT |
| | | TTCTAGGTTCACT | CTGGTGTAAGAG | GTCCTctgagtgttgA |
| | | GCTCATACGCTC | TCCGATGCGTTG | AGTGACTGTnnnn |
| | | GAATCTTCaggacaa | TTTGTCCTgaagattc | nnnnnnnnnnnnnnnnn |
| | | acaacgca (SEQ ID | gagcgta (SEQ ID | nnnnnnnnnACATC |
| | | NO: 434) | NO: 449) | ATAGTa (SEQ ID |
| | | | | NO: 464) |
| 3 | 13 | tcgtcggttattgtcTCGC | tatctccgtttgcgaGAC | agacattctcgtcaagatG |
| | | AAACGGAGATAT | AATAACCGACGA | ACAATAACCGAC |
| | | ATGATCGGCAAC | TAACTCCGATAC | GATAACTCCGAT |
| | | ACTCGCAAACGG | GCTGACAATAAC | ACGCTctgagtgttgA |
| | | AGATATATGATC | CGACGATAACTC | AGTGACTGTnnnn |
| | | GGCAACACagcgtat | CGATACGCTgtgttg | nnnnnnnnnnnnnnnnn |
| | | cggagtta (SEQ ID | ccgatcata (SEQ ID | nnnnnnnnnACATC |
| | | NO: 435) | NO: 450) | ATAGTa (SEQ ID |
| | | | | NO: 465) |
| 3 | 14 | tagtcgagccattcaTGT | tcctcagacgttacaTGA | agacattctcgtcaagatT |
| | | AACGTCTGAGGA | ATGGCTCGACTA | GAATGGCTCGAC |
| | | TTCTTGTCCGCAC | TGGTGTGTTCGTT | TATGGTGTGTTC |
| | | AATGTAACGTCT | TGTGAATGGCTC | GTTTGctgagtgttgA |
| | | GAGGATTCTTGT | GACTATGGTGTG | AGTGACTGTnnnn |
| | | CCGCACAAcaaacg | TTCGTTTGttgtgcgg | nnnnnnnnnnnnnnnnn |
| | | aacacacca (SEQ ID | acaagaa (SEQ ID | nnnnnnnnnACATC |
| | | NO: 436) | NO: 451) | ATAGTa (SEQ ID |
| | | | | NO: 466) |
| 3 | 15 | tagtgtcaaccgaacCAT | tcgccttctactatgGTTC | agacattctcgtcaagatG |
| | | AGTAGAAGGCGA | GGTTGACACTAT | TTCGGTTGACAC |
| | | TCTCCAGTGACT | CACGCTTTGAAT | TATCACGCTTTG |
| | | CATCATAGTAGA | CCGTTCGGTTGA | AATCCctgagtgttgA |
| | | AGGCGATCTCCA | CACTATCACGCT | AGTGACTGTnnnn |
| | | GTGACTCATggattc | TTGAATCCatgagtc | nnnnnnnnnnnnnnnnn |
| | | aaagcgtga (SEQ ID | actggaga (SEQ ID | nnnnnnnnnACATC |
| | | NO: 437) | NO: 452) | ATAGTa (SEQ ID |
| | | | | NO: 467) |

Amplifier (secondary and tertiary) probe sequences and their associated primary probe sequences once fully synthesized:
amplifier probes are synthesized in the form: 5′ [5′ Hexynyl-modified 89 mer] + [azido-dATP] 3′; the full 90 mer sequence of amplifier probes is in the form: 5′ Hexynyl-[15 mer arm][30 mer landing pad] [[30 mer landing pad]][15 mer arm]-Azide 3′; primary probes are synthesized in the form: 5′ [5′ Hexynyl-modified 18 mer universal sequence][30 mer Amplifier-specific sequence][10 mer universal adapter sequence] + [10 mer universal adapter sequence][30 mer RNA-binding sequence][10 mer universal sequence] + [azido-dATP] 3′; n denotes bases hybridizing to a target RNA

Table 5: All conventional single-molecule RNA FISH probes were ordered from Biosearch, with 3′ Amine modifications and delivered at 100 μM concentration in water. Each probe was then coupled to a NHS-Ester dye and purified using HPLC

| | Dye | | |
|---|---|---|---|
| Gene | coupled to | Sequence | Sequence Name |
| d2EGFP | Alexa | CACCCCGGTGAACAGCT (SEQ ID | gfp_47 |
| | Fluor | NO: 468) | |
| | 555 | | |
| d2EGFP | Alexa | GGCCAGGGCACGGGCAG (SEQ ID | gfp_40 |
| | Fluor | NO: 469) | |
| | 555 | | |
| d2EGFP | Alexa | TCAGGGTGGTCACGAGG (SEQ ID | gfp_39 |
| | Fluor | NO: 470) | |
| | 555 | | |
| d2EGFP | Alexa | TGAAGAAGTCGTGCTGC (SEQ ID | gfp_36 |
| | Fluor | NO: 471) | |
| | 555 | | |
| d2EGFP | Alexa | TGCCGTCGTCCTTGAAG (SEQ ID | gfp_33 |
| | Fluor | NO: 472) | |
| | 555 | | |
| d2EGFP | Alexa | GCTCGATGCGGTTCACC (SEQ ID | gfp_30 |
| | Fluor | NO: 473) | |
| | 555 | | |
| d2EGFP | Alexa | CTTGAAGTCGATGCCCT (SEQ ID | gfp_29 |
| | Fluor | NO: 474) | |
| | 555 | | |
| d2EGFP | Alexa | ATAGACGTTGTGGCTGT (SEQ ID | gfp_26 |
| | Fluor | NO: 475) | |
| | 555 | | |
| d2EGFP | Alexa | TTCTGCTTGTCGGCCAT (SEQ ID | gfp_25 |
| | Fluor | NO: 476) | |
| | 555 | | |
| d2EGFP | Alexa | AGTTCACCTTGATGCCG (SEQ ID | gfp_24 |
| | Fluor | NO: 477) | |
| | 555 | | |
| d2EGFP | Alexa | TCTGCTGGTAGTGGTCG (SEQ ID | gfp_21 |
| | Fluor | NO: 478) | |
| | 555 | | |
| d2EGFP | Alexa | ACTGGGTGCTCAGGTAG (SEQ ID | gfp_18 |
| | Fluor | NO: 479) | |
| | 555 | | |
| d2EGFP | Alexa | GGGGTCTTTGCTCAGGG (SEQ ID | gfp_17 |
| | Fluor | NO: 480) | |
| | 555 | | |
| d2EGFP | Alexa | GAGAGTGATCCCGGCGG (SEQ ID | gfp_14 |
| | Fluor | NO: 481) | |
| | 555 | | |
| d2EGFP | Alexa | TTGTACAGCTCGTCCAT (SEQ ID | gfp_13 |
| | Fluor | NO: 482) | |
| | 555 | | |
| AXL | Cy3 | CTCTTGCCAATGCACGAAGG (SEQ | AXL_CDS_3 |
| | | ID NO: 483) | |
| AXL | Cy3 | TATGTCCATTAGCACCTCTG (SEQ | AXL_CDS_4 |
| | | ID NO: 484) | |
| AXL | Cy3 | TGGGATGGTCAAATTCCTTC (SEQ | AXL_CDS_13 |
| | | ID NO: 485) | |
| AXL | Cy3 | TCAGATACTCCATGCCACTG (SEQ | AXL_CDS_21 |
| | | ID NO: 486) | |
| AXL | Cy3 | CGGTGTATGAATCTCTTGGT (SEQ | AXL_CDS_22 |
| | | ID NO: 487) | |
| AXL | Cy3 | CCATTGTAGATCTTCTTGGA (SEQ | AXL_CDS_23 |
| | | ID NO: 488) | |
| AXL | Cy3 | TGGCATCTTGGCGATACGTC (SEQ | AXL_CDS_24 |
| | | ID NO: 489) | |
| AXL | Cy3 | AAGGACCACACATCGCTCTT (SEQ | AXL_CDS_27 |
| | | ID NO: 490) | |
| AXL | Cy3 | ATAAATCTCGCTGTTCTCCA (SEQ | AXL_CDS_28 |
| | | ID NO: 491) | |
| AXL | Cy3 | CCGCAGCTCTGTAAAACTTG (SEQ | AXL_CDS_30 |
| | | ID NO: 492) | |
| AXL | Cy3 | TCAGTGTGTTCTCCAAATCT (SEQ | AXL_CDS_31 |
| | | ID NO: 493) | |
| AXL | Cy3 | ACATAGCGTCCAGCAGGATG (SEQ | AXL_CDS_33 |
| | | ID NO: 494) | |
| AXL | Cy3 | TAGGGGTTGTGGAAGGGCAG (SEQ | AXL_CDS_34 |
| | | ID NO: 495) | |
| AXL | Cy3 | GCACAGAGAAGGGGAGGGAC (SEQ | AXL_3UTR_5 |
| | | ID NO: 496) | |
| AXL | Cy3 | CTGCTTTCAAGGTGATGCTA (SEQ | AXL_3UTR_6 |
| | | ID NO: 497) | |
| AXL | Cy3 | TACAGCCAGAGAATCTAGAC (SEQ | AXL_3UTR_19 |
| | | ID NO: 498) | |
| AXL | Cy3 | CTTGGAGCATCTGATCTAGA (SEQ | AXL_3UTR_25 |
| | | ID NO: 499) | |
| AXL | Cy3 | ACAACTGAGTGTGAGAGTCT (SEQ | AXL_3UTR_29 |
| | | ID NO:500) | |
| AXL | Cy3 | CAAAGAACCTAAAGGTGTCC (SEQ | AXL_3UTR_30 |
| | | ID NO: 501) | |
| AXL | Cy3 | CACAGTTGGGGAGGAGGAAG (SEQ | AXL_3UTR_38 |
| | | ID NO: 502) | |
| AXL | Cy3 | AAAGGTGGGAGATCGTCCAT (SEQ | AXL_3UTR_41 |
| | | ID NO: 503) | |
| AXL | Cy3 | AAGGTGAGGGATTGGCTCAC (SEQ | AXL_3UTR_42 |
| | | ID NO: 504) | |
| AXL | Cy3 | AGAGTCCACACTCTGTACTC (SEQ | AXL_3UTR_43 |
| | | ID NO: 505) | |
| EGFR | Cy3 | GTAAATTTCTCATGGGCAGC (SEQ | EGFR_CDS_10 |
| | | ID NO: 506) | |
| EGFR | Cy3 | ACAGCTTGGATCACACTTTT (SEQ | EGFR_CDS_11 |
| | | ID NO: 507) | |
| EGFR | Cy3 | GTCAGTTTCTGGCAGTTCTC (SEQ | EGFR_CDS_12 |
| | | ID NO: 508) | |
| EGFR | Cy3 | CTGCTGGGCACAGATGATTT (SEQ | EGFR_CDS_13 |
| | | ID NO: 509) | |
| EGFR | Cy3 | TGGCACCAAAGCTGTATTTG (SEQ | EGFR_CDS_17 |
| | | ID NO: 510) | |
| EGFR | Cy3 | CACTTCTTACACTTGCGGAC (SEQ | EGFR_CDS_19 |
| | | ID NO: 511) | |
| EGFR | Cy3 | GCGTAATCCCAAGGATGTTA (SEQ | EGFR_CDS_33 |
| | | ID NO: 512) | |
| EGFR | Cy3 | AGCTGTTTTCACCTCTGTTG (SEQ | EGFR_CDS_35 |
| | | ID NO: 513) | |
| EGFR | Cy3 | TGACATTCCGGCAAGAGACG (SEQ | EGFR_CDS_36 |
| | | ID NO: 514) | |
| EGFR | Cy3 | CTGTGCAGGTGATGTTCATG (SEQ | EGFR_CDS_40 |
| | | ID NO: 515) | |
| EGFR | Cy3 | CACACTGGATACAGTTGTCT (SEQ | EGFR_CDS_41 |
| | | ID NO: 516) | |
| EGFR | Cy3 | TGTTGTTTTCTCCCATGACT (SEQ | EGFR_CDS_42 |
| | | ID NO: 517) | |
| EGFR | Cy3 | ATTCGTTGGACAGCCTTCAA (SEQ | EGFR_CDS_44 |
| | | ID NO: 518) | |
| EGFR | Cy3 | CGATGGACGGGATCTTAGGC (SEQ | EGFR_CDS_45 |
| | | ID NO: 519) | |
| EGFR | Cy3 | CTTATACACCGTGCCGAACG (SEQ | EGFR_CDS_50 |
| | | ID NO: 520) | |
| EGFR | Cy3 | ATCTGCACACACCAGTTGAG (SEQ | EGFR_CDS_58 |
| | | ID NO: 521) | |
| EGFR | Cy3 | GTGTTTTCACCAGTACGTTC (SEQ | EGFR_CDS_59 |
| | | ID NO: 522) | |
| EGFR | Cy3 | TCATATGGCTTGGATCCAAA (SEQ | EGFR_CDS_65 |
| | | ID NO: 523) | |
| EGFR | Cy3 | TCGTCCATGTCTTCTTCATC (SEQ | EGFR_CDS_73 |
| | | ID NO: 524) | |
| EGFR | Cy3 | AATTGTTGCTGGTTGCACTC (SEQ | EGFR_CDS_74 |
| | | ID NO: 525) | |
| EGFR | Cy3 | GGAGGAAGGTGTCGTCTATG (SEQ | EGFR_CDS_78 |
| | | ID NO: 526) | |
| DDX58 | Cy3 | gtgcctcactagctttaaag (SEQ ID NO: 527) | DDX58_1 |
| DDX58 | Cy3 | tataatcctggaaggcttgc (SEQ ID NO: 528) | DDX58_2 |
| DDX58 | Cy3 | agaaaaagtgtggcagcctc (SEQ ID NO: 529) | DDX58_3 |
| DDX58 | Cy3 | atccaaaaagccacggaacc (SEQ ID NO: 530) | DDX58_4 |
| DDX58 | Cy3 | aagacaactttccccttttg (SEQ ID NO: 531) | DDX58_9 |
| DDX58 | Cy3 | gctatcactgacgcatcaag (SEQ ID NO: 532) | DDX58_18 |
| DDX58 | Cy3 | ctccagattgtgtttgactg (SEQ ID NO: 533) | DDX58_19 |
| DDX58 | Cy3 | atgcatgctttctgaactgt (SEQ ID NO: 534) | DDX58_22 |
| DDX58 | Cy3 | gggtttaagtggtactcttc (SEQ ID NO: 535) | DDX58_27 |
| DDX58 | Cy3 | tggctttgaatgcatccaat (SEQ ID NO: 536) | DDX58_29 |
| DDX58 | Cy3 | cttcatcagcaactgaggtg (SEQ ID NO: 537) | DDX58_30 |
| DDX58 | Cy3 | tacaccagcattactagtca (SEQ ID NO: 538) | DDX58_32 |
| DDX58 | Cy3 | tgtaacatgccaaggctttg (SEQ ID NO: 539) | DDX58_35 |
| DDX58 | Cy3 | gtctactcacaaagcattcc (SEQ ID NO: 540) | DDX58_37 |
| DDX58 | Cy3 | aaaactgctttggcttggga (SEQ ID NO: 541) | DDX58_38 |
| DDX58 | Cy3 | cctgtagctgaagattgagg (SEQ ID NO: 542) | DDX58_41 |
| DDX58 | Cy3 | tactcagtgctgtgattcac (SEQ ID NO: 543) | DDX58_42 |
| DDX58 | Cy3 | attactgtgtgtttgttggc (SEQ ID NO: 544) | DDX58_43 |
| DDX58 | Cy3 | ttgctacaccaggaacatga (SEQ ID NO: 545) | DDX58_44 |
| DDX58 | Cy3 | gctttatgttctcatgttct (SEQ ID NO: 546) | DDX58_45 |
| DDX58 | Cy3 | gaccactgtagagtggatac (SEQ ID NO: 547) | DDX58_46 |
| DDX58 | Cy3 | aaccttgctaagtgacacgt (SEQ ID NO: 548) | DDX58_47 |
| DDX58 | Cy3 | ctcagatacttgtttccacg (SEQ ID NO: 549) | DDX58_48 |

### Cell Culture and Tissue Processing

The WM989 A6-G3 human melanoma cell line, first described in (Shaffer et al. 2017) was derived from WM989 cells that were twice isolated from a single cell and expanded. WM989 A6-G3 H2B-GFP cells were derived by transducing WM989 A6-G3 cells with 60 μL Lenti_EFS (benchling.com/s/seq-6Jv3Rmebv1nIevxPfYQ6/edit), isolating a single cell, and expanding this clone (Clone A11). Both lines were cultured in Tu2% media (80% MCDB 153, 10% Leibovitz's L-15, 2% FBS, 2.4 mM CaCl₂), 50 U/mL penicillin, and 50 μg/mL streptomycin). WM989 A6-G3 RC4 cells were derived by treating WM989 A6-G3 cells with 1 μM vemurafenib in Tu2%, isolating a single drug-resistant colony, and culturing these cells in 1 μM vemurafenib in Tu2% (Goyal et al. 2021). All cell lines were passaged with 0.05% trypsin-EDTA (Gibco, 25300120).

For the amplifier screen and pooled amplification experiment, WM989 A6-G3 H2B-GFP and WM989 A6-G3 RC3 cells were mixed together and plated on coverslips (VWR, 16004-098, 24×50 mm, No. 1 coverglass) with 24-well silicone isolators (Grace Bio-Labs, 665108). For the readout probe stripping experiment, conventional single-molecule RNA FISH comparison experiment, and the amplification characterization experiment, WM989 A6-G3 or WM989 A6-G3 RC4 cells were plated into separate wells of an 8-well chambers (Lab-tek, 155411, No. 1 coverglass). For the high-throughput profiling experiment, WM989 A6-G3 cells were plated into 5 wells and WM989 A6-G3 RC4 cells into 1 well of a 6-well plate (Cellvis, P06-1.5H-N, No. 1.5 coverglass)), and allowed them to grow out for 6 days (2-3 cell divisions for WM989 A6-G3 cells) before fixation.

### Details of Tissue Experiment Sample Preparation

The cell lines were fixed at room temperature by rinsing cells once in 1×PBS (Invitrogen, AM9624), incubating for 10 minutes in 3.7% formaldehyde (Sigma-Aldrich, F1635-500ML) in 1×PBS, then rinsing twice in 1×PBS. Cells were permeabilized in 70% ethanol and placed at 4° C. for at least 8 hours. Nuclease-free water (Invitrogen, 4387936) was used in all buffers used for fixation onwards, including permeabilization, probe synthesis, and all RNA FISH steps.

For the fresh frozen tissue experiment, a melanoma xenograft tumor was taken from experiments described in (Torre et al. 2021). Briefly, human WM989-A6-G3-Cas9-5a3 cells (without a genetic knockout), derived by isolating and expanding a single WM989 A6-G3 cell, were injected into 8-week-old NOD/SCID mice (Charles River Laboratories) and fed AlN-76A chow containing 417 mg kg-1 PLX4720. Once the tumor reached 1,500 mm³ the mouse was euthanized, and the tumor tissue was dissected and placed in a cryomold with optimal cutting temperature compound (TissueTek, 4583), frozen in liquid nitrogen, and then stored at −80° C. Tumors were then sectioned on a cryostat to 6 μm thickness, placed onto a microscope slide (Fisher Scientific, 6776214), fixed and permeabilized with the same protocol used for cell lines while in LockMailer slide jars (Fisher Scientific, 50-340-92), and then stored at 4° C.

For the formalin-fixed paraffin embedded (FFPE) tissue experiment, clampFISH 2.0 was performed in two patient-derived xenografts (PDXs), with sample identifiers WM4505-1 (used in replicates 1 and 2) and WM4298-2 (used in replicate 2). The PDXs were each derived from a tumor from a metastatic site of a male patient diagnosed with AJCC Stage IV melanoma. PDX WM4505-1 was derived from an unknown metastatic site in a patient previously treated with combination dabrafenib and trametinib with a mixed response, and whose primary tumor site was the scalp. PDX WM4298-2 was derived from a left back metastatic site in a patient previously treated with vemurafenib, which was discontinued due to an allergic reaction, and whose primary tumor site is unknown. Each PDX was grown out in male NSG mice that were 6-8 weeks old at the time of implantation, with passages performed via subcutaneous implantation of a fragment of the PDX into another mouse. The PDXs were grown for a total of 4 passages (for WM4505-1) or 3 passages (for WM4298-2), where after the first passage, the mice were continuously fed chow containing BRAF/MEK inhibitors (PLX4720 200 ppm+PD-0325901 7 ppm, chemical additive diet, Research Diets, New Brunswick, NJ). Finally, a piece of about 3×3×3 mm³ of each PDX tumor was implanted into an 6-8 week old male NSG mouse that, once the tumor was palpable, was fed chow containing the BRAF/MEK inhibitors. Tumor size was assessed once weekly by caliper measurements (length×width 2/2). When the tumors reached 1,000 mm³ or when necessary for animal welfare, the tumor was harvested and immediately placed in 10% neutral buffered formalin overnight (less than 48 hrs), washed once with 1×PBS, and stored in 70% ethanol at room temperature. Next, the fixed tumor samples were embedded in paraffin, sectioned to 5 μm thickness, and placed on a microscope slide. To avoid exposure to the air, the samples were sealed with a thin layer of paraffin, then stored at room temperature. For both the fresh frozen tissue and the FFPE tissue samples, the samples' slides were placed in 2×SSC for 1-5 minutes, in 8% sodium dodecyl sulfate (Sigma-Aldrich, 75746-250G; dissolved in nuclease-free water) for 2 minutes, and then into 2×SSC for up to 2 hours, after which began the primary probe steps. The clampFISH 2.0 steps were performed in parallel for both types of samples (fresh frozen and FFPE) in two separate experimental replicates (replicate 1: fresh frozen mouse #8948 and FFPE PDX WM4505-1; replicate 2: fresh frozen samples #8948 and #8947 and FFPE samples WM4505-1 and WM4298-2).

clampFISH 2.0 Protocol
clampFISH 2.0 Primary Probe Steps

ClampFISH 2.0 was performed in 8-well chambers as follows. First, the 70% ethanol (or 2×SSC for tissue sections) was aspirated, rinsed with 10% wash buffer (10% formamide, 2×SSC), then washed with 40% wash buffer (40% formamide, 2×SSC) for 5-10 minutes. The primary probes were mixed with 40% hybridization buffer (40% formamide, 10% dextran sulfate, 2×SSC) such that each probe's final concentration was 0.1 ng/μl (˜2.8 nM), this mixture was added to the well, covered and spread out with a coverslip, and then incubated overnight (10 or more hours) in a humidified container at 37° C. Only a single primary probe set was hybridized per well with the amplifier screen experiment (GFP or EGFR probe sets) and the pooled amplification experiment (GFP probe set). For all other experiments 10 primary probe sets were hybridized together.

The following day, all wash buffers (10% wash buffer, 30% wash buffer (30% formamide, 2×SSC), and 40% wash buffer) were prewarmed to 37° C. The warm 10% wash buffer was first added, coverslips were removed and the solution was aspirated, and washed again with warm 10% wash buffer washess were performed twice for 20 minutes with warm 40% wash buffer on a hotplate set to 37° C. (the temperature setting used throughout the protocol). After removing the chamber from the hotplate, 10% wash buffer was added before beginning the amplification steps.

clampFISH 2.0 Amplification Steps

For amplification, all the secondary probes were first mixed with 10% hybridization buffer with Triton-X (10% formamide, 10% dextran sulfate, 2×SSC, and 0.1% Triton-X (Sigma-Aldrich, T8787-100ML)) to a final ˜20 nM concentration per probe (range: ˜ 13 nM to 25 nM) with a circularizer oligonucleotide at a 40 nM final concentration. Also, mixed together were all tertiary probes with 10% hybridization buffer with Triton-X at the same concentrations, but without the circularizer oligonucleotide. In preparation for multiple click reaction steps, each tube was prepared with an appropriate volume of pre-warmed 2×SSC with Triton-X and DMSO (2×SSC, 0.25% Triton-X, 10% dimethyl sulfoxide) for the amplification step, and was warmed to 37° C. Sodium ascorbate (Acros, AC352680050) was also aliquoted into 1.5 mL tubes, ready to be dissolved fresh with each click step. A CuSO₄ (Fisher Scientific, S25289) and BTTAA (Jena Bioscience, CLK-067-100) mixture was prepared in a 1:2 CuSO₄:BTTAA molar ratio, enough to use for all the click reactions throughout the rounds of amplification.

The secondary probe-containing 10% hybridization buffer with Triton-X were added to the well, covered with a coverslip, and incubated for 30 minutes in a 37° C. incubator. After taking the chamber out of the incubator, warm 10% wash buffer was added, the coverslips were removed 2×1 minute washes were performed with warm 10% wash buffer, and then again another was was performed with warm 10% wash buffer for 10 minutes on the hotplate. The chamber was then taken off the hotplate and room-temperature 2×SSC was added before the click reaction. The click reaction mixture was then prepared by first mixing the CuSO₄ and BTTAA mixture with the pre-warmed 2×SSC with Triton-X and DMSO buffer. Working quickly, nuclease-free water was added to an ascorbic acid aliquot and vortexed until dissolved.

The 2×SSC solution was aspirated from the well plate, and aqueous sodium ascorbate was quickly added to the CuSO₄+BTTAA+2×SSC with Triton-X and DMSO mixture (final concentrations: 150 μM CuSO₄, 300 μM BTTAA, 5 mM sodium ascorbate, ˜2×SSC, ˜0.25% Triton-X, ˜10% DMSO) and briefly mixed by swirling the tube by hand. The click reaction solution was immediately added to the wells and incubated on the hotplate for 10 minutes. Next, the click reaction mixture was aspirated and the sample was washed with warm 30% wash buffer for 5 minutes on the hotplate. The above steps (amplifier probe hybridization, 10% wash buffer steps, click reaction, and 30% wash buffer step) constitutes a single round of amplification, and takes about 1 hour when accounting for pipetting time.

Before beginning the next round of amplification, the 30% wash buffer was replaced with warm 10% wash buffer. (If, alternatively, a breakpoint was needed in between rounds of amplification, the 30% wash buffer was instead replaced with 2×SSC and stored the sample at room temperature for up to 2 hours or at 4° C. for up to a day). The next round of amplification was performed using tertiary probes instead of secondary probes. The completion of the primary step was dubbed as having performed clampFISH 2.0 to “round 1”, the first secondary step as “round 2”, the first tertiary step as “round 3”, the next secondary step as “round 4”, and so on. All amplifications were ran to round 8, involving 1 primary probe round and 7 amplification rounds, except where noted differently. At the end of the last amplification round, the sample was placed at 4° C. in 2×SSC until the readout probe steps (typically the samples were stored overnight for readout and imaging the subsequent day).

The amplifier screen experiment and the pooled amplification experiment, were performed with conventional single-molecule RNA FISH per (Raj et al. 2008) by first rinsing briefly with 10% wash buffer, adding GFP or EGFR probes as well as a 20 nucleotide secondary-targeting readout probe at 4 nM final concentration in 10% hybridization buffer (10% formamide, 10% dextran sulfate, 2×SSC), covering with a coverslip, placing in a humidified container and incubating overnight in at 37° C., adding 10% wash buffer to remove the coverslip, washing 2×30 minutes in 10% wash buffer in a 37° C. incubator, while adding 50 ng/mL of the nuclear stain 4′,6-diamidino-2-phenylindole (DAPI) to the second wash, after which further readout probe steps were not carried out. For the wash and click steps that use a hotplate, in these two experiments a 37° C. incubator or bead bath was instead used, with the sample in a LockMailer slide jar submerged in the appropriate buffer.

For the high-throughput profiling experiment in a 6-well plate, the use of a hotplate was replaced with a 37° C. incubator; and further increased the incubation time of the 10 minute wash in 10% wash buffer, the 10 minute click reaction, and all steps in 30% wash buffer by an additional 4 minutes to accommodate the longer time to warm-up.

In an experiment assessing a one-pot amplification protocol (adding secondary probes, tertiary probes, and the click reagents simultaneously), first added was one of two buffers: a buffer with dextran sulfate and formamide (10% formamide, 10% dextran sulfate, 2×SSC, 0.25% Triton-X, 10% DMSO) or without those reagents (2×SSC, 0.25% Triton-X, 10% DMSO) to the sample in a well of an 8-well chamber. Next, the secondary probe and circularizer oligonucleotide mixture (containing 10 secondary probes) was, added, a tertiary probe mixture (containing 10 tertiary probes) was added, the sample was mixed using a pipette tip, a pre-mixed copper sulfate and BTTAA mixture was added, freshly-dissolved ascorbic acid was added, and again the sample was mixed using a pipette tip (with these reagents at approximately the same final concentrations as described above). After incubation of the one-pot mixtures at 37° C. for 30 minutes, the standard 10% wash buffer and 30% wash buffer washes were continued. In parallel, and with the same batches of reagents, clampFISH 2.0 was performed in the standard manner to round 1 and to round 4 as a positive control.

### Readout Cycle Steps

The following day, either directly following amplification or the subsequent conventional RNA FISH, a readout probe cycle was performed as follows. First, samples were brought to room temperature and rinsed once with room-temperature 2×SSC. For each amplifier set (each of which corresponds to a particular gene target) to be probed, two readout probes were hybridized (with one binding the secondary and one binding to the tertiary), both coupled to the same fluorescent dye. A set of readout probes for each of four spectrally distinguishable dyes could be included in a given readout cycle. Each readout probe was hybridized at a 10 nM final concentration in 5% ethylene carbonate hybridization buffer (5% ethylene carbonate, 10% dextran sulfate, 2×SSC, 0.1% Triton-X) for 20 minutes at room temperature. The solution was then aspirated, washed 1×1 minute with 2×SSC with Triton-X (2×SSC, 0.1% Triton-X), 1 minute with 2×SSC buffer, 5 minutes with 2×SSC with 50 ng/mL DAPI, then replaced with 2×SSC before imaging.

After imaging a given readout cycle, the readout probes were stripped off by incubating 2×5 minutes at 37° C. with 30% wash buffer pre-warmed to 37° C., then 2×SSC was added before starting another readout cycle. If the post-strip sample was imaged, incubation was done for 5 minutes with 2×SSC with 50 ng/mL DAPI, and the solution was replaced with 2×SSC before imaging.

For the conventional single-molecule RNA FISH comparison experiment, after stripping the readout probes conventional single-molecule RNA FISH was performed, as described above, but instead with probes for AXL, EGFR, or DDX58 without any additional readout probes.

### Imaging

For imaging a Nikon Ti-E inverted microscope equipped with an ORCA-Flash4.0 V3 sCMOS camera (Hamamatsu, C13440-20CU), a SOLA SE U-nIR light engine (Lumencor), and a Nikon Perfect Focus System. 60×(1.4NA) Plan-Apo λ (Nikon, MRD01605), 20×(0.75NA) Plan-Apo λ (Nikon, MRD00205), and 10×(0.45NA) Plan-Apo λ (Nikon, MRD00105) objective and filter sets for DAPI, Atto 488, Cy3, Alexa Fluor 594, and Atto 647N were used. All 60× images were taken using 2×2 camera binning, while 20× and 10× images used 1×1 binning.

### Image Analysis

All scripts used are all publicly accessible in a Dropbox folder (dropbox folder, which use functions from rajlabimagetools (github.com/arjunrajlaboratory/rajlabimagetools) and Dentist2 (github.com/arjunrajlaboratory/dentist2/tree/clamp2paper) repositories for spot processing and thresholding.

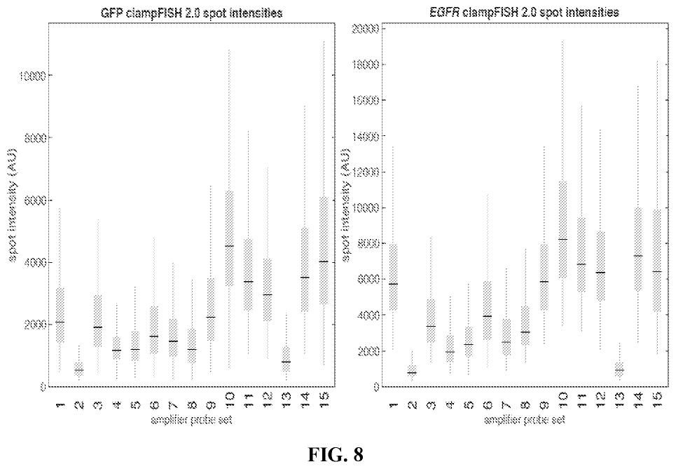

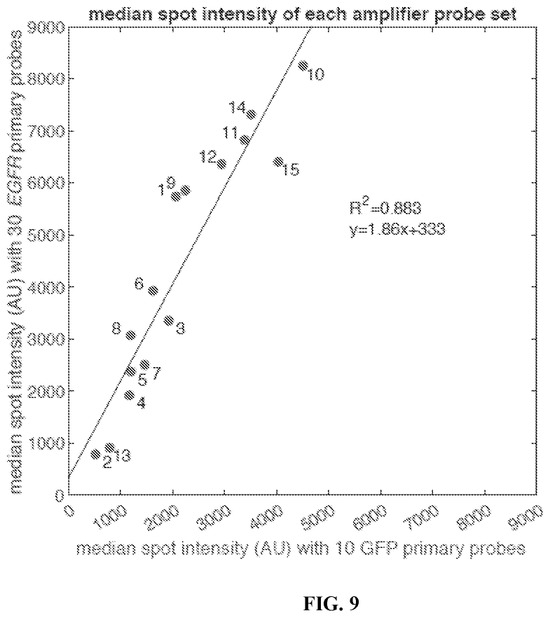

For the amplifier screen experiment, the cells were segmented in rajlabimagetools, minimum spot intensity thresholds were manually selected for conventional single-molecule RNA FISH, and the spots were counted above this threshold from a 60× magnification z-stack for each cell. For cells in which this count was 20 or greater, an equivalent number of the highest-intensity clampFISH 2.0 spots were taken from that cell and used this list of clampFISH 2.0 spot intensities for plotting in FIG. 8 and for calculation of the median intensity in FIG. 9.

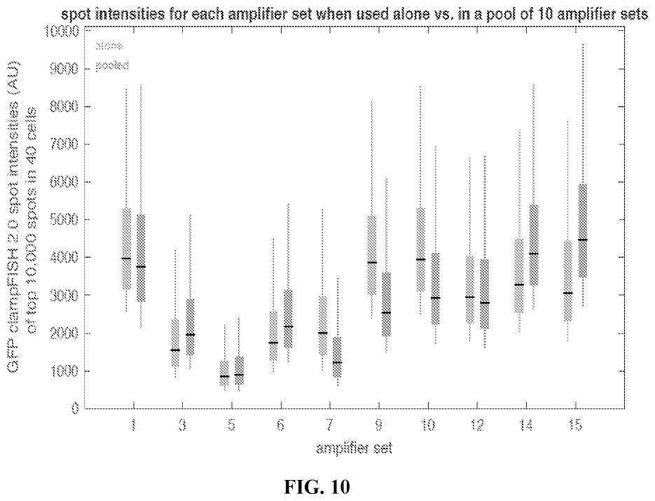

For the pooled amplification experiment (FIG. 10), in order to quantify the typical spot intensity rajlabimagetools were used to extract the 10,000 highest-intensity GFP clampFISH 2.0 spots from 60× z-stacks of 40 segmented cells per condition (an average of 250 spots per cell). The highest-intensity spots were chosen to eliminate potential biases associated with manually chosen thresholds.

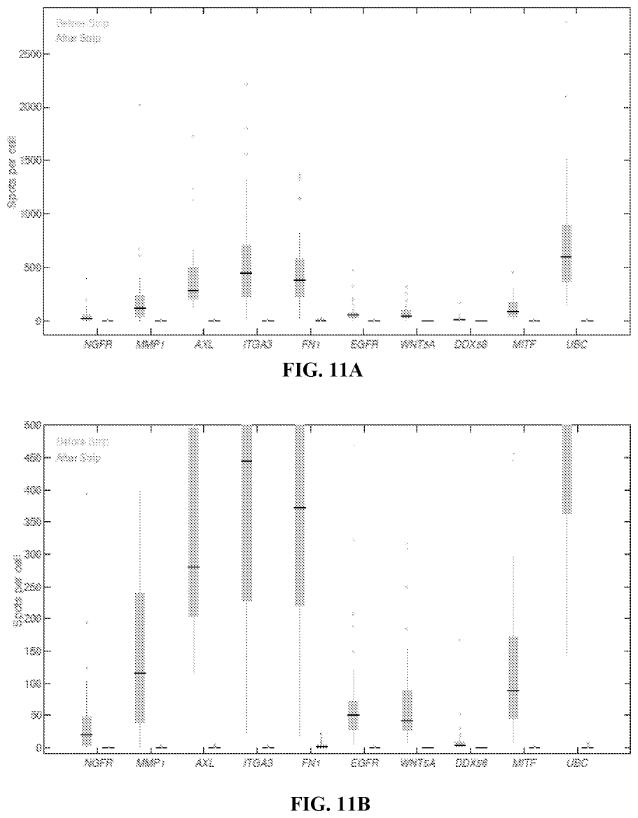

For the readout probe stripping/removing experiment (FIGS. 11A-11B), 39-48 cells were segmented in the before-stripping 20× images, gene-specific clampFISH 2.0 spot intensity thresholds were chosen, same segmentations were aligned the to the post-stripping images, and spot counts were from the post-stripping images.

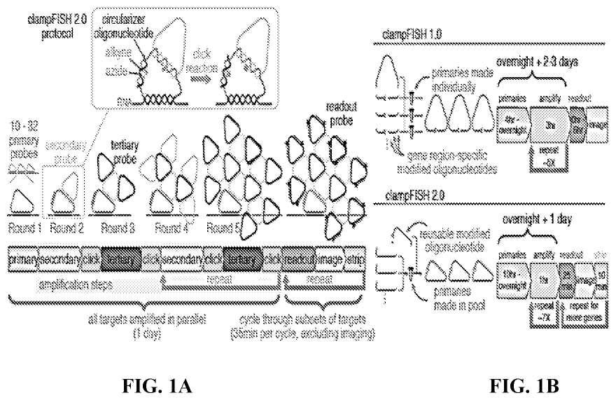

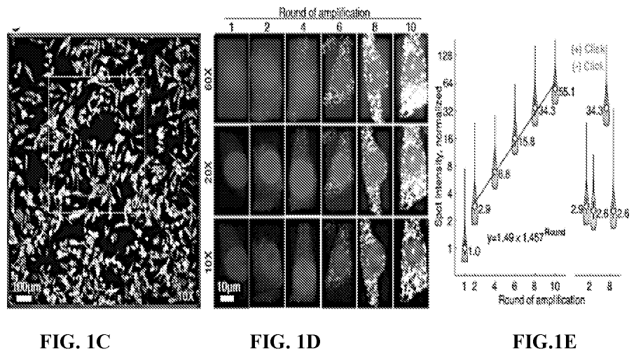

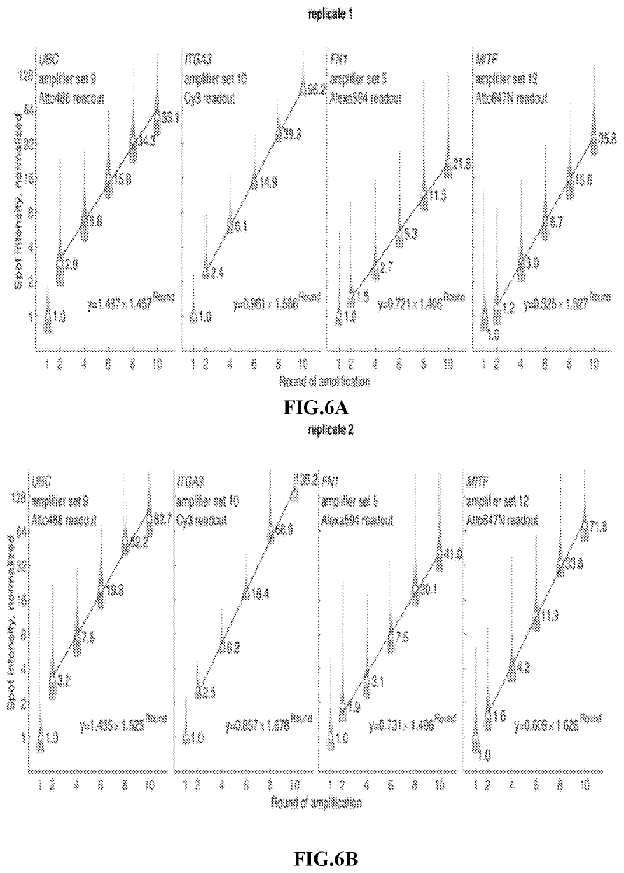

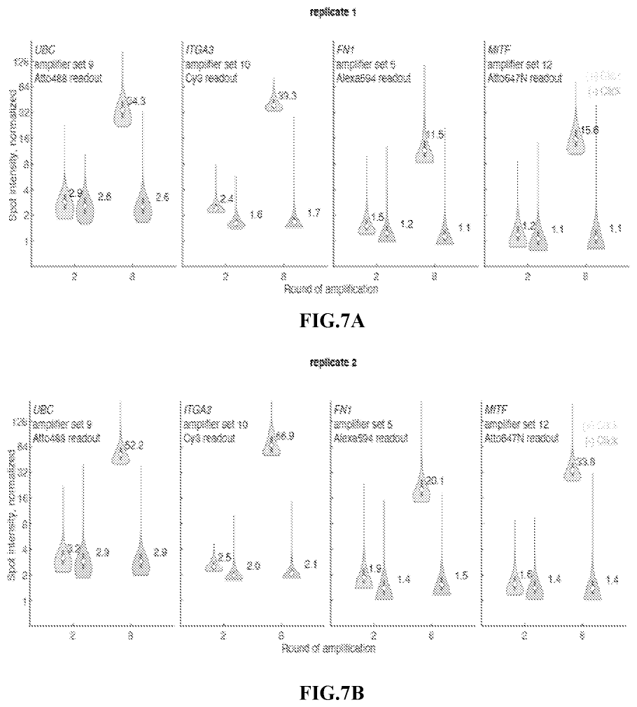

For the amplification characterization experiment, Cellpose (Stringer et al. 2021) was used to automatically segment cells using cellular background fluorescence in the YFP channel (with the DAPI channel also included as a Cellpose input), and small or large cells were excluded abnormally. For each of the 4 probed genes rajlabimagetools were used to extract the top N spots from each round of amplification, where: N=(number of cells) k, and k is the assumed average number of spots per cell (k=120, 1, 20, and 80 spots/cell for UBC, ITGA3, FN1, and MITF, respectively). To avoid saturating the camera's photon-collecting capacity at higher rounds of amplification, spots were extracted from longer exposure times on amplification rounds 1, 2, and 4 (1000, 1000, 500, and 500 milliseconds for each gene, respectively) and shorter exposure times on amplification rounds 6, 8, and 10 (all were 100 milliseconds), and these intensities were scaled by the ratio of median spot intensities between the two exposure times at round 6. For all no-click conditions, the longer exposure times to extract spot intensities were used. The data were then normalized by dividing all intensity values by the median value from round 1, using these in FIG. 1E, FIG. 6, and FIG. 7). Coefficients for the displayed exponential curve fit were calculated using a least-squares linear regression of log 2-transformed median intensity values from rounds 2, 4, 6, 8, and 10.

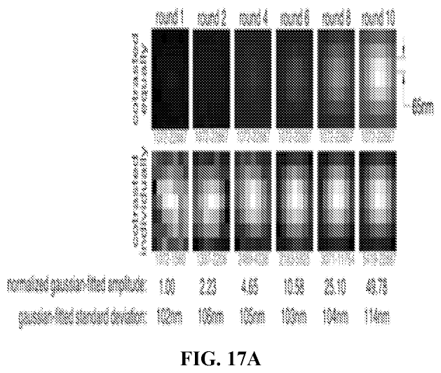

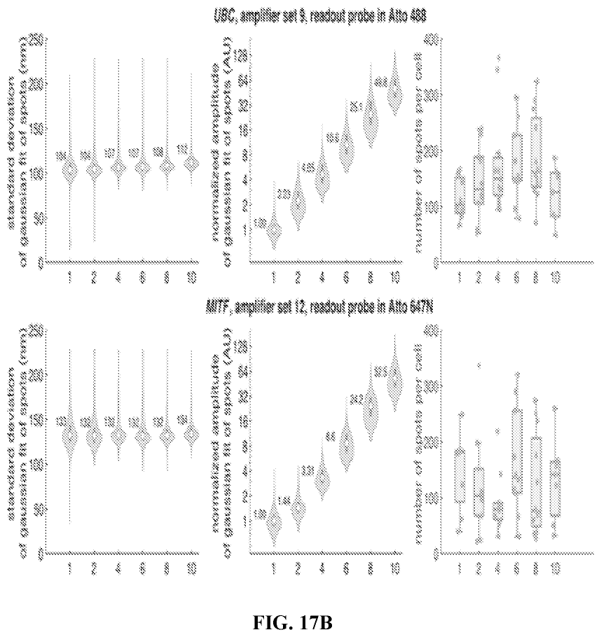

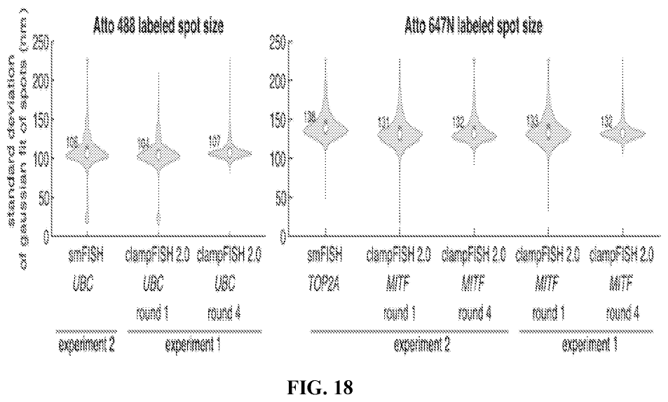

To generate plots where spot size is depicted (FIGS. 17A, 17B and 18) stacks with 15 z-planes at 0.2 μm spacing using a 100× objective (1×1 camera binning, 65 nm width per pixel) were imaged, 7-12 cells were segmented, and minimum spot intensity thresholds manually selected, where a single uniform threshold was chosen for a given clampFISH 2.0 condition whereas conventional single-molecule RNA FISH thresholds were chosen for each cell individually. A least-squares fit of the above-threshold spots at their maximum-intensity z-plane to a 2D gaussian distribution with an allowable standard deviation between 0 and 227.5 nm (0 to 3.5 pixels) was performed. To calculate a median full width at half maximum spot size, the median standard deviation of the gaussian fit was multiplied by 2.355

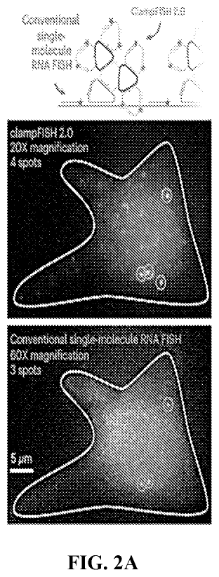

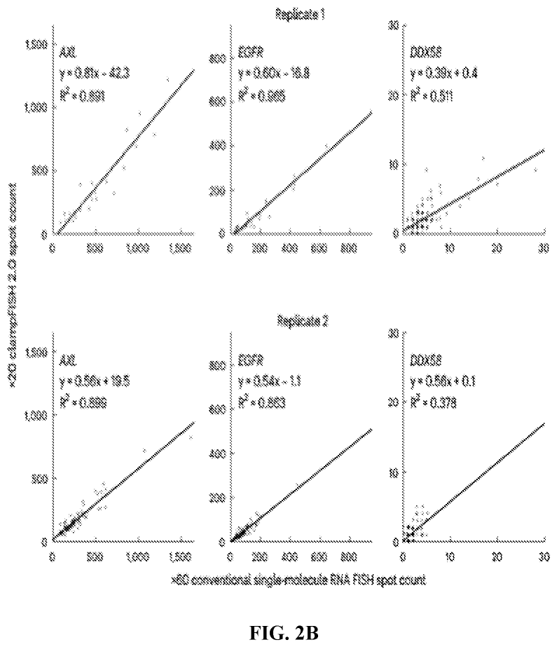

For the conventional single-molecule RNA FISH comparison experiment (FIG. 2A-2B), cells were manually segmented from 60× images using rajlabimagetools, minimum spot intensity thresholds were manually selected for the conventional single-molecule RNA FISH data for each cell individually and counted spots in each cell from 11 z-planes at 0.5 μm spacing. These segmentations were scaled and aligned to the 20× and 10× images, and clampFISH 2.0 spots exceeding a gene-specific threshold for 20×(3 z-planes at 1 μm spacing) and 10×(3 z-planes at 2 μm spacing) images were extracted. To calculate the detection efficiency for a given gene, the number of clampFISH 2.0 spots detected across all cells at 20× magnification were divided by the number of conventional single-molecule RNA FISH spots detected across all cells at 60× magnification, finding detection efficiencies of (format: replicate 1, replicate 2): AXL (73%, 63%), EGFR (49%, 53%), and DDX58 (49%, 65%). To quantify sensitivity and specificity on the lowly-expressed gene DDX58, cells with 3 or more spots were denoted as ‘DDX58 high’ and with 2 or fewer spots as ‘DDX58 low’ and did so using conventional single-molecule RNA FISH at 60× magnification (the gold standard) and using clampFISH 2.0 at 20× magnification. In two biological replicates (different passages of WM989 A6-G3 cells), it was found that clampFISH 2.0 at 20× magnification could identify ‘DDX58 low’ cells with a specificity of 97% (32/33 cells, replicate 1) and 99% (86/87 cells, replicate 2) and ‘DDX58 high’ cells with a sensitivity of 41% (35/86 cells, replicate 1) and 53% (10/19 cells, replicate 2).

For the high-throughput profiling experiment, the tiled scans were stitched and registered from multiple imaging cycles at 20× magnification using the custom pixyDuck repository and then divided the scan into smaller subregions. Imaged were 5 wells (replicate 1) and 1 well (replicate 2) of WM989 A6-G3 cells, dividing those scans into 10×10 subregions, and 1 well (replicates 1 and 2) of WM989 A6-G3 RC4 cells, dividing those scans into 6×6 subregions. Dentist2 was used to choose spot intensity thresholds, extract spots, and then assign those spots to cellular segmentations generated by Cellpose based on cellular background fluorescence (eg. autofluorescence) in the YFP channel (using the diameter parameter of 90 pixels for WM989 A6-G3 cells and 350 pixels for WM989 A6-G3 RC4 cells). The housekeeping gene UBC, for which a readout probe was hybridized on every readout cycle, was used for the following quality control steps. First, only subregions where there was an average of at least 25 UBC spots per cell for all readout cycles were kept (it was observed that near the edges of the wells, fewer spots above the chosen thresholds were detected, presumably because the coverslip used to spread out all probe-containing solutions were smaller than the full well). Only cells were taken where, for all readout cycles, the UBC spot count was: at least 4, at least 0.025/μm2×cell area, always within 50% of the median count from all readout cycles. Out of the initial 1,297,062 (replicate 1) and 253,662 (replicate 2) WM989 A6-G3 cells segmented, 722,298 (replicate 1) and 234,410 (replicate 2) cells passed all quality control metrics and were included in downstream analyses.

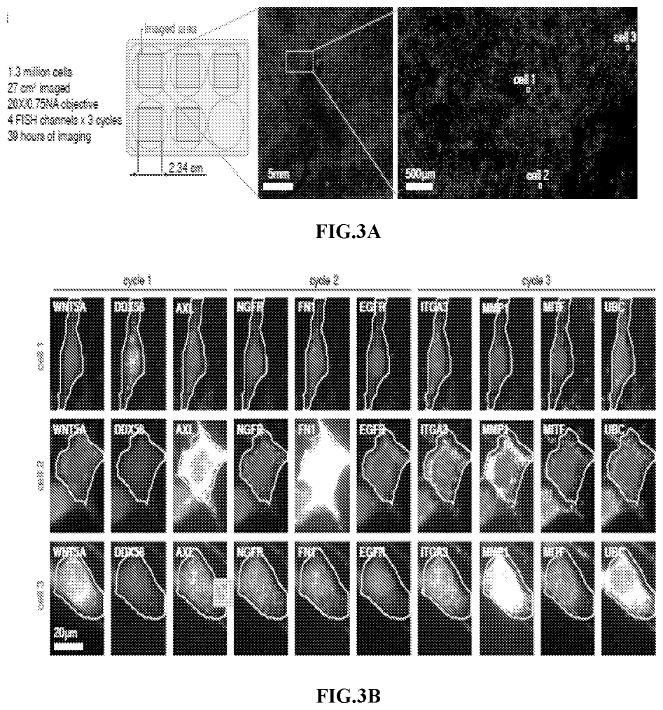

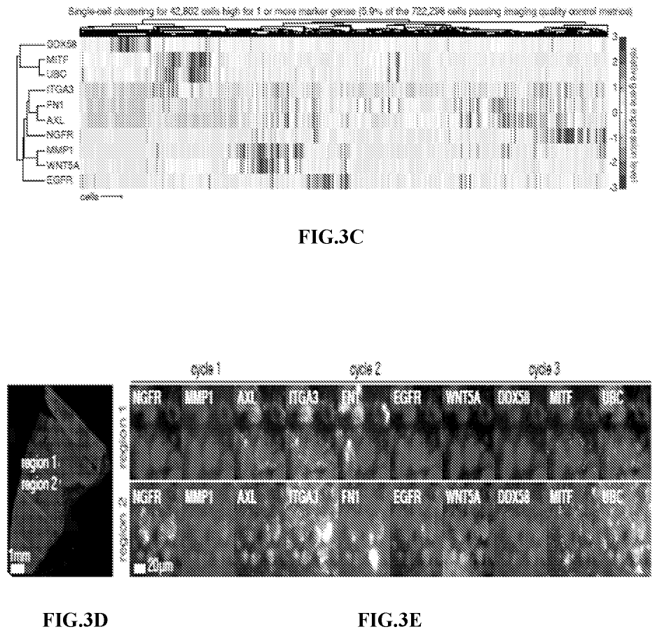

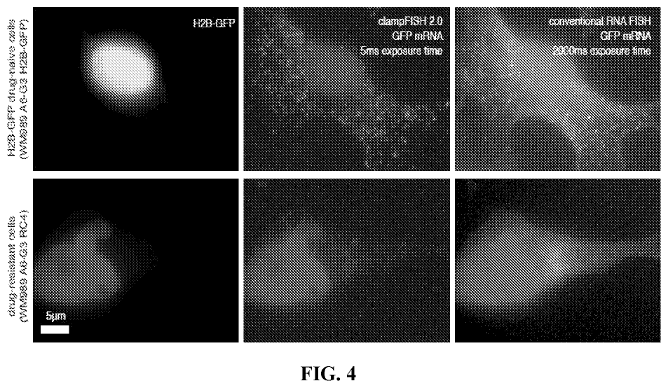

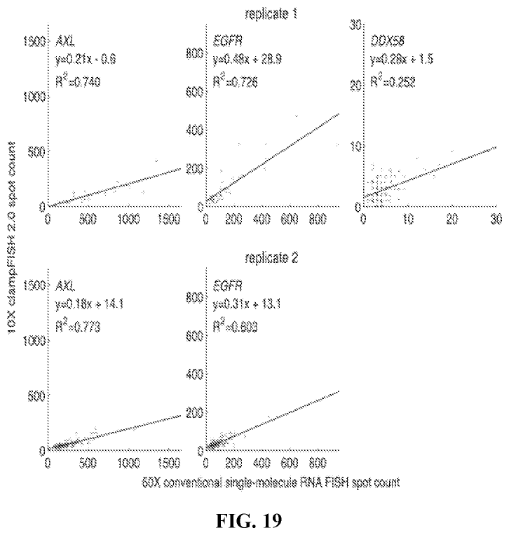

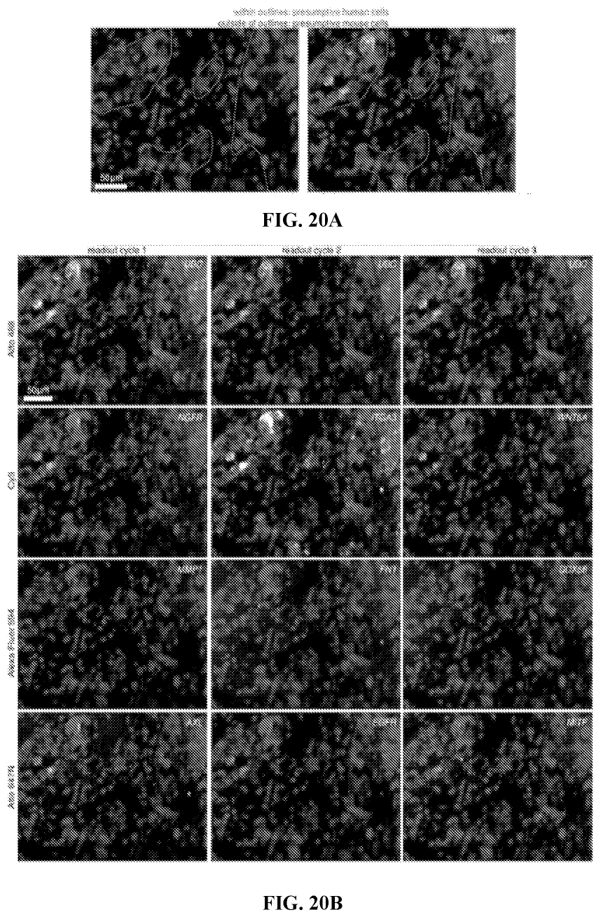

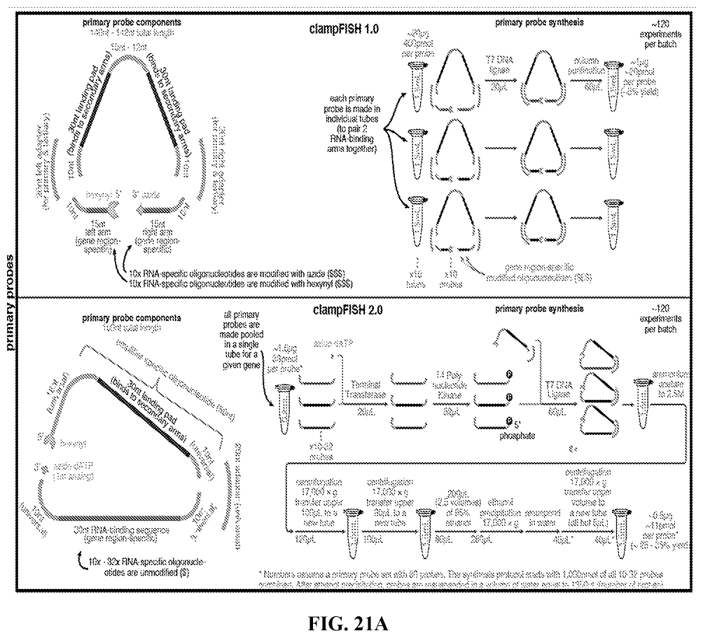

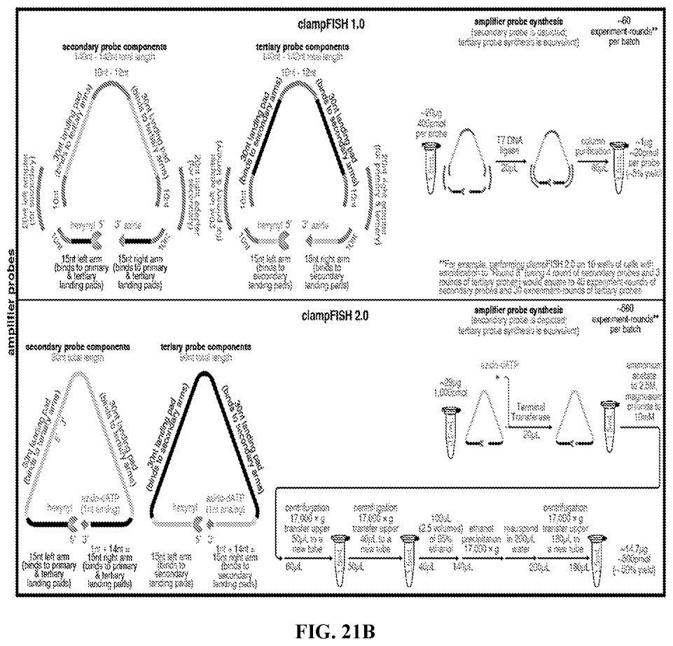

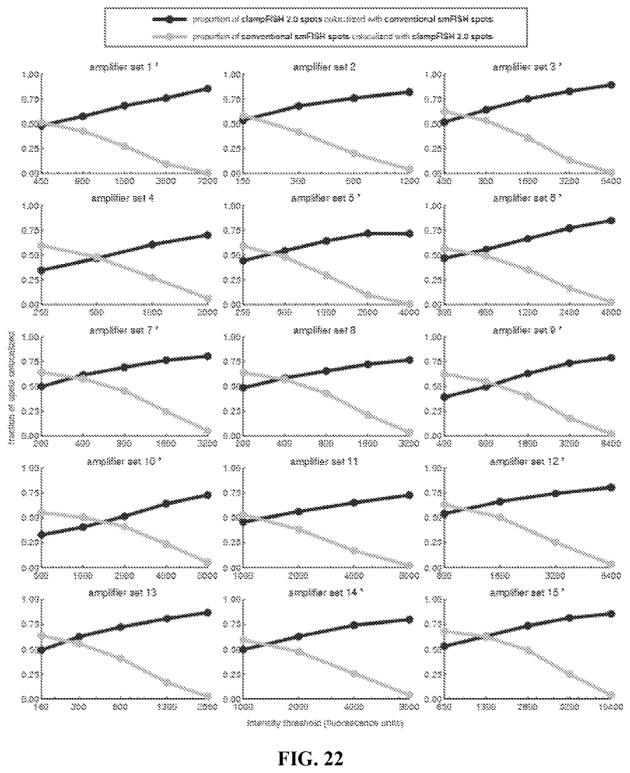

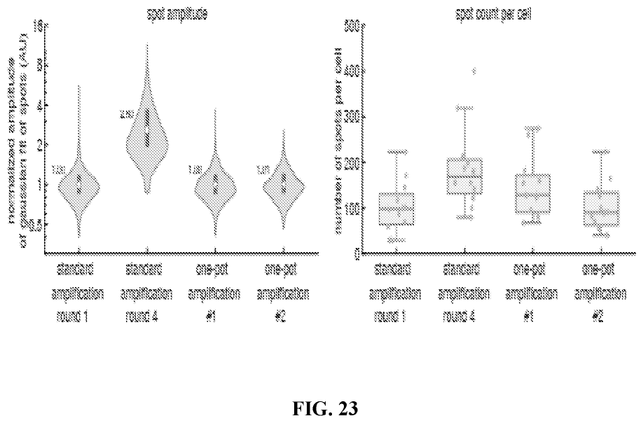

To analyze only cells expressing high levels of one or more of 8 marker genes, chosen for each gene were the following minimum spot count thresholds (format: minimum spot count to be considered high-expressing, percentage of cells high-expressing in replicate 1): WNT5A (>=15, 0.59%), DDX58 (>=10, 0.56%), AXL (>=25, 3.56%), NGFR (>=30, 1.07%), FN1 (>=100, 2.79%), EGFR (>=5, 1.40%), ITGA3 (>=50, 2.31%), MMP1 (>=40, 1.48%). For the 5.93% of cells (42,802 out of 722,298) in replicate 1 and the 10.5% of cells (24,685 out of 234,410) in replicate 2 expressing high levels of one or more marker genes, MATLAB's cluster gram function was used to perform hierarchical clustering using all 10 genes' normalized spot counts (replicate 1: FIG. 3C; replicate 2: FIG. 19), where each gene's spot counts were transformed such that the mean is 0 and the standard deviation is 1.

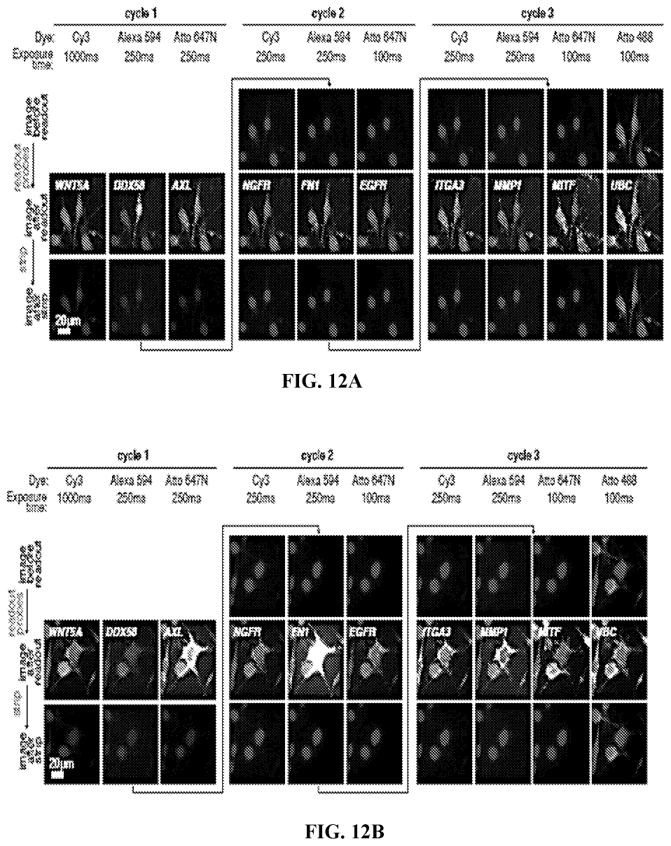

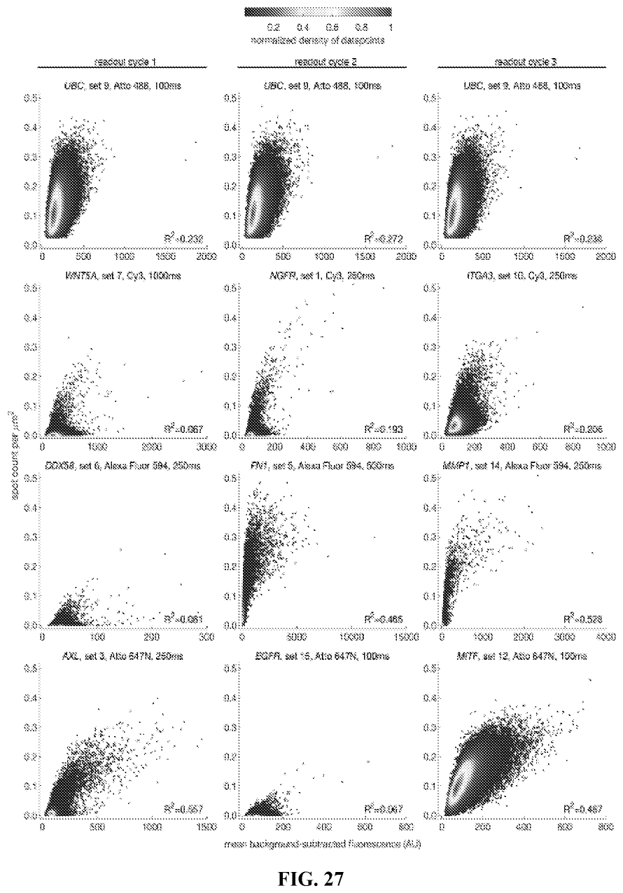

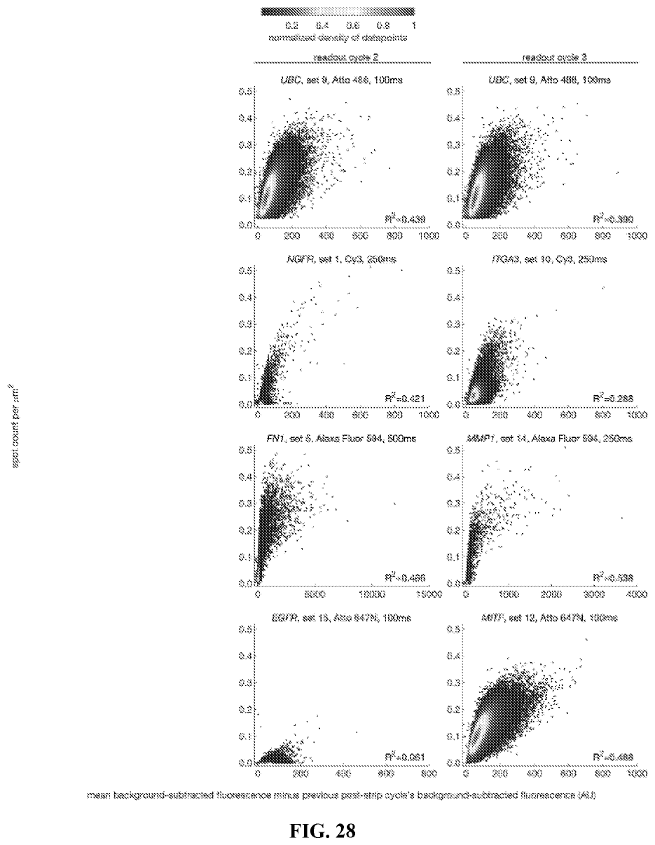

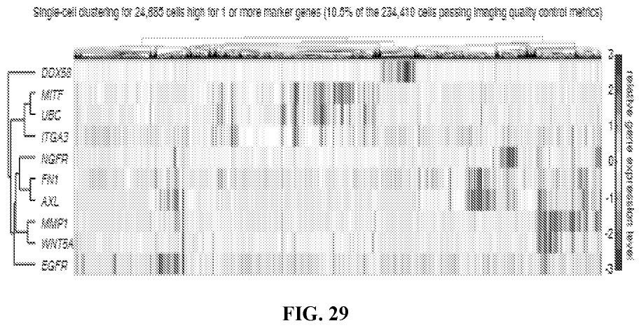

For FIGS. 12A-12B, 13, 27, 28, the same pipeline was ran on a smaller imaged area in Well A1 that, in addition to the three readout cycles included previously, also included a re-imaging of readout cycle 1 and readout cycles 4 and 5 (both of which re-used the same readout probes from readout cycle 1). To define spots, a single minimum spot intensity threshold was chosen for each gene on each round. Thresholds for readout cycle 4 images were made the same as those in cycle 1. For readout cycle 5 (performed after storing the sample at 4° C. for 4 months on replicate 1 only), the thresholds were increased by 67% to 83% (the cycle 5 signal presumably appeared brighter due to changes in the microscope's optical path, i.e. greater sample illumination or increased transmission to the sensor). To calculate each cell's mean background levels for FIGS. 27 and 28, a background image was generated for each gene and imaging cycle by selecting 100 random image tiles with clampFISH 2.0 signal, generating an image using the 5th percentile of the 100 values for each pixel position, performing gaussian-smoothing on this image, and then averaging these smoothed values in the cellular segmentation. Mean background-subtracted fluorescent intensity was calculated by averaging the pixel values in the cellular segmentation and subtracting the mean background level. To further correct for background contributed by autofluorescence and any residual fluorescence from previous readout cycles, in FIG. 28 from the mean background-subtracted fluorescent intensity of the clampFISH 2.0 signal subtracted is the mean background-subtracted fluorescent intensity derived from images taken after the previous readout probes have been stripped off but before the new clampFISH 2.0 readout probes are introduced.

### RNA Sequencing

Bulk RNA sequencing was performed as described in (Goyal et al. 2021). Standard bulk paired-end (37:8:8:38) RNA sequencing was conducted using RNeasy Micro (Qiagen, 74004) for RNA extraction, NEBNext Poly(A) mRNA Magnetic Isolation Module (NEB E7490L), NEBNext Ultra II RNA Library Prep Kit for Illumina (NEB, E7770L), NEBNext Multiplex Oligos for Illumina (Dual Index Primers Set 1) oligos (NEB, E7600S), and an Illumina NextSeq 550 75 cycle high-output kit (Illumina, 20024906), as previously described (Mellis et al., 2021; Shaffer et al., 2017). Prior to extraction and library preparation, the samples were randomized to avoid any experimental and human biases. The RNA-seq reads were aligned to the human genome (hg19) with STAR v2.5.2a and uniquely mapping reads were counted with HTSeq v0.6.1 (Dobin et al., 2013; Mellis et al., 2021; Shaffer et al., 2017) and outputs count matrix. The counts matrix was used to obtain tpm and other normalized values for each gene using scripts provided at: (github.com/arjunrajlaboratory/RajLabSeqTools/tree/master/LocalComputerScripts).

### Probe Re-Design and Protocol Optimization

ClampFISH 1.0's primary probes were assembled with two gene-specific oligonucleotides that each required chemical modification, substantially adding to the method's cost. It was therefore asked whether it was possible invert the primary probes' orientation, such that the gene-specific RNA-binding oligonucleotide components could remain unmodified, and therefore cheaper, while incorporating the click chemistry modifications into a reusable, gene-independent oligonucleotide. In this scheme, a separate ‘circularizer oligo’ was also add to help ligate the primary probe, while keeping the orientation of the secondary and tertiary probes unchanged (FIG. 1A). This new primary probe design had the additional benefit of permitting larger-scale probe synthesis, since all of a gene's primary probes could be ligated in a single pooled reaction (FIG. 1B). As a potential downside, the benefits of the new design could, in principle, come at the expense of specificity: the lack of a proximity ligation mediated by the target RNA molecule could allow for more non-specific probe self-ligation, because the proximity ligation is typically thought to increase specificity. Along with re-designing the primary probes, the length of the secondary and tertiary probes (collectively referred to as ‘amplifier probes’) was also shortened, such that they can be made from a single commercially-produced oligonucleotide, thus simplifying their formerly 3-part synthesis.

In addition to its high cost, the clampFISH 1.0 protocol was time-consuming, in large part because each round of amplification required approximately 3 hours. For example, the amplification protocol would require 2 days with 4-5 rounds of amplification, or 3 days for 6-8 rounds of amplification. Taking note of reports that reduced nucleic acid secondary structure permits faster hybridization (Gao, Wolf, and Georgiadis 2006; Zhang et al. 2014), it was reasoned that it would be possible to reduce the 2 hour amplifier hybridization time by using amplifier probes design to have a low predicted secondary structure, an approach that's also been used for branching amplification (Xia et al. 2019). With these new probe designs and additional optimization of the wash steps, click reaction, and buffer compositions, the time for a round of amplification was reduced from 3 hours to just 1 hour, which includes a 30-minute amplifier hybridization. This 3-fold speed improvement in amplification allows the full protocol, up to readout probe hybridization and imaging, to be performed with an overnight primary incubation (10 hr+) and about 8 hours the next day (FIG. 1B).

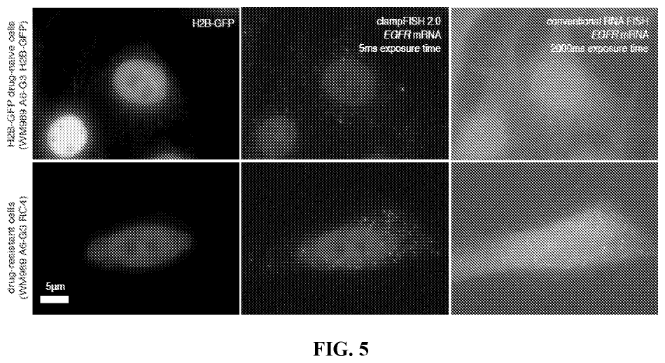

It was queried whether this updated scheme would still produce specific, amplified RNA signal, as did the original clampFISH 1.0. Primary probes for each of two separate mRNA targets (GFP mRNA, 10 probes; and EGFR mRNA, 30 probes) were made and their performance was tested on a mixture of two cell lines known to express different RNAs: an H2B-GFP WM989 line, expressing the GFP sequence as mRNA, and a WM989 line grown in drug-containing media that we have shown to express high levels of EGFR mRNA (Shaffer et al. 2017; Emert et al. 2021; Goyal et al. 2021). Bright, amplified spots were observed for the mRNAs specifically in the cells that were expected to express them (FIGS. 4 and 5), confirming the method's specificity despite the new primary probes lacking an RNA-splinted proximity ligation.

It was next sought to determine whether clampFISH 2.0 could exponentially amplify signal to a level that is detectable with lower-powered (20×/0.75NA and 10×/0.45NA) air objective lenses. The clampFISH 2.0 protocol was ran to varying stopping points: 1 round (primaries), 2 rounds (primaries and secondaries), 4 rounds (primaries, secondaries, tertiaries, and secondaries again), 6 rounds, 8 rounds, and 10 rounds, and readout probes were hybridized to these scaffolds. Using low-powered magnification with large fields of view, the spots could be reliable detected after amplification, thus demonstrating clampFISH 2.0's capacity for high-throughput RNA detection (FIGS. 1C-1D). Furthermore, an exponential rate of amplification, measured to be 1.406 to 1.527-fold per round (FIG. 1E, FIG. 6), was observed implying the amplifier binding efficiency is 70-76% of the theoretical doubling of intensity per round. This exponential rate of growth did not appreciably slow down, even at the maximum number of rounds tested (round 10), suggesting that an even brighter signal could be achievable with additional amplification.

In order to achieve a higher degree of multiplexing, a number of sets of amplifier probes that had high gain and low off-target activity were needed. 15 amplifier probe sets were thus screened, each used with primary probes targeting GFP mRNA or EGFR mRNA. Of these, 10 sets of amplifier probes (1, 3, 5, 6, 7, 9, 10, 12, 14, and 15) with high gain and low off-target activity (amplifier set 11 was excluded based on its high number of off-target spots) were chosen. It was observed that an amplifier probe set's gain for one RNA target strongly correlated with its gain on the other RNA target, indicating that amplifiers can be used in a modular fashion with any set of primary probes without substantial primary-probe-specific effects on performance (FIGS. 8 and 9). It was also confirmed that amplifiers do not cross-react with one another by showing that the spot intensities were equivalent when amplifier sets were used individually versus when they were used in a pooled mixture of other amplifiers (FIG. 10).

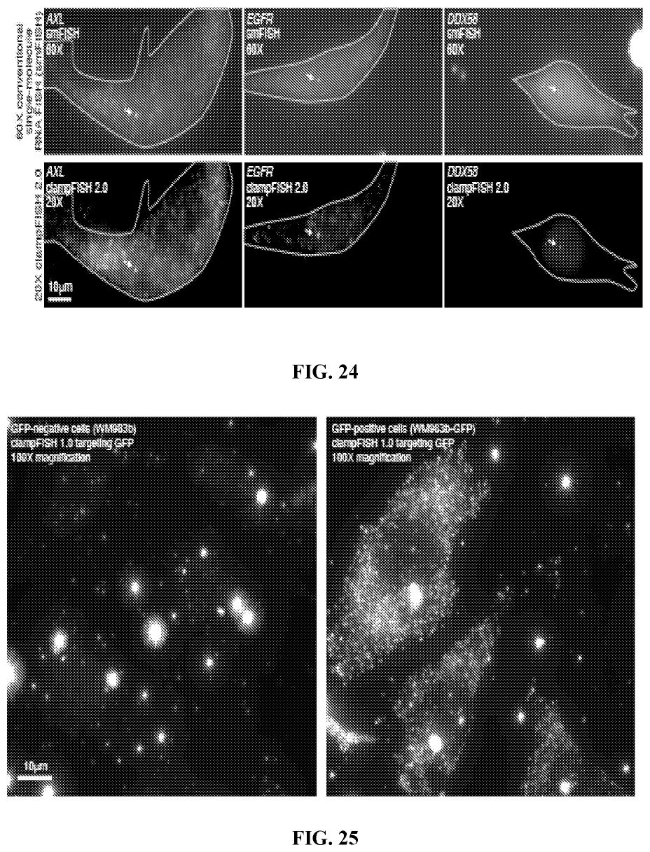

Given the method's capacity for fast, flexible multiplexed RNA detection, the method's quantitative accuracy when used at low magnification, a capability useful for high-throughput imaging, was characterized. ClampFISH 2.0 was performed to round 8 (one round of primary probes and seven rounds of amplifier probes) targeting three human mRNAs (EGFR, AXL and DDX58) with a range of expression levels. After the clampFISH 2.0 protocol, hybridized were conventional, unamplified single-molecule RNA FISH probes as a gold standard, which were designed to bind to non-overlapping sites on the same mRNA. It was possible to observe many of the same spots with clampFISH 2.0 at ×20 magnification that were seen using conventional single-molecule RNA FISH at ×60 high magnification, confirming the method's high sensitivity and specificity (FIG. 2A). In addition to the ninefold larger field of view and greater depth of field offered by ×20 magnification compared with ×60 magnification, clampFISH 2.0 spots were detected at ×20 using shorter exposure times (100 ms for EGFR, 250 ms for AXL and 500 ms for DDX58) in comparison with the 2 s exposure time used with conventional single-molecule RNA FISH. On comparison of the spot counts for multiple targets between clampFISH 2.0 at ×20 magnification and conventional single-molecule RNA FISH at ×60 magnification, clampFISH 2.0 detection efficiency between 49% and 73% (see Methods) and a high correlation in spot counts (FIG. 2B) were observed, demonstrating that clampFISH 2.0 can be used as a higher-throughput replacement for conventional single-molecule RNA FISH. Even for a target (DDX58) expressed at low levels in a subset of cells, it was possible to accurately identify cells with three or more RNAs (41-53% sensitivity, 97-99% specificity), thus supporting the ability of clampFISH 2.0 to reliably quantify even low-expression genes at ×20 magnification. No particular decrease in clampFISH 2.0 detection efficiency was observed with increasing conventional single-molecule RNA FISH counts (that is, AXL, EGFR), suggesting that under-counting of spots at ×20 due to optical crowding is minimal in this range of expression levels. Comparing spot counts from clampFISH 2.0 at ×10 magnification with conventional single-molecule RNA FISH at ×60 magnification, a reduction was seen in the correlation strength (for example, when targeting AXL an R2 of 0.740-0.773 at ×10 magnification was observed, versus an R2 of 0.891-0.899 for ×20 magnification; FIG. 2B and FIG. 19), suggesting that more accurate quantification using ×10 magnification may require additional rounds of amplification beyond round 8. Although the majority of clampFISH 2.0 spots lie in the cytoplasm, as expected, spots were also detected in the nucleus (FIG. 3B) and, unlike with clampFISH 1.0, at transcription sites (FIGS. 24 and 25), a feature of clampFISH 2.0 that enables high-throughput analyses involving RNA localization.

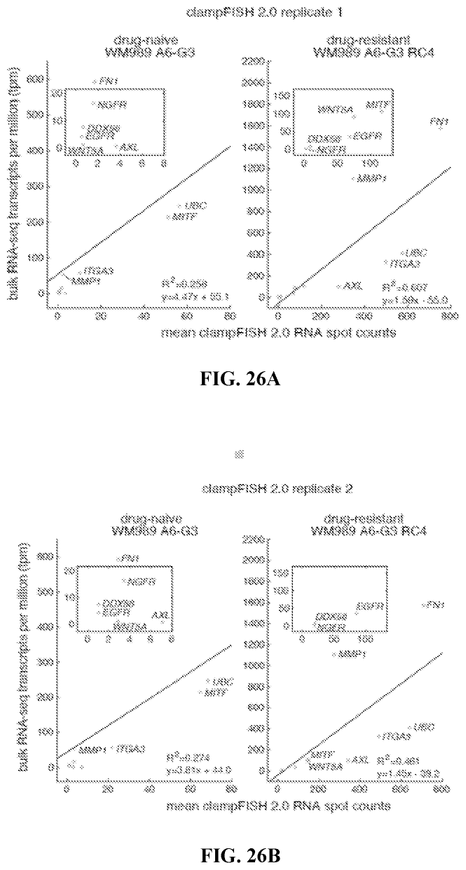

As an additional measure of the quantitative performance of clampFISH 2.0, the average clampFISH 2.0 spot count for 10 human gene targets were compared with their relative abundance (transcripts per million) as detected by bulk RNA sequencing and found a moderate correlations in two melanoma cell lines (R2 between 0.256 and 0.607; FIG. 26A-26B). It was observed that FN1 and MMP1, both of which have a lower mean clampFISH 2.0 spot count than would be expected from the trend of the remaining genes, are expressed at particularly high levels in a subset of cells (FIG. 3B), suggesting that optical crowding at ×20 magnification may contribute to their under-counting by clampFISH 2.0. Correlations between the ×20 magnification spot count and mean fluorescence intensity suggested that for cells with particularly high RNA copy numbers, the cell's mean fluorescence intensity might be useful to correct for under-counting due to optical crowding, even though mean fluorescence intensity is not an accurate proxy for spot counts with typical RNA copy numbers (FIGS. 27 and 28).

### Example 1: Iterative Hybridization Enables Profiling of Over One Million Cells

A crucial advantage of clampFISH 2.0 is its potential for rapid multiplexing through iterative hybridization of readout probes. Iterative hybridization refers to schemes for multiplexing beyond the spectral capabilities of conventional fluorescence microscopes (Lubeck et al. 2014). The basic idea is to detect RNA FISH signal from a small number (typically 3-4) of RNA targets using spectrally distinct fluorophores for each target. To measure RNA FISH signal from more targets in the same cells, the signal from the current set of targets is removed and then another round of hybridization to the next set of targets is performed, enabling detection of another set of RNA species. clampFISH 2.0 in principle is ideally suited for such iterative schemes because all the scaffolds can be generated at once before any readout steps, and the short readout probes could be stripped and reprobed very rapidly.

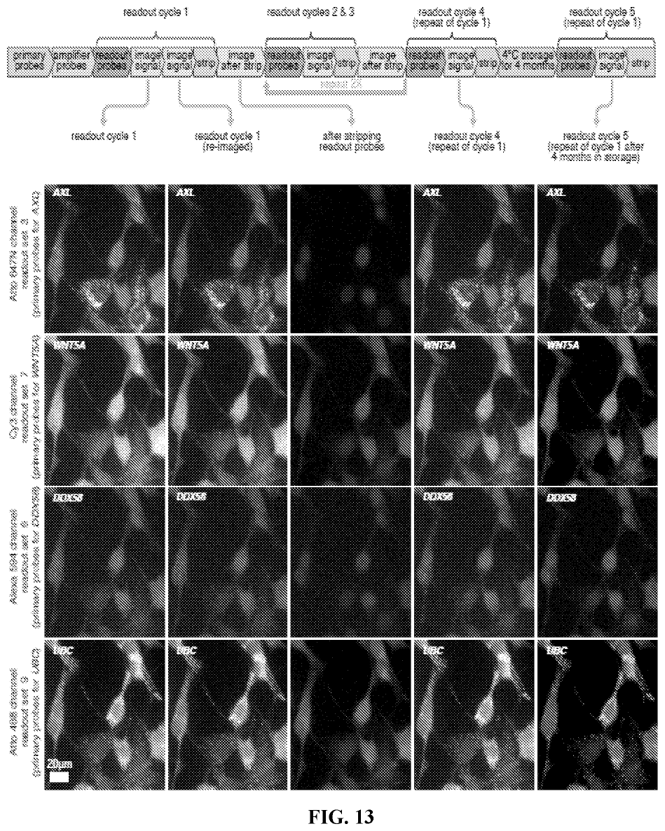

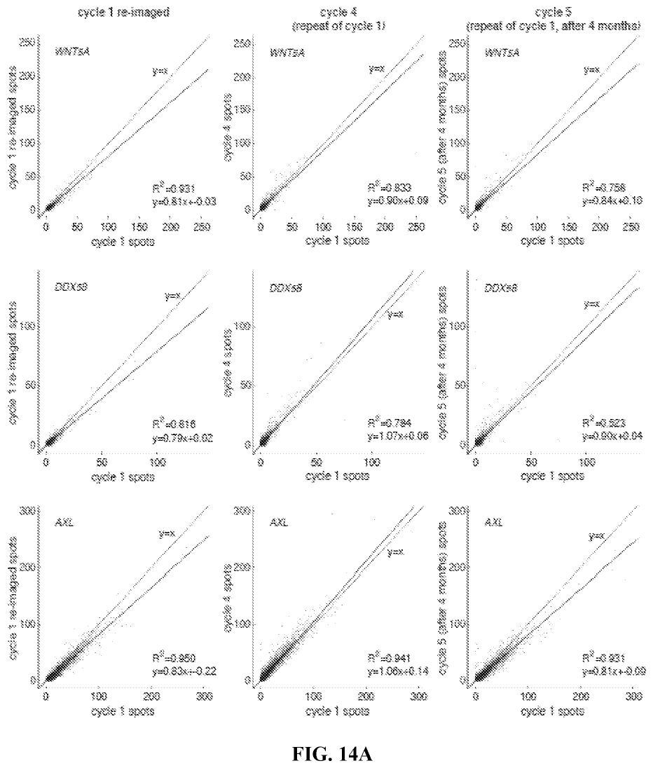

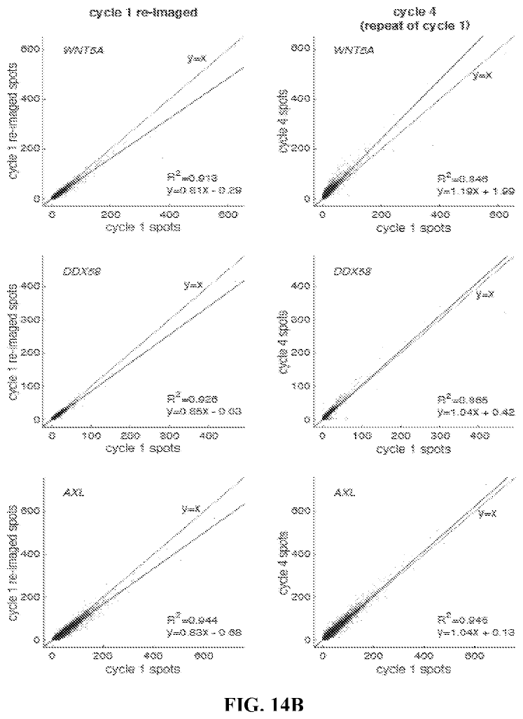

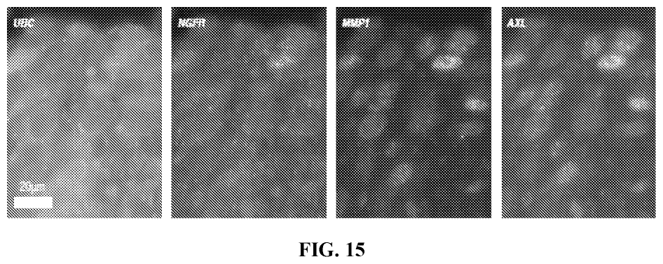

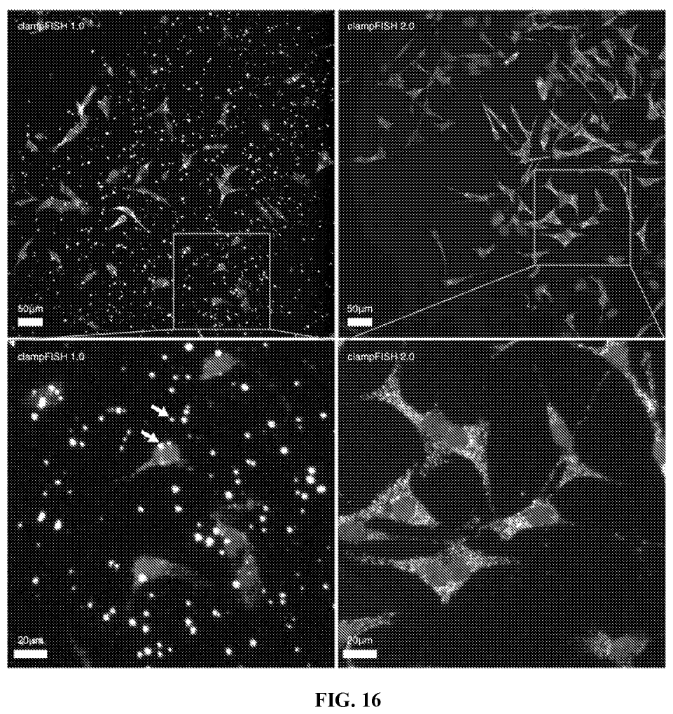

An important first step for iterative hybridization is the ability to remove the fluorescent signal from the sample after imaging. Thus, it was first tested whether the readout probes could be reliably stripped from their scaffolds with a simple high-stringency wash. The mRNA were probed from 10 genes, each with its own primary probe set with one of ten amplifier-specific sequences (pairing gene 1 with amplifier set 1, gene 2 with amplifier set 2, and so on), and generated scaffolds by amplifying to round 8. With these scaffolds generated in three separate wells, 4 spectrally separable sets of readout probes (coupled to Atto488, Cy3, Alexa Fluor 594, or Atto 647N) were then hybridized, each binding to a specific amplifier set, thus visualizing four genes simultaneously per well (10 genes total, where scaffolds for 1 gene, UBC, were probed in all 3 wells). After imaging these spots, the readout probes were stripped off with 30% formamide in 2×SSC, re-imaged the samples, and noticed nearly all spots were removed (FIG. 11). Since the clampFISH 2.0 scaffolds are constructed of interlocking loops, the scaffolds were expected to remain stably attached to the mRNA targets despite the dissociation of the readout probes. Indeed, when the same scaffolds were re-probed after multiple rounds of readout hybridization and stripping, the same spots as in the initial readout round were detected (FIG. 13 and FIG. 14), thus demonstrating the stability of the scaffolds. In fact, even after leaving a sample refrigerated for 4 months, the same spots were detected after re-hybridizing the readout probes (FIG. 14), thus allowing flexibility in the timing of readout and imaging.

Having demonstrated the ability to strip off readout probes, it was then attempted to detect the mRNA from 10 different genes simultaneously in individual cells. Expression was tested for genes WNT5A, DDX58, AXL, NGFR, FN1, EGFR, ITGA3, MMP1, MITF, and UBC at the same time in the melanoma WM989 A6-G3 cell line (Shaffer et al. 2017) (and WM989 A6-G3 RC4 cells; see methods for details). Cells spread over 5 wells of a 6-well culture dish were imaged with 3 cycles of imaging. Each imaging cycle consisted of detection in 4 readout probe channels, with (B (probed in every cycle as a control for consistency (see methods for details). The amplified signal allowed for a typical exposure time of 250 ms with a 20×/0.75NA objective lens, allowing us to detect 10 genes in 1.3 million cells in 39 hours of imaging (FIGS. 3A and 3B), demonstrating the ability to perform multiplex gene expression analysis via iterative hybridization across a large number of individual cells.

As a demonstration of the sorts of analyses that such high-throughput multiplexed RNA quantification enabled, the co-expression of these genes was analyzed in the rare subpopulations that express them. Previous work has demonstrated that these genes express in only rare cells (1:50-1:500), and that that it is these rare cells with high expression that are the ones that survive targeted drug therapies (Shaffer et al. 2017; Emert et al. 2021; Schuh et al. 2020). Many of these genes co-express in single cells (Shaffer et al. 2017), but the precise coexpression relationships have been hard to decipher due to the rarity of the expression. It was reasoned that the much higher number of cells that were possible to image with multiplex clampFISH 2.0 (˜1.3M vs. ˜8700 for conventional single molecule RNA FISH (Shaffer et al. 2017)) would enable one to measure these relationships. Using automated cell segmentation (Stringer et al. 2021) and a spot-detection pipeline, 42,802 cells were identified with one or more marker genes positively-associated with drug resistance out of a total pool of 722,298 cells. This sample size was large enough that it allowed to observe distinct clusters of co-expression (FIG. 3C), demonstrating the sorts of analyses that are now possible with the high throughput of clampFISH 2.0.

### Example 2: clampFISH 2.0 Detects RNA in Tissue Sections

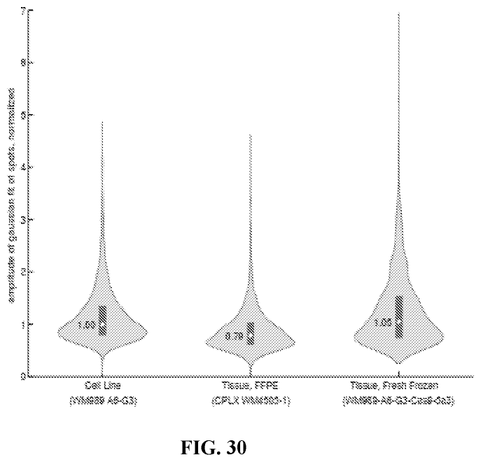

An important application of image-based gene expression detection methods is in multicellular organisms and tissues. To demonstrate that clampFISH 2.0 could work in this context as well, we used the same 10 gene panel described above in fresh frozen tumor sections that were sliced into 6 μm thick sections. These sections came from the injection of WM989-A6-G3-Cas9-5a3 cells into mice, which subsequently grew into tumors and were then treated with vemurafenib (samples first used in (Torre et al. 2021); see that paper for details). ClampFISH 2.0 signal was observed in many of the cells, including consistent UBC signal across virtually all cells, as expected. The signals observed had intensity similar to that observed in cell culture, confirming that clampFISH 2.0 was able to detect RNA in tissue sections. ClampFISH 2.0 was also performed in a formalin-fixed paraffin embedded tissue section, in which dimmer UBC clampFISH 2.0 signal was seen (FIG. 15). There were regions of the tissue section that were completely devoid of signal, perhaps due to sample degradation or other unknown factors. It was found that ITGA3 spot intensities in a fresh frozen tissue section were of a similar intensity to those in a cell line, while the spots in a FFPE tissue section were ˜20% dimmer (FIG. 30).

### Example 3

Described herein is the development of an improved version of clampFISH 2.0. Key features are the inverted probe design, which makes probe synthesis far more cost and time efficient, and the increased speed of the protocol. In particular, the efficiencies for probe synthesis are critical for multiplex applications in which one targets multiple RNA species at the same time.

One important aspect of amplified signal is that one can use lower resolution optics, in particular at lower magnification. By using a 20× (or 10×) objective, it is possible to obtain a 20-25 fold (40-75 fold) increase in throughput (number of cells imaged per unit time) as compared to conventional single molecule RNA FISH imaged using a 60× objective. These order-of-magnitude increases in throughput can enable many new applications, especially in the detection of rare cell types. It is possible that other imaging improvements may be enabled by the dramatically increased signal afforded by signal amplification.

While demonstrated herein is a straightforward iterative hybridization scheme for multiplex RNA detection, one could imagine using clampFISH 2.0 for more complex combinatorial multiplex schemes as well (Lubeck et al. 2014; Shah, Lubeck, Schwarzkopf, et al. 2016; Shah, Lubeck, Zhou, et al. 2016; Eng et al. 2019; Moffitt, Hao, Wang, et al. 2016; Moffitt, Hao, Bambah-Mukku, et al. 2016; Xia et al. 2019). Many of those schemes rely on the detection of the same RNA in a specified subset of iterative detection rounds. clampFISH 2.0 could be particularly well-suited for such schemes, because one could use combinations of readout probes in each round to detect specific RNA species with specific fluorophores. Another potential benefit of clampFISH 2.0 for such sequential barcoding schemes is the small optical size of the spots, which are generally at or near the diffraction limit. Both hybridization chain reaction and rolling-circle amplification produce spots that are larger (up to ˜1 μm) (Xia et al. 2019; Shah, Lubeck, Schwarzkopf, et al. 2016; Lee et al. 2015) than diffraction limit spots, which has can cause optical crowding-if visualizing a large number of spots, they can run together, making it difficult to discriminate neighboring spots. That makes it particularly difficult to colocalize spots through multiple rounds of hybridization and imaging. Other benefits of diffraction limited spot size is that the small size is beneficial for accurate super-resolution structural analysis by e.g. STORM or STED, and also that many analysis tools assume diffraction limited spots as input to the image. That readout probes can be re-hybridized to the same scaffolds offers flexibility in sequential encoding schemes. For example, whereas the sequential barcode is normally encoded by the library of RNA-binding probes, which cannot be modified after their construction, instead each gene might have a single associated amplifier set, where the choice of each imaging cycle's subset of readout probes would define the barcode, providing more flexibility for individual experiments to probe different gene subsets using the same primary probe library.

Another potential benefit of clampFISH 2.0 for such sequential barcoding schemes is the small optical size of the spots, ˜264 nm and ˜316 nm full width at half maximum for Atto 488- and Atto 647N-labeled readout probes, respectively. Both HCR and rolling circle amplification produce spots that are larger (up to ˜1 μm) 18,29,32 than diffraction-limited spots, which contributes to optical crowding: when visualizing a large number of spots, they can overlap, making it difficult to discriminate neighboring spots. This makes it particularly difficult to co-localize spots through multiple rounds of hybridization and imaging. Other benefits of a diffraction-limited spot size are that it is suitable for accurate super-resolution structural analysis by, for example, STORM33, DNA-PAINT34-38 or STED39, and also that many image analysis tools assume diffraction-limited spots. ClampFISH 2.0's combination of high amplification, rapid and flexible multiplexing, small spot sizes and low cost enables very high-throughput and quantitative RNA detection. In potential further extensions of the method, clampFISH 2.0 could serve as a platform for higher-throughput sequential labeling schemes and super-resolution imaging.

### Enumerated Embodiments

The following enumerated embodiments are provided, the numbering of which is not to be construed as designating levels of importance.

Embodiment 1 provides a primary click-amplifying FISH (clampFISH) probe comprising:
a first oligonucleotide having
(a) a target-specific oligonucleotide,
wherein the target-specific oligonucleotide is about 30 nucleotides in length and comprises a continuous target-specific binding region;
(b) a first flanking oligonucleotide,
wherein the first flanking oligonucleotide is about 10 nucleotides in length,
wherein the first flanking oligonucleotide is at the 5′ end of the target-specific oligonucleotide;
(c) a second flanking oligonucleotide at the 3′ end of the target-specific sequence,
wherein the second flanking oligonucleotide is about 10 nucleotides in length,
wherein the second flanking oligonucleotide is at the 3′ end of the target-specific sequence; and
wherein the 3′ end of the first oligonucleotide comprises an azide moiety;
a second oligonucleotide having
(d) an amplifier-specific oligonucleotide,
wherein the amplifier-specific oligonucleotide is about 30 nucleotides in length,
(e) a first universal oligonucleotide,
wherein the first universal oligonucleotide is about 18 nucleotides in length,
wherein the first universal oligonucleotide is at the 5′ end of the amplifier-specific oligonucleotide,
(f) a second universal oligonucleotide,
wherein the second universal oligonucleotide is about 10 nucleotides in length,
wherein the second universal oligonucleotide is at the 3′ end of the amplifier-specific oligonucleotide; and
wherein the 5′ end of the second oligonucleotide comprises an alkyne moiety;
wherein the 5′ end of the first oligonucleotide is ligated to the 3′ end of the second oligonucleotide, and.
wherein the 3′ end of the first oligonucleotide can be covalently locked to the 5′ end of the second oligonucleotide using click chemistry to circularize the primary clampFISH probe.

Embodiment 2 provides the primary clampFISH probe of embodiment 1, wherein the first universal oligonucleotide is AGACATTCTCGTCAAGAT (SEQ ID NO: 550).

Embodiment 3 provides the primary clampFISH probe of embodiments 1-2, wherein the second universal oligonucleotide is CTGAGTGTTG (SEQ ID NO: 551).

Embodiment 4 provides the primary clampFISH probe of embodiments 1-3, wherein the azide moiety is N6-(6-Azido) hexyl-dATP.

Embodiment 5 provides the primary clampFISH probe of embodiments 1-4, wherein the azide moiety is added to the 3′ end of the primary clampFISH probe using terminal transferase enzyme.

Embodiment 6 provides the primary clampFISH probe of embodiments 1-5, wherein the alkyne moiety is hexynyl.

Embodiment 7 provides the primary clampFISH probe of embodiments 1-6, wherein the probe is one selected from SEQ ID NO: 453 to SEQ ID NO: 467.

Embodiment 8 provides an amplifier probe comprising:
(a) a backbone comprising about 60 nucleotides,
wherein the backbone is formed by concatenating two oligonucleotides (landing pad 1 and landing pad 2), wherein the landing pad 1 and the landing pad 2 each is about 30 nucleotides in length and comprises a sequence for binding to another amplifier probe;
(b) a first binding arm at the 3′ end of the landing pad 1, wherein the first binding arm is about 15 nucleotides in length;
(c) a second binding arm at the 5′ end of the landing pad 2, wherein the second binding arm is about 15 nucleotides in length;
wherein when the amplifier probe is a secondary amplifier probe, the first and the second binding arm together comprise a sequence that is reverse complementary to the landing pad 1 and/or the landing pad 2 of a tertiary amplifier probe or to an amplifier-specific oligonucleotide of a primary clampFISH probe;
wherein when the amplifier probe is the tertiary amplifier probe, the first and the second binding arm together comprise a sequence that is reverse complementary to the landing pad 1 and/or the landing pad 2 of the secondary amplifier probe,
wherein the 5′ end of the amplifier probe comprises as alkyne moiety and the 3′ end of the amplifier probe comprises an azide moiety,
wherein the 5′ end of the amplifier probe can be covalently locked to its 3′ end to circularize the amplifier probe.

Embodiment 9 provides the amplifier probe of embodiment 8, wherein the GC content of each of the binding arms is about 45% to about 55%.

Embodiment 10 provides the amplifier probe of embodiments 8-9, wherein the azide moiety is N6-(6-Azido) hexyl-dATP.

Embodiment 11 provides the amplifier probe of embodiments 8-10, wherein the alkyne moiety is hexynyl.

Embodiment 12 provides the amplifier probe of embodiments 8-10, wherein the probe is one selected from the SEQ ID NO: 423 to SEQ ID NO: 452.

Embodiment 13 provides a method of exponentially amplifying the signal of a primary click-amplifying FISH (clampFISH) probe, the method comprising:
(a) hybridizing the primary clampFISH probe of embodiments 1-7 to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescence microscopy and/or flow cytometry.

Embodiment 14 provides a method of detecting a target nucleic acid in a sample, the method comprising:
(a) hybridizing the primary clampFISH probe of embodiments 1-7 to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescent microscopy and/or flow cytometry.

Embodiment 15 provides the method of embodiments 13-14, wherein step (f) is repeated 1, 2, 3, 4, 5, 6, 7, 8, 9 or 10 times.

Embodiment 16 provides the method of embodiments 13-15, wherein the length of the primary clampFISH probe is about 109 nucleotides.

Embodiment 17 provides the method of embodiments 13-16, wherein the length of each of the secondary and the tertiary amplifier probes is about 90 nucleotides.

Embodiment 18 provides the method of embodiments 13-17, wherein each of the secondary and the tertiary amplifier probes comprise:
(a) a backbone comprising about 60 nucleotides,
wherein the backbone is formed by concatenating two oligonucleotides (landing pad 1 and landing pad 2), wherein the landing pad 1 and the landing pad 2 each is about 30 nucleotides in length and comprises a sequence for binding to another amplifier probe;
(b) a first binding arm at the 3′ end of the landing pad 1, wherein the first binding arm is about 15 nucleotides in length;
(c) a second binding arm at the 5′ end of the landing pad 2, wherein the second binding arm is about 15 nucleotides in length;
wherein when the amplifier probe is the secondary amplifier probe then the first and the second binding arm together comprise a sequence that is reverse complementary to the landing pad 1 and/or the landing pad 2 of the tertiary amplifier probe or to the amplifier-specific oligonucleotide of the primary clampFISH probe,
wherein when the amplifier probe is the tertiary amplifier probe then the first and the second binding arm together comprise a sequence that is reverse complementary to the landing pad 1 and/or the landing pad 2 of the secondary amplifier probe,
wherein the 5′ end of the amplifier probe comprises as alkyne moiety and the 3′ end of the amplifier probe comprises an azide moiety.

Embodiment 19 provides the method of embodiments 13-18, wherein the set of secondary and tertiary amplifier probes comprises at least 2 probes.

Embodiment 20 provides the method of embodiments 13-19, wherein the length of the readout probe is about 12 to about 20 nucleotides.

Embodiment 21 provides the method of embodiments 13-20, wherein the readout probe can be removed from the amplifier probe.

Embodiment 22 provides the method of embodiments 13-21, wherein the click chemistry agent catalyzes an azide-alkyne cycloaddition thereby circularizing the primary clampFISH probe and covalently locking the secondary and the tertiary amplifier probes around their respective nucleic acid target.

Embodiment 23 provides the method of embodiments 13-22, wherein the click chemistry is catalyzed by copper (I), copper (II) or ruthenium.

Embodiment 24 provides the method of embodiments 13-23, wherein the primary clampFISH probe, the secondary amplifier probes and the tertiary amplifier probes are DNA probes.

Embodiment 25 provides the method of embodiments 13-24, wherein the primary clampFISH probe, the secondary amplifier probes and the tertiary amplifier probes are one selected from the group consisting of peptide nucleic acid (PNA), locked nucleic acid (LNA), and 2′-O-Methyl RNA.

Embodiment 26 provides the method of embodiments 13-25, wherein the target nucleic acid is a DNA or an RNA.

Embodiment 27 provides the method of embodiments 13-26, wherein the RNA is selected from the group consisting of messenger RNA, intronic RNA, exonic RNA, and non-coding RNA.

Embodiment 28 provides the method of embodiments 13-27, wherein the tertiary amplifier probe is identical to the secondary amplifier probe.

Embodiment 29 provides the method of embodiments 13-27, wherein the tertiary amplifier probe is not identical to the secondary amplifier probe.

Embodiment 30 provides the method of embodiments 13-29, wherein the method allows simultaneous detection of multiple target nucleic acids in the sample.

Embodiment 31 provides the method of embodiments 13-30, wherein the method allows detection of the target nucleic acid using a low magnification microscopy.

Embodiment 32 provides the method of embodiments 13-31, wherein the primary clampFISH probe is one selected from SEQ ID NO: 453 to SEQ ID NO: 476.

Embodiment 33 provides the method of embodiments 13-32, wherein the secondary amplifier probe is one selected from SEQ ID NO: 423 to SEQ ID NO: 437.

Embodiment 34 provides the method of embodiments 13-33, wherein the tertiary amplifier probe is one selected from SEQ ID NO: 438 to SEQ ID NO: 452.

Embodiment 35 provides the method of embodiments 13-34, wherein the readout probe is one selected from SEQ ID NO: 358 to SEQ ID NO: 392

Embodiment 36 provides a kit comprising at set of primary click-amplifying FISH (clampFISH) probes of embodiments 1-7, a set of secondary amplifier probes, a set of tertiary amplifier probes, a set of amplifier-specific oligonucleotides, a set of dye-coupled DNA readout probes, a ligase, a hybridization solution, and a click chemistry agent for signal amplification and detection of nucleic acids in a sample and instructions for use thereof.

Embodiment 37 provides a method of synthesizing a primary clampFISH probe by ligating a first oligonucleotide to a second oligonucleotide, wherein
the first oligonucleotide comprises:
(a) a target-specific oligonucleotide,
wherein the target-specific oligonucleotide is about 30 nucleotides in length and comprises a contiguous target-specific binding region;
(b) a first flanking oligonucleotide at the 5′ end of the target-specific oligonucleotide,
wherein the first flanking oligonucleotide comprises about 10 nucleotides;
(c) a second flanking oligonucleotide at the 3′ end of the target-specific sequence,
wherein the second flanking oligonucleotide comprises about 10 nucleotides; and
wherein the 3′ end of the first oligonucleotide comprises an azide moiety;
the second oligonucleotide comprises:
(d) an amplifier-specific oligonucleotide,
wherein the amplifier-specific oligonucleotide is about 30 nucleotides in length,
(e) a first universal oligonucleotide,
wherein the first universal oligonucleotide is about 18 nucleotides in length, and
wherein the first universal oligonucleotide is at the 5′ end of the amplifier-specific oligonucleotide,
(f) a second universal oligonucleotide,
wherein the second universal oligonucleotide is about 10 nucleotides in length, and
wherein the second universal oligonucleotide is at the 3′ end of the amplifier-specific oligonucleotide; and
wherein the 5′ end of the second oligonucleotide comprises an alkyne moiety;
wherein the 5′ end of the first oligonucleotide is ligated to the 3′ end of the second oligonucleotide, and.
wherein the 3′ end of the first oligonucleotide can be covalently locked to the 5′ end of the second oligonucleotide using click chemistry to circularize the primary clampFISH probe.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A primary click-amplifying FISH (clampFISH) probe comprising:
a first oligonucleotide having
(a) a target-specific oligonucleotide,
wherein the target-specific oligonucleotide is about 30 nucleotides in length and comprises a continuous target-specific binding region;
(b) a first flanking oligonucleotide,
wherein the first flanking oligonucleotide is about 10 nucleotides in length,
wherein the first flanking oligonucleotide is at the 5′ end of the target-specific oligonucleotide;
(c) a second flanking oligonucleotide at the 3′ end of the target-specific sequence,
wherein the second flanking oligonucleotide is about 10 nucleotides in length,
wherein the second flanking oligonucleotide is at the 3′ end of the target-specific sequence; and
wherein the 3′ end of the first oligonucleotide comprises an azide moiety;
a second oligonucleotide having:
(d) an amplifier-specific oligonucleotide,
wherein the amplifier-specific oligonucleotide is about 30 nucleotides in length,
(e) a first universal oligonucleotide,
wherein the first universal oligonucleotide is about 18 nucleotides in length,
wherein the first universal oligonucleotide is at the 5′ end of the amplifier-specific oligonucleotide,
(f) a second universal oligonucleotide,
wherein the second universal oligonucleotide is about 10 nucleotides in length,
wherein the second universal oligonucleotide is at the 3′ end of the amplifier-specific oligonucleotide; and
wherein the 5′ end of the second oligonucleotide comprises an alkyne moiety;
wherein the 5′ end of the first oligonucleotide is ligated to the 3′ end of the second oligonucleotide, and.
wherein the 3′ end of the first oligonucleotide can be covalently locked to the 5′ end of the second oligonucleotide using click chemistry to circularize the primary clampFISH probe.

2. The primary clampFISH probe of claim 1, wherein the first universal oligonucleotide is AGACATTCTCGTCAAGAT (SEQ ID NO:550).

3. The primary clampFISH probe of claim 1, wherein the second universal oligonucleotide is CTGAGTGTTG (SEQ ID NO:551).

4. The primary clampFISH probe of claim 1, wherein the azide moiety is N6-(6-Azido) hexyl-dATP.

5. The primary clampFISH probe of claim 4, wherein the azide moiety is added to the 3′ end of the primary clampFISH probe using terminal transferase enzyme.

6. The primary clampFISH probe of claim 1, wherein the alkyne moiety is hexynyl.

7. The primary clampFISH probe of claim 1, wherein the probe is one selected from SEQ ID NO:453 to SEQ ID NO:467.

8. An amplifier probe comprising:
(a) a backbone comprising about 60 nucleotides,
wherein the backbone is formed by concatenating two oligonucleotides (landing pad 1 and landing pad 2), wherein the landing pad 1 and the landing pad 2 each is about 30 nucleotides in length and comprises a sequence for binding to another amplifier probe;
(b) a first binding arm at the 3′ end of the landing pad 1, wherein the first binding arm is about 15 nucleotides in length;
(c) a second binding arm at the 5′ end of the landing pad 2, wherein the second binding arm is about 15 nucleotides in length;
wherein when the amplifier probe is a secondary amplifier probe, the first and the second binding arm together comprise a sequence that is reverse complementary to the landing pad 1 and/or the landing pad 2 of a tertiary amplifier probe or to an amplifier-specific oligonucleotide of a primary clampFISH probe;
wherein when the amplifier probe is the tertiary amplifier probe, the first and the second binding arm together comprise a sequence that is reverse complementary to the landing pad 1 and/or the landing pad 2 of the secondary amplifier probe,
wherein the 5′ end of the amplifier probe comprises as alkyne moiety and the 3′ end of the amplifier probe comprises an azide moiety,
wherein the 5′ end of the amplifier probe can be covalently locked to its 3′ end to circularize the amplifier probe.

9. The amplifier probe of claim 8, wherein the GC content of each of the binding arms is about 45% to about 55%.

10. The amplifier probe of claim 8, wherein the azide moiety is N6-(6-Azido) hexyl-dATP.

11. The amplifier probe of claim 8, wherein the alkyne moiety is hexynyl.

12. The amplifier probe of claim 8, wherein the probe is one selected from the SEQ ID NO: 423 to SEQ ID NO: 452.

13. A method of exponentially amplifying the signal of a primary click-amplifying FISH (clampFISH) probe, the method comprising:
(a) hybridizing the primary clampFISH probe of claim 1 to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescence microscopy and/or flow cytometry.

14. A method of detecting a target nucleic acid in a sample, the method comprising:
(a) hybridizing the primary clampFISH probe of claim 1 to a target nucleic acid in a sample,
(b) contacting the primary clampFISH probe with a secondary amplifier probe;
(c) adding a click chemistry agent that circularizes the primary clampFISH probe and covalently locks the secondary amplifier probe to the amplifier-specific oligonucleotide of the primary clampFISH probe to form a secondary sample;
(d) contacting the secondary sample with a set of tertiary amplifier probes that bind to each secondary amplifier probe and adding a click chemistry agent that covalently locks the set of tertiary amplifier probes to each secondary amplifier probe to form a tertiary sample;
(e) contacting the tertiary sample with a set of secondary amplifier probes that bind to each tertiary amplifier probe and adding a click chemistry agent that covalently locks the secondary amplifier probes to each tertiary amplifier probe; and,
(f) repeating steps (d) and (e) until a desired amplified scaffold is achieved;
(g) hybridizing a fluorescent dye-coupled DNA readout probe to the secondary and/or tertiary amplifier probes of the scaffold, wherein the signal from the readout probes is detected by a fluorescent microscopy and/or flow cytometry.

15. The method of claim 13, wherein step (f) is repeated 1, 2, 3, 4, 5, 6, 7, 8, 9 or 10 times.

16. The method of claim 13, wherein the length of the primary clampFISH probe is about 109 nucleotides.

17. The method of claim 13, wherein the length of each of the secondary and the tertiary amplifier probes is about 90 nucleotides.

18. The method of claim 17, wherein each of the secondary and the tertiary amplifier probes comprise:
(a) a backbone comprising about 60 nucleotides,
wherein the backbone is formed by concatenating two oligonucleotides (landing pad 1 and landing pad 2), wherein the landing pad 1 and the landing pad 2 each is about 30 nucleotides in length and comprises a sequence for binding to another amplifier probe;
(b) a first binding arm at the 3′ end of the landing pad 1, wherein the first binding arm is about 15 nucleotides in length;
(c) a second binding arm at the 5′ end of the landing pad 2, wherein the second binding arm is about 15 nucleotides in length;
wherein when the amplifier probe is the secondary amplifier probe, the first and the second binding arm together comprise a sequence that is reverse complementary to the landing pad 1 and/or the landing pad 2 of the tertiary amplifier probe or to the amplifier-specific oligonucleotide of the primary clampFISH probe,
wherein when the amplifier probe is the tertiary amplifier probe, the first and the second binding arm together comprise a sequence that is reverse complementary to the landing pad 1 and/or the landing pad 2 of the secondary amplifier probe,
wherein the 5′ end of the amplifier probe comprises as alkyne moiety and the 3′ end of the amplifier probe comprises an azide moiety.

19. The method of claim 13, wherein the set of secondary and tertiary amplifier probes comprises at least 2 probes.

20. The method of claim 13, wherein the length of the readout probe is about 12 to about 20 nucleotides.

21. The method of claim 13, wherein the readout probe can be removed from the amplifier probe.

22. The method of claim 13, wherein the click chemistry agent catalyzes an azide-alkyne cycloaddition thereby circularizing the primary clampFISH probe and covalently locking the secondary and the tertiary amplifier probes around their respective nucleic acid target.

23. The method of claim 13, wherein the click chemistry is catalyzed by copper (I), copper (II) or ruthenium.

24. The method of claim 13, wherein the primary clampFISH probe, the secondary amplifier probes and the tertiary amplifier probes are DNA probes.

25. The method of claim 13, wherein the primary clampFISH probe, the secondary amplifier probes and the tertiary amplifier probes are one selected from the group consisting of peptide nucleic acid (PNA), locked nucleic acid (LNA), and 2′-O-Methyl RNA.

26. The method of claim 13, wherein the target nucleic acid is a DNA or an RNA.

27. The method of claim 26, wherein the RNA is selected from the group consisting of messenger RNA, intronic RNA, exonic RNA, and non-coding RNA.

28. The method of claim 13, wherein the tertiary amplifier probe is identical to the secondary amplifier probe.

29. The method of claim 13, wherein the tertiary amplifier probe is not identical to the secondary amplifier probe.

30. The method of claim 13, wherein the method allows simultaneous detection of multiple target nucleic acids in the sample.

31. The method of claim 13, wherein the method allows detection of the target nucleic acid using a low magnification microscopy.

32. The method of claim 13, wherein the primary clampFISH probe is one selected from SEQ ID NO:453 to SEQ ID NO:467.

33. The method of claim 13, wherein the secondary amplifier probe is one selected from SEQ ID NO:423 to SEQ ID NO:437.

34. The method of claim 13, wherein the tertiary amplifier probe is one selected from SEQ ID NO:438 to SEQ ID NO:452.

35. The method of claim 13, wherein the readout probe is one selected from SEQ ID NO:358 to SEQ ID NO:392.

36. A kit comprising a set of primary probes comprising the primary click-amplifying FISH (clampFISH) probe of claim 1, a set of secondary amplifier probes, a set of tertiary amplifier probes, a set of amplifier-specific oligonucleotides, a set of dye-coupled DNA readout probes, a ligase, a hybridization solution, and a click chemistry agent for signal amplification and detection of nucleic acids in a sample and instructions for use thereof.

37. A method of synthesizing a primary clampFISH probe by ligating a first oligonucleotide to a second oligonucleotide, wherein
the first oligonucleotide comprises:
(a) a target-specific oligonucleotide,
wherein the target-specific oligonucleotide is about 30 nucleotides in length and comprises a contiguous target-specific binding region;
(b) a first flanking oligonucleotide at the 5′ end of the target-specific oligonucleotide, wherein the first flanking oligonucleotide comprises about 10 nucleotides;
(c) a second flanking oligonucleotide at the 3′ end of the target-specific sequence, wherein the second flanking oligonucleotide comprises about 10 nucleotides; and
wherein the 3′ end of the first oligonucleotide comprises an azide moiety;
wherein the second oligonucleotide comprises:
(d) an amplifier-specific oligonucleotide,
wherein the amplifier-specific oligonucleotide is about 30 nucleotides in length,
(e) a first universal oligonucleotide,
wherein the first universal oligonucleotide is about 18 nucleotides in length, and
wherein the first universal oligonucleotide is at the 5′ end of the amplifier-specific oligonucleotide,
(f) a second universal oligonucleotide,
wherein the second universal oligonucleotide is about 10 nucleotides in length, and
wherein the second universal oligonucleotide is at the 3′ end of the amplifier-specific oligonucleotide; and
wherein the 5′ end of the second oligonucleotide comprises an alkyne moiety;
wherein the 5′ end of the first oligonucleotide is ligated to the 3′ end of the second oligonucleotide, and,
wherein the 3′ end of the first oligonucleotide can be covalently locked to the 5′ end of the second oligonucleotide using click chemistry to circularize the primary clampFISH probe.
